(19) United States
(12) Patent Application Publication
(10) Pub. No.: US 2024/0188876 A1
(43) Pub. Date: Jun. 13, 2024

(54) SYSTEM AND METHOD FOR DETECTION OF CARDIAC ARRHYTHMIA USING ENCODING ECG SIGNALS

(71) Applicant: The Research Foundation for The State University of New York, Binghamton, NY (US)

(72) Inventor: Kanad Ghose, Vestal, NY (US)

(21) Appl. No.: 18/539,233

(22) Filed: Dec. 13, 2023

Related U.S. Application Data
(60) Provisional application No. 63/432,089, filed on Dec. 13, 2022.

(51) Int. Cl. A61B 5/364; A61B 5/00; A61B 5/339; A61B 5/352; A61B 5/353; A61B 5/355; A61B 5/366

(52) U.S. Cl. CPC A61B 5/364; A61B 5/0006; A61B 5/002; A61B 5/339; A61B 5/352; A61B 5/353; A61B 5/355; A61B 5/366; A61B 2560/0209

(57) ABSTRACT

An electrocardiogram sensing system, comprising: an input port configured to receive an electrocardiogramal; at least one automated processor, configured to: process a representation of the electrocardiographic signal to determine an electrocardiographic waveform for a single heartbeat; and encode a set of quantitative parameters from the electrocardiographic waveform, dependent on geometric relationships, e.g., amplitude, width and relative spacing of components of the electrocardiographic waveform; and a wireless communication device, configured to communicate the encoded set of quantitative parameters.

### CROSS REFERENCE TO RELATED APPLICATIONS

The present application is a non-provisional of, and claims benefit of priority under 35 U.S.C. § 119(c) from U.S. Patent Application No. 63/432,089, filed Dec. 13, 2023, the entirety of which is expressly incorporated herein by reference.

### FIELD OF THE INVENTION

The present invention relates to the field of distributed electrocardiographic signal encoding and analysis, and arrythmias detection using encoded ECG signals.

### INCORPORATION BY REFERENCE

Citation or identification of any reference herein, or any section of this application shall not be construed as an admission that such reference is available as prior art. The disclosure of each publication and patent listed or referenced herein are hereby incorporated by reference in their entirety in this application, and shall be treated as if the entirety thereof forms a part of this application. Such references are provided for their disclosure of technologies as may be required to enable practice of the present invention, to provide written description for claim language, to make clear applicant's possession of the invention with respect to the various aggregates, combinations, permutations, and subcombinations of the respective disclosures or portions thereof (within a particular reference or across multiple references) in conjunction with the combinations, permutations, and subcombinations of various disclosure provided herein, to demonstrate the technological non-abstract nature of the inventions claimed, and for any other purpose. Except as expressly indicated, the scope of the invention is inclusive, and therefore the disclosure of a technology or teaching within these incorporated materials is intended to encompass that technology or teaching as being an option of, or an addition to, other disclosure of the present invention. Likewise, the combination of incorporated teachings consistent with this disclosure is also encompassed. The citation of references is intended to be part of the disclosure of the invention, and not merely supplementary background information. While cited references may be prior art, the combinations thereof and with the material disclosed herein is not admitted as being prior art.

The incorporation by reference does not extend to teachings which are inconsistent with the invention as expressly described herein as being essential. The incorporated references are rebuttable evidence of a proper interpretation of terms, phrases, and concepts employed herein by persons of ordinary skill in the art. No admission is made that any incorporated reference is analogous art to the issues presented to the inventor, and the selection, combination, and disclosure of these disparate teachings is itself a part of the invention herein disclosed.

### BACKGROUND OF THE INVENTION

Recent years have seen the emergence of wearable ECG sensors with a small factor, which can acquire ECG signals and transmitting the acquired signal wirelessly to a host device in real-time. An example of such a device is called SEP (Smart ECG Patch), which also implements a variety of power savings technique to prolong the device's operation time on the battery (Mittal et al., 2020).

Arrhythmia is an abnormal heart rhythm or heart rate (HR). Clinicians identify arrhythmia by observing the irregular changes in HR and morphological changes in ECG signals. Arrhythmias, such as the left bundle branch block, right bundle branch block, premature ventricular contraction, and atrial premature beat exhibit significant changes in the characteristics of the QRS complex. Other arrhythmias like paced beat and many others are identified primarily by observing irregularities in instantaneous HR.

Arrhythmias are often categorized into four groups: extra beats, supraventricular tachycardias, ventricular arrhythmias and bradyarrhythmias Extra beats include premature atrial contractions, premature ventricular contractions and premature junctional contractions. Supraventricular tachycardias include atrial fibrillation, atrial flutter and paroxysmal supraventricular tachycardia. Ventricular arrhythmias include ventricular fibrillation and ventricular tachycardia. Bradyarrhythmias are due to sinus node dysfunction or atrioventricular conduction disturbances. Arrhythmias are due to problems with the electrical conduction system of the heart.

In general, heart arrhythmias are grouped by the speed of the heart rate. For example: Tachycardia is a fast heart (the heart rate is greater than 100 beats a minute); and bradycardia is a slow heartbeat (the heart rate is less than 60 beats a minute).

Types of tachycardias include:
Atrial fibrillation (A-fib). Chaotic heart signaling causes a rapid, uncoordinated heart rate. The condition may be temporary, but some A-fib episodes may not stop unless treated. A-fib is associated with serious complications such as stroke.
Atrial flutter. Atrial flutter is similar to A-fib, but heartbeats are more organized. Atrial flutter is also linked to stroke.
Supraventricular tachycardia. Supraventricular tachycardia is a broad term that includes arrhythmias that start above the lower heart chambers (ventricles). Supraventricular tachycardia causes episodes of a pounding heartbeat (palpitations) that begin and end abruptly.
Ventricular fibrillation. This type of arrhythmia occurs when rapid, chaotic electrical signals cause the lower heart chambers (ventricles) to quiver instead of contacting in a coordinated way that pumps blood to the rest of the body. This serious problem can lead to death if a normal heart rhythm isn't restored within minutes. Most people who have ventricular fibrillation have an underlying heart disease or have experienced serious trauma.
Ventricular tachycardia. This rapid, regular heart rate starts with faulty electrical signals in the lower heart chambers (ventricles). The rapid heart rate doesn't allow the ventricles to properly fill with blood. As a result, the heart can't pump enough blood to the body. Ventricular tachycardia may not cause serious problems in people with an otherwise healthy heart. In those with heart disease, ventricular tachycardia can be a medical emergency that requires immediate medical treatment.

### Types of Bradycardias Include

Sick sinus syndrome. The sinus node is responsible for setting the pace of the heart. If it doesn't work properly, the heart rate may alternate between too slow (bradycardia) and too fast (tachycardia). Sick sinus syndrome can be caused by scarring near the sinus node that's slowing, disrupting or blocking the travel of impulses. Sick sinus syndrome is most common among older adults.

Conduction block. A block of the heart's electrical pathways can cause the signals that trigger the heartbeats to slow down or stop. Some blocks may cause no signs or symptoms, and others may cause skipped beats or bradycardia.

Premature heartbeats are extra beats that occur one at a time, sometimes in patterns that alternate with the normal heartbeat. The extra beats may come from the top chamber of the heart (premature atrial contractions) or the bottom chamber (premature ventricular contractions). Premature heartbeats may occur when resting. Sometimes premature heartbeats are caused by stress, strenuous exercise or stimulants, such as caffeine or nicotine.

Arrhythmia may also be classified by mechanism (automaticity, re-entry, triggered) or duration (isolated premature beats; couplets; runs, that is 3 or more beats; non-sustained=less than 30 seconds or sustained=over 30 seconds). Arrhythmias may also be classified by site of origin:

Atrial arrhythmia: Sinus bradycardia, Sinus arrhythmia, Sinus tachycardia, Premature atrial contractions (PACs), Wandering atrial pacemaker, Atrial tachycardia, Multifocal atrial tachycardia, Supraventricular tachycardia (SVT), Atrial flutter, Atrial fibrillation (Afib), AV nodal reentrant tachycardia.

Junctional arrhythmia: AV nodal reentrant tachycardia, Junctional rhythm, Junctional tachycardia. Premature junctional contraction.

Ventricular arrhythmia: Premature ventricular contractions (PVCs), sometimes called ventricular extra beats (VEBs). Premature ventricular beats occurring after every normal beat are termed ventricular bigeminy PVCs that occur at intervals of 2 normal beats to 1 PVC, or 1 normal beat to 2 PVCs, are termed “PVCs in trigeminy”. Groups of three premature ventricular beats are called triplets and are considered a brief run of non-sustained ventricular tachycardia (NSVT); if the grouping lasts for more than 30 seconds, it is considered sustained ventricular tachycardia (VT), Accelerated idioventricular rhythm, Monomorphic ventricular tachycardia, Polymorphic ventricular tachycardia, Ventricular fibrillation, Torsades de pointes, Arrhythmogenic right ventricular dysplasia, Re-entry ventricular arrhythmia.

Heart blocks: These are also known as AV blocks, because the vast majority of them arise from pathology at the atrioventricular node. They are the most common causes of bradycardia: First-degree heart block, which manifests as PR prolongation, Second-degree heart block, Type 1 Second degree heart block, also known as Mobitz I or Wenckebach, Type 2 Second degree heart block, also known as Mobitz II, Third-degree heart block, also known as complete heart block, First, second, and third-degree blocks also can occur at the level of the sinoatrial junction. This is referred to as sinoatrial block typically manifesting with various degrees and patterns of sinus bradycardia.

Real time arrhythmia detection can be beneficial in situations such as post cardiac catheterization wherein monitoring the possibility of real time arrhythmia can be critical. Agyeman et al. 2022, Alfaras, et al. 2019, Chang et al. 2018, Clark et al, 2018, Faust et al. 2020, Gotlibovych et al. 2018, Gupta et al 2020a, Gupta et al, 2020b, Hirsch et al. 2021, Hui et al., 2021, Ingolfsson et al, 2021, Jin et al., 2020, Kim et al, 2022, Liu et al, 2022, Marsili et al, 2020, Meng et al., 2021, Pereira et al. 2020, Petmezas et al. 2021, Ramesh et al. 2021, Shao et al. 2020, Vizitiu et al. 2021, Xiong et al. 2022, Yao et al. 2020, Yao et al. 2018, Yilirim et al. 2018.

Individuals under the influence of alcohol, tobacco and recreational drugs can be at a potential risk of developing arrhythmia. Individuals who take prescribed medication, such as Abiraterone Acetate, Enflurane, Iopromide, are at risk of arrhythmias as well. Other factors that lead to arrhythmia and where real time arrhythmia detection will be useful are discussed in (Mayo, 2020).

Arrhythmia detection and classification has been the subject of significant studies, most of which tend to use the entire ECG waveform for the classification. Some feature extraction techniques presented in (Tantawi et al. 2015, Zadeh et al. 2010, Kim et al. 2009, Kallas et al. 2012, Wang et al. 2013, Sufi et al., 2010, and Mukhopadhyay et al. 2012) prove to be not amenable to real time operation because the length and number of features extracted are large, and may require extended duration sampling and analysis. A larger number of features can lead to commensurate power-consuming data transmission from a wearable ECG device or require the transmission of the complete ECG waveforms, shortening the operation time of the wearable sensor between battery replacements or charging, are defeating the ability to package it in a small convenient form factor, e.g., wearable. This is because the energy consumption of a wearable ECG device is dominated by the energy expended in wireless data transmission. Arrhythmia detection techniques presented in (Huanhuan et al. 2014, Kiranyaz et al. 2015a, Kiranyaz et al. 2015b, Labati et al. 2019, Luz et al. 2016, Zadeh et al. 2010) perform a binary classification of arrhythmia by only looking the heart rate variations and are thus incapable of identifying specific types/classes of arrhythmias. These techniques use convolutional neural network models that use the full ECG signals as input, which requires the wearable sensor to transmit all samples of the full ECG signal, leading to heavy battery drain. Other arrhythmia detection techniques use feature extraction methods to derive dominant ECG features for arrhythmia classification (Zadeh et al, 2010, Kallas et al. 2012, Wang et al. 2013, Sufi et al, 2010, Yeh et al. 2012). However, the features extracted in these techniques are significant in size and, in some cases, require complex computations on the sensor for extraction. Both factors contribute to added battery drain and an increase in computational latency.

The heart has a special electrical system called the cardiac conduction system. It controls the rhythm and rate of the heartbeat. A heartbeat is made by the sequenced contraction of atria and ventricles. A heartbeat is also referred to as a cardiac cycle. With each heartbeat, an electrical signal travels from the top of the heart to the bottom. The contraction of the heart is triggered by the distribution of the electrical signal along conduction pathways, which then selectively excite contractile tissue. Each normal cardiac cycle is initiated by the electrical impulses generated by the sinoatrial (SA) node located in the right atrium of the heart. The ECG registers a positive deflection in amplitude due to depolarization. The next step after initiation at SA node, the electrical impulse reaches atrioventricular (AV) node. In a normal cardiac cycle, it takes approximately 50 milliseconds to cover the distance from SA node to AV node. At the AV node, there is a critical pause of approximately 100 milliseconds. This pause is critical as it allows for other cardiac muscles to contract to pump blood into the ventricles. Originating from the AV node, the electrical impulses travel divided into the left and right bundle branches. The passage of electrical impulse through these branches takes a total of approximately 25 milliseconds to reach the apex of the heart. The apex of the heart has fast conductive fibers called the Purkinje Fibers. These are spread across the ventricles causing the ventricular contraction. It takes about 75 milliseconds for the electrical impulse to reach the ventricular muscles from these fibers. Finally, the electrical impulse causes the ventricular contraction (also known as ventricular depolarization) which pumps the blood out of the heart and completes one full cardiac cycle. The total time elapsed from the initiation of electrical impulse at the SA node until ventricular depolarization is approximately 225 milliseconds for a normal cardiac cycle.

An ECG signal can be plotted in two-dimensions, where the y-axis represents the signal amplitude and x-axis represents time. A full ECG waveform cycle, or pulse is shown in FIG. 2. The ECG waveform is composed of five individual positive and negative deflections along the x-axis. Each individual waveform represents conduction of electrical impulses through different parts of the heart. From FIG. 2, the P-wave is a shown as a positive deflection representing the SA initiation of the electrical impulse. The QRS complex shows the ventricular electrical conductivity of the heart, caused by ventricular depolarization (contraction), and the T-wave is seen due to ventricular repolarization (relaxation). The R-wave, or R-peak has the highest amplitude in an ECG waveform. SEP focuses on detecting the QRS complex of the ECG. Detection of the amplitude of the QRS complex and its temporal position may be performed in real time by a microcontroller. The R-peaks are detected as described in (Mittal et al., 2020). The Q- and S- waves are detected using the Pan Tompkins algorithm (Pan et al. 1985).

Recent advances in wearable technology have enabled the realization of a variety of smart sensors for health monitoring aimed at individual users. Extensive studies (Baig et al. 2013, Islam et al. 2015, Soh et al. 2015, Lobodzinski et al. 2012) have discussed the challenges in wearable system for healthcare applications. These studies show that long battery life is imperative for the end-user and a comfortable wearable solution is necessary. Furthermore, it desirable to have a wearable sensor that can be easily worn under apparel. Such sensors can collect important data like ECG, respiratory rate and calculate human performance/clinical parameters such as Heart Rate (HR), Heart Rate Variability (HRV), and more. The acquired data can be used for performance and wellness monitoring, including the reporting of clinically meaningful variations of key sensed data for healthy subjects and subjects at risk, elderly subjects and subjects engaged in potentially stressful activities. Some desirable attributes of these wearable sensors are: (a) power consumption needs to be grossly limited, as they are battery powered; (b) the data acquisition process should be both accurate and reliable; (c) the device should be as unobtrusive as possible for comfort and cosmetics, and (d) the privacy of the acquired data must be ensured, as the acquired data is monitored remotely.

In recent years, wearable ECG or heart rate sensors have entered the market in various forms and have also been developed in the research community. Generally, they appear to have a variety of limitations. ECG sensors, such as Biostamp (Sen-Gupta et al. 2019) are incapable of acquiring and sending ECG data continuously—they need to be taken off the subject for transferring the acquired data stored on-board to a host for eventual processing. CALM-M requires external interrogation to transmit data to a host for interpretation. The sensors of (Poliks et al. 2016) and (Shin et al. 2018) can send ECG signals continuously but lack aggressive power management and on-board analytics thereby limiting their usefulness in longer-term monitoring situations. The device described in (Wang et al. 2015) limits transmission power based on received signal strength (as SEP does), but other forms of power management are lacking.

Devices such as Apple watch series 4, Samsung Galaxy Watch 4 and 5, and Amazfit Health band, compared in (Jovin et al. 2019), require the wearer's intervention to transmit ECG signals; a consequence of the limitations of on-board electronics to save power. Other ECG-only-on-demand sensors include AliveCor Kardiaband, Omron Complete and WIWE—all described in (Juvin et al. 2019). Many ECG sensors are either bulky (Jin-ling et al 2013, Krachunov et al. 2017) or require belts or belt-like harnesses carrying the sensors to be worn, such as Qardiocore (Jovin et al. 2019), Eco-ECG (Park et al, 2016), HeartBit (Jovin et al. 2019) and the sensor of (Shin et al. 2018), while others require wrist straps on both hands (Krachunov et al. 2017).

On-board analytics on a wearable ECG sensor is critical for monitoring the subject's condition. Some newer wearable sensors provide this capability for atrial fibrillation only, and otherwise require analysis on an off-board device (such as cell phones, for most of the sensors described earlier) or the Cloud (Akrivopoulos et al. 2017, Coulter et al. 2017). Some cloud-based solutions, such as (Gusev et al. 2017), do not really have long battery life and any aggressive power monitoring implementations. Other devices (Fensli et al. 2005, Anliker et al. 2004, Yang et al. 2008, Spanò et al. 2016, Chowdhury et al. 2018, Baba et al. 2018, Jang et al. 2018) have most, or all, rely on off-board analysis of the acquired ECG signals on a PC or a mobile system. Often the processing is not done as the signal in real-time, making these devices unusable for critical, live monitoring situations. Also, these devices do not have any artifact detection induced by motion, or aggressive noise cancelling techniques.

### SUMMARY OF THE INVENTION

The present technology provides a wearable or otherwise miniature, real time sensor and processor, called Smart ECG Patch (SEP), which encodes an acquired ECG signal onboard the SEP, using a small number of parameters, e.g., only 14 integer parameters, for each ECG cycle, prior to transmission. This represents an improvement for example, over the what was reported in (Mittal et al., 2020), especially in terms of operating time. Because of the encoding, further energy savings are realized through a dramatic reduction in the amount of data to be transmitted to the host.

A deep learning system called Arrhythmia Classification using Encoded ECG Signal (ACES) performs arrhythmia classification in real time using the encoded data, e.g., received on a Bluetooth host device. The SEP device is a 2-electrode, low-power, discretely wearable, real-time ECG sensor patch implemented on a flexible substrate. SEP has a processor that removes motion artifacts and is capable of simple on-board analytics (Mittal et al., 2020).

FIG. 1 shows the exposed SEP prototype, which shows the discreet electrical components and a Bluetooth Low-Energy (BLE) microcontroller, as described in (Mittal et al., 2020). All circuitry and copper connections on the body side are coated with a thick insulating layer and only the two electrodes remain exposed to contact the wearer's body when the patch is worn. SEP acquires and sends the ECG signal and the computed heart rate (HR) and heart rate variability (HRV) values to a host device via a secure Bluetooth link in real time. ACES enables the capabilities and battery lifetime of SEP to be extended further for real time, critical cardiac health monitoring. The energy savings realized with ACES comes from eliminating the need to transmit of all sampled signals for a single ECG waveform cycle and instead transmit only the 14 integer values for the encoded representation of an ECG pulse. Specifically, instead of transmitting 400 integer samples per ECG pulse or ECG cycle for a 400 Hz sampling rate, only 14 integer parameters must be transmitted over the same duration for the encoded representation. This leads to a dramatic reduction in the energy drawn from a battery within the SEP.

ACES focuses on autonomous and continuous arrhythmia classification based on morphological changes in the QRS complex and irregular instantaneous heart rate. ACES system uses BILSTM (Shester et al. 1997) layers and is trained to detect all types of arrhythmias and classifies them into seven separate classes (6 classes for arrhythmia and a class for normal sinus rhythm) using only the 14 encoded features of the ECG pulse. (A normal sinus rhythm is defined as the rhythm of a healthy heart. In adults, normal sinus rhythm is supposed to have a resting HR of 60 beats-per-minute (bpm) to 100 bpm.). ACES removes the computational burden of decoding the encoded ECG signal for arrhythmia classification, thus enabling the detection to run in real time without any significant latency. The MIT-BIH arrhythmia dataset (Goldberger et al. 2000, Moody et al 2001) was used for training the ACES classifier model. As discussed below, the model is evaluated using both archival and real time ECG from human subjects.

ACES can classify arrhythmia using only 14 easy-to-derive features from QRS complex per ECG pulse. By extracting only 14 QRS complex-based parameters from a full ECG pulse, and sending parameters instead of continuous samples of the full ECG waveform, the data transmission to the host is significantly reduced. The data reduction also speeds up the convergence of the training for the ACES classifier.

In SEP, a moving window for Q-peak and S-peak detection is preferably set at 150 milliseconds. This value is chosen to include the QRS width in a normal sinus rhythm which is less than 120 milliseconds (mS), as described in (Kurl et al. 2012). The extra 30 mS allows for the QRS complex to be detected in certain cases of arrhythmias where the QRS complex has prolonged duration (>120 mS) between its Q-peak and S-peak. Every time a single R-peak is detected by SEP, a 150 mS-long window is therefore extracted from the signal to identify the Q- and S-peaks within it. In this window, all the values may be squared to enhance the Q- and S-peaks, followed by the relative amplitude comparison with R-peak to identify the Q-peak and the S-peak. The detected QRS complex is then analyzed further as described below.

The present technology provides a self-contained smart ECG wearable device, e.g., a Smart ECG Patch (SEP) in the form of a flexible patch with embedded electrodes (e.g., gold), and onboard electronics for acquiring ECG signals, digitizing the ECG signal and processing the digitized signal for detecting inevitable noise/motion artifacts and deriving the heart rate and heart rate variability parameters. The patch may communicate the ECG signal and the computed heart rate/heart rate variability values to a host via a secure Bluetooth link. The onboard electronics may also be capable of generating alert signals when the heart rate and heart rate variability cross user-specific pre-specified thresholds; other alarm thresholds can also be set. The patch can be configured remotely to operate in a variety of power savings mode where data is transmitted only under the configured alarm conditions. Further, the SEP can be placed on the lower left ribcage of the subject, displaced from the subject's heart and hidden under normal clothing, a feature that makes it attractive to those who are reluctant to wear similar monitors because the sensors are visible to others. The SEP may be adhered to the subject's body by using a double-sided adhesive tape with cutouts for the electrode areas. The cutout areas may enclose and contain a medical contact gel.

The technology was implemented in a prototype, that uses discrete electronics component in packages, though the design may be highly integrated. The design can be implemented as, for example, a 2 inch square patch, shown in FIG. 1. With the use of bare dies on a flexible substrate, the sensor can be realized as a 1.25 inch by 2-inch (or smaller) patch. As shown in FIG. 1, the microprocessor, memory, and Bluetooth functions may be provided on a modular circuit 2, which is then placed on the flexible substrate 1. Alternately, the microprocessor and/or transceiver may be mounted directly on the substrate, if appropriate electromagnetic interference suppression and especially in the case of the antenna 3, impedance control is achieved. The flexible substrate has two electrodes 4, 5 formed on the rear side, which produce signals processed by an analog front-end circuit 6. An analog filter circuit 7 is provided implemented as additional components provided on the substrate. A coin cell lithium battery 8 powers the system.

The SEP generally differs from ECG sensors developed in the research community that lack onboard R-peak detection capabilities (Shin et al. 2018, [41-49], Coulter et al. 2017, Nemati et al. 2012, Khalaf et al. 2017, Jani et al. 2017, Krachunov et al. 2017, Wang et al. 2016, Wang et al. 2015, Sylvester et al. 2017). An example of such sensors is the passive ECG signal acquisition device on a belt with widely separated electrodes and designed to operate continuously that lacks power management features, and on-board active signal processing for reducing motion artifacts and for estimating HR/HRV (Shin et al. 2018). Another example is described in (TAGecg). Many sensors also rely on off-board processing for deriving the HR and HRV values, and use more than two electrodes Nemati et al. 2012, Khalaf et al. 2017, Jani et al. 2017, Wang et al. 2015, Sylvester et al. 2-17). SEP also differs from existing research prototypes or products that are not designed for continuous operation or require hand pressure, additional support or belts to engage the electrode reliably with the subject, nor requires acquisition to be interrupted to permit the sensor to be relocated for downloading the acquired ECG signal (Shimmer3, Jovin et al. 2019). ECG-Watch (Randazzo et al. 2019) is a wristwatch-like device for acquiring and sending signals to a host device on interrogation for further analytics and incorporates embedded analytics algorithm for detecting atrial fibrillation (AFib).

SEP as implemented in the prototype lacks the onboard AFib detection capability of an ECG watch, but has about one-third of the power requirements of the ECG-Watch for transmission as well as acquisition, and can run for much longer durations, perform other on-board signal analytics, trigger transmissions on its own on sensing abnormal conditions and can be worn discreetly under the clothing. Of course, an atrial fibrillation-detection algorithm may be implemented in SEP, with consequent increase in processing power. Some other remote monitoring ECG systems (Yang et al. 2016, Yang et al. 2008, Spanò et al. 2008) used for telemedicine and home care solutions do not specify aggressive power management techniques and implement most of the signal post processing offboard on a wireless host like a PC.

An earlier version of the sensor that was only capable of ECG signal acquisition was described in (Poliks 2016), where processing was done offboard on a Bluetooth host, and had no incorporation of any power management technique. In contrast, the present technology provides an enhanced system, specifically, onboard software algorithms used to detect R-peaks in QRS complex of an ECG signal, for detecting noise/motion artifacts, and reducing overall power consumption very aggressively.

Smartwatches typically provide continuous real-time photoplethymography (PPG), but only on-demand ECG and consequently intermittent ECG analysis capability. For example, this is because both arms need to be engaged concurrently to obtain a two-lead ECG signal.

On-board signal acquisition with automatic artifact removal and its analysis on-board (heart rate, heart rate variability) permit generation of alarms during monitoring and for conserving power to prolong battery life. This is precisely where SEP appears to have a significant advantage over other sensors described above, in addition to being easily wearable. While it is preferred that the SEP transfer the parameters to a mobile device, such as a smartphone for arrythmia processing, with consequent increase in power consumption for algorithm processing, but reduction in communication burden, the arrythmia processing may be included within the SEP.

A battery-operated wearable device capable of acquiring electrocardiogram (ECG) signals and data for the wearer is therefore provided. The device performs local signal processing to remove noise and signal artifacts, to extract parameters from the ECG, and optionally generate additional clinical data of interest, to generate alarm signals based on pre-specified parameter thresholds, and communicate the parameters and/or clinical data of interest to a remote device. The device may also detect sudden movements by the wearer.

All acquired data, parameters and alarms generated on the device can be sent to a host device using a secure wireless link, e.g., a personal area network or body area network, for example, Bluetooth (en.wikipedia.org/wiki/Bluetooth), BLE (en.wikipedia.org/wiki/Bluetooth_Low_Energy) or Zigbee (en.wikipedia.org/wiki/Zigbee). The device is also capable of operating in a number of modes to conserve battery power and to prolong its operation time without recharging or replacing the battery on the device.

A microcontroller within the patch device controls the acquisition of the ECG signal from the wearer, performs preprocessing to reduce signal noise and motion artifacts to recover a usable ECG signal and control the overall mode of operations of the sensor, including power management, communication with a host device and generation of ECG-extracted parameters and/or alarms.

The device addresses an important need to have a small and easily wearable, wire-free ECG sensor that operates for a long time on a single small non-rechargeable battery (or on a charged small rechargeable battery) and is capable of generating ECG data, parameters of interest and optionally alarms.

The data, once transmitted, may be further processed or displayed on a Bluetooth or BLE host (or other host device), or further relayed to a server, cloud, or destination for processing, analysis, storage/archive, and user interface.

In one implementation, the ECG patch, through a smartphone or other relay device, may relay ECG signals of interest to a processing center, a physician, and/or an emergency medical services provider. The destination may be preprogrammed, determined within the ECG patch, determined by the host device, or determined by a remote server.

The device is preferably self-contained, as a wire-free device and in its inclusion of local signal processing for noise and artifact removal, ECG parameter extraction/encoding, for e.g., detecting sudden movements of the wearer that indicate potential emergencies like a fall or collapse of the wearer, and optionally generating alarms based on sensed cardiac conditions and movement, while supporting a number of power-saving operating modes and transitions in-between such operating modes based on sensed data.

Electrocardiogram (ECG) signals detected by an ECG sensor are acquired, digitized and encoded at the sensor for communication to a device, either separate or co-located with the sensor for detecting arrhythmia using the encoded signal. The encoded version of the ECG signal for a single ECG period is significantly smaller compared to the digitized samples that represent the data acquired and digitized for the single ECG period. A wearable ECG sensor operates on a battery, and significant battery energy is saved at the ECG sensor since a compact representation if the ECG waveform is transmitted. Similarly, the parameters are efficiently extracted may be directly employed in arrythmia calculations. The disclosed technique permits a wearable device and a host device where the arrhythmia detection is performed to be used to detect arrhythmia is real-time.

The encoding technique at the ECG sensor encodes each period of the ECG waveform as locations for the extreme values of the Q, R, S, T and P waveforms relative to a temporal origin within the period, the respective extreme amplitudes and their widths at different fractions of the extreme amplitude and the period of the ECG signal for the arrhythmia detection. In lieu of the encoded information for the Q, R, S, T and P waves, a subset of the encoded waves (for example, only the encoding of the Q, R and S waves) can be communicated or passed to the arrhythmia detector. The detector, trained on similar encoding for known arrhythmic ECG waveform is able to classify the encoded waveform as normal or as belonging to a specific arrhythmia class. It is possible to use known machine learning techniques for arrhythmia detection.

Existing arrhythmia detectors require the use of all of the digitized samples for a complete ECG waveform or require a compressed version of the ECG waveform, all requiring more data to be communicated to the arrhythmia detector for the ECG sensor device. The present technology preferably encodes the ECG, and passes parameters of the ECG, while truncating the full waveform.

It is therefore an object to provide an electrocardiogram sensor, comprising: an input port configured to receive an electrocardiogramal; at least one automated processor, configured to: process a representation of the electrocardiogramal to determine an electrocardiographic waveform for a heartbeat cycle; and encode a set of quantitative parameters from the electrocardiographic waveform, dependent on a plurality of relationships within the electrocardiographic waveform comprising both amplitude-dependent features and time-dependent features of the electrocardiographic waveform; and a communication device, under control of the at least one automated processor, configured to communicate the encoded set of quantitative parameters through a communication channel.

It is a further object to provide a method of operating an electrocardiogram sensor, comprising: receiving an electrocardiogramal; processing a representation of the electrocardiogramal to determine an electrocardiographic waveform for a heartbeat cycle; automatically encoding a set of quantitative parameters from the electrocardiographic waveform, dependent on time and amplitude features within a graphical representation of the respective electrocardiographic waveform; and automatically controlling a communication of the encoded set of quantitative parameters through a wireless communication device.

It is also an object to provide a non-transitory computer readable medium storing instructions for controlling an electrocardiogram sensor, comprising: instructions for processing a representation of an electrocardiogramal to determine features of an electrocardiographic waveform for a heartbeat cycle; instructions for encoding a set of quantitative parameters from the electrocardiographic waveform, dependent on both feature amplitude relationships and feature time relationships of the electrocardiographic waveform for the heartbeat cycle; and instructions for controlling a communication of the encoded set of quantitative parameters through a wireless communication device.

The set of quantitative parameters may consist essentially of relative amplitude parameters, time duration parameters, and time difference parameters.

The electrocardiogram sensor may further comprise a memory configured to store a plurality of encoded sets of quantitative parameters, wherein the at least one automated processor is further configured to store the encoded set of quantitative parameters in the memory, and determine a type of an arrythmia from the encoded set of quantitative parameters for at least one heartbeat cycle, substantially without requiring the use of a stream of digitized values comprising the complete electrocardiogramal.

The electrocardiogram sensor may further comprise a receiver device, configured to receive the encoded set of quantitative parameters through the communication channel, and distinguish a type of an arrythmia represented in the electrocardiographic signal with respect to a plurality of arrythmia types, selected from the group consisting of at least two of Left Bundle Branch Block, Right Bundle Branch Block, Premature Ventricular Contraction, Atrial Premature Beat, and Paced Beat.

The electrocardiogram sensor may further comprise: an amplifier configured to amplify the electrocardiogramal; and a digitizer configured to create a digital representation of the electrocardiogramal, wherein the at least one automated processor is configured to: receive the digital representation as the representation of the electrocardiographic signal, determine a heart rate represented in the electrocardiographic waveform for the heartbeat cycle.

The at least one automated processor may be further configured to process the representation of the electrocardiographic signal to determine standardized electrocardiographic features, wherein the plurality of relationships within the electrocardiographic waveform comprising both amplitude-dependent features and time-dependent features of the electrocardiographic waveform are referenced to the determined standardized electrocardiographic features.

The at least one automated processor may be further configured to: determine a respective power consumption mode dependent on analysis of the representation of the electrocardiographic signal; and communicate different types of information packets through the communication channel containing respectively different information at different times selectively dependent on the determined respective power consumption mode.

The at least one automated processor may be further configured to: predict an ability of a communication receiver to receive and process the encoded set of quantitative parameters through the communication channel, and generate different types of information packets containing different information within the encoded set of quantitative parameters selectively dependent on the predicted ability of a communication receiver to receive and process the encoded set of quantitative parameters.

The at least one automated processor may be further configured to operate the electrocardiogram sensor in a plurality of different power consumption modes comprising respectively different duty cycles of operation of the communication device.

The at least one automated processor may be further configured to: receive a stream of digitized electrocardiographic data; maintain a data buffer storing the digitized electrocardiographic data; periodically produce the encoded set of quantitative parameters, for inclusion in a data packet for communication through the communication channel; and overwrite portions of the data buffer with subsequent digitized electrocardiographic data.

The electrocardiogram sensor may have a form factor comprising an adhesive flexible patch comprising electrocardiographic electrodes, the at least one automated processor, the communication device, and a battery configured to power the electrocardiogram sensor for at least 24 hours to continuously analyze the representation of the electrocardiographic signal, wherein the communication device comprises a Bluetooth or Bluetooth low energy wireless transceiver.

The electrocardiogram sensor may have a form factor comprising an adhesive flexible patch comprising electrocardiographic electrodes, the at least one automated processor, the communication device comprising a Bluetooth or Bluetooth low energy wireless transceiver, and a battery configured to power the electrocardiogram sensor.

The at least one automated processor may be further configured identify an R peak, QRS complex, P wave, and T wave within the electrocardiogramal, and to encode the set of quantitative parameters based on at least the identified R peak, QRS complex, P wave, and T wave.

The at least one automated processor may be configured to encode the set of quantitative parameters consisting essentially of a plurality of relative amplitude features representing differences in amplitude of a digitized electrocardiographic waveform and a plurality of temporal features representing latency or relative latency of features of the electrocardiographic waveform within a period of the heartbeat cycle. The latency or relative latency of features of the electrocardiographic waveform may be features which meet predetermined criteria dependent on the electrocardiographic waveform, such as maxima, minima, inflection points, ratiometrically-determined points, etc.

The at least one automated processor may be configured to encode the set of quantitative parameters:
The amplitude of Q, R, and S, peaks
Samples nearest predetermined proportions of R-peak along QR;
Distances between various peaks and defined samples.
For example:
1 Sampled highest signal amplitude, used as the R-peak;
2 Nearest sample at 80% of used R-peak (1) along QR;
3 Nearest sample at 60% of used R-peak (1) along QR;
4 Sampled amplitude of Q-peak;
5 Sample amplitude of S-peak;
6 Nearest sample at 60% of used R-peak (1) along RS;
7 Nearest sample at 80% of used R-peak (1) along RS;
8 Distance between samples used for (1) and (2);
9 Distance between samples used for (1) and (3);
10 Distance between samples used for Q- and R-peaks;
11 Distance between samples used for R- and S-peaks;
12 Distance between samples for Parameter 6 and R-peak; and
13 Distance between samples for Parameter 7 and R-peak.

The method may further comprise determining an existence and type of a cardiac arrythmia represented in the representation of the electrocardiographic signal dependent on the set of quantitative parameters.

The set of quantitative parameters may consists essentially of relative amplitude parameters, time duration parameters, and time difference parameters.

The method may further comprise: determining a respective power consumption mode dependent on at least one of: an analysis of the representation of the electrocardiographic signal; and a predicted ability of a communication receiver to receive and process the encoded set of quantitative parameters through the communication channel; and communicating at least one of different types of information packets containing respectively different information and different rates of information packets, through the communication channel at different times selectively dependent on the determined respective power consumption mode.

The may further comprise: storing digitized electrocardiographic data in a buffer memory; storing the encoded set of quantitative parameters for at least one heartbeat in the buffer memory; periodically communicating the encoded set of quantitative parameters for at least one heartbeat through the wireless communication device; communicating the stored digitized electrocardiogram the buffer memory through the wireless communication device based on a trigger; and overwriting portions of the buffer memory storing the communicated encoded set of quantitative parameters while selectively preserving portions of the buffer memory storing the encoded set of quantitative parameters which have not yet been communicated.

The method may further comprise identifying at least an R peak, QRS complex, P wave, and T wave within the electrocardiographic signal, and encoding the set of quantitative parameters based on at least the identified R peak, QRS complex, P wave, and T wave.

It is an object to provide an electrocardiogram sensor, comprising: an input port configured to receive an electrocardiographic signal; at least one automated processor, configured to: process a representation of the electrocardiogramal to determine an electrocardiographic waveform for a single heartbeat cycle; encode a set of quantitative parameters from the electrocardiographic waveform, dependent on a plurality of relationships within the electrocardiographic waveform comprising of amplitude-dependent features, pulse-width dependent features, and time-dependent features of a graphical representation of the electrocardiographic waveform; and control a communication of the encoded set of quantitative parameters; and a communication device, under control of the at least one automated processor, configured to communicate the encoded set of quantitative parameters through a communication channel. The set of quantitative parameters may consist essentially of amplitude, pulse width and time parameters.

The electrocardiogram sensor may have a form factor configured for use as a self-contained smart electrocardiographic wearable device, wherein the communication device comprises a wireless transceiver.

It is also an object to provide a method of operating an electrocardiogram sensor, comprising: receiving an electrocardiogramal; processing a representation of the electrocardiogramal to determine an electrocardiographic waveform for a single heartbeat cycle; encoding a set of quantitative parameters from the electrocardiographic waveform, dependent on time, and amplitude, and pulse width within a graphical representation of the respective electrocardiographic waveform; and controlling a communication of the encoded set of quantitative parameters through a wireless communication device, under control of at least one automated processor.

It is a further object to provide a non-transitory computer readable medium storing instructions for controlling an electrocardiogram sensor to perform various methods according to the invention, e.g., a method comprising: instructions for receiving an electrocardiogramal; instructions for processing a representation of the electrocardiogramal to determine an electrocardiographic waveform for a single heartbeat cycle; instructions for encoding a set of quantitative parameters from the electrocardiographic waveform, dependent on amplitude, time, and pulse width relationships of waveform features within a graphical representation of a respective electrocardiographic waveform; and instructions for controlling a communication of the encoded set of quantitative parameters through a wireless communication device, under control of at least one automated processor.

It is a further object to provide an electrocardiogram sensing system, comprising: an input port configured to receive an electrocardiographic signal; at least one automated processor, configured to: process a representation of the electrocardiogramal to determine an electrocardiographic waveform; and encode a set of quantitative parameters from a graphical representation of the electrocardiographic waveform, dependent on amplitude, width and relative spacing of components of the graphical representation of the electrocardiographic waveform; and a communication device, configured to communicate the encoded set of quantitative parameters through a communication channel substantially without communicating the electrocardiographic waveform itself. The communication device may comprise a radio frequency wireless communication device.

It is an object to provide an ECG sensor, comprising: an input port configured to receive an ECG signal; at least one automated processor, configured to: process a representation of the ECG signal to determine an ECG waveform for a single heartbeat; encode a set of quantitative parameters from the ECG waveform, dependent on a plurality of geometric relationships within the ECG waveform comprising relative amplitude-dependent features and relative time-dependent features; and control a communication of the encoded set of quantitative parameters; and a wireless communication device, under control of the at least one automated processor, configured to communicate the encoded set of quantitative parameters through radio frequency communication. The set of quantitative parameters may consist essentially of amplitude and time parameters. The at least one automated processor may be configured to determine a type of an arrythmia from the set of quantitative parameters substantially without reconstructing a model of the electrocardiographic signal.

A further object provides a method of operating an electrocardiogram sensor, comprising: receiving an electrocardiogramal; processing a representation of the electrocardiographic signal to determine an electrocardiographic waveform for a single heartbeat; encoding a set of quantitative parameters from the electrocardiographic waveform, dependent on geometric time and amplitude relationships within a respective electrocardiographic waveform; and controlling a communication of the encoded set of quantitative parameters through a wireless communication device, under control of at least one automated processor.

A still further object provides a non-transitory computer readable medium storing instructions for controlling an electrocardiogram sensor, comprising: instructions for receiving an electrocardiogramal; instructions for processing a representation of the electrocardiographic signal to determine an electrocardiographic waveform for a single heartbeat; instructions for encoding a set of quantitative parameters from the electrocardiographic waveform, dependent on amplitude and time relationships of waveform features within a respective electrocardiographic waveform; and instructions for controlling a communication of the encoded set of quantitative parameters through a wireless communication device, under control of at least one automated processor.

Another object provides an electrocardiogram sensing system, comprising: an input port configured to receive an electrocardiogramal; at least one automated processor, configured to: process a representation of the electrocardiographic signal to determine an electrocardiographic waveform; and encode a set of quantitative parameters from the electrocardiographic waveform, dependent on amplitude, width and relative spacing of components of the electrocardiographic waveform; and a wireless communication device, configured to communicate the encoded set of quantitative parameters substantially without communicating the electrocardiographic waveform itself.

It is an object to provide an electrocardiogram, comprising: an input port configured to receive an electrocardiogramal; at least one automated processor, configured to: process a representation of the electrocardiographic signal to determine an electrocardiographic waveform for a single heartbeat; encode a set of quantitative parameters from the electrocardiographic waveform, dependent on geometric relationships within the electrocardiographic waveform; and control a communication of the encoded set of quantitative parameters; and a wireless communication device, under control of the at least one automated processor, configured to communicate the encoded set of quantitative parameters through radio frequency communication.

It is a further object to provide a method of operating an electrocardiogram sensor, comprising: receiving an electrocardiogramal; processing a representation of the electrocardiogramal to determine an electrocardiographic waveform for a single heartbeat; encoding a set of quantitative parameters from the electrocardiographic waveform, dependent on geometric relationships within a respective electrocardiographic waveform; and controlling a communication of the encoded set of quantitative parameters through a wireless communication device, under control of at least one automated processor. A non-transitory computer readable medium is provided for storing instructions for controlling an electrocardiogram sensor to perform the method. The geometric relationships may be dependent on amplitude, width and relative spacing of components of the electrocardiographic waveform.

It is therefore an object to provide a wearable electrocardiogram sensor, comprising: an input port configured to receive an electrocardiogramal; at least one automated processor, configured to: process a representation of the electrocardiogramal to determine a QRS waveform for a single heartbeat; encode a set of quantitative parameters from the QRS waveform for the single heartbeat, dependent on amplitude relationships and latency relationships within a respective QRS waveform for the single heartbeat; and control a communication of the encoded set of quantitative parameters; and a wireless communication device, under control of the at least one automated processor, configured to communicate the encoded set of quantitative parameters through radio frequency communication.

It is a further object to provide a method of operating a wearable electrocardiogram sensor, comprising: receiving an electrocardiogramal; processing a representation of the electrocardiogramal to determine a QRS waveform for a single heartbeat; encoding a set of quantitative parameters from the QRS waveform for the single heartbeat, dependent on amplitude relationships and latency relationships within a respective QRS waveform for the single heartbeat; and controlling a communication of the encoded set of quantitative parameters through a wireless communication device, under control of at least one automated processor.

It is a still further object to provide a non-transitory computer readable medium storing instructions for controlling a wearable electrocardiogram sensor, comprising: instructions for receiving an electrocardiographic signal; instructions for processing a representation of the electrocardiogramal to determine a QRS waveform for a single heartbeat; instructions for encoding a set of quantitative parameters from the QRS waveform for the single heartbeat, dependent on amplitude relationships and latency relationships within a respective QRS waveform for the single heartbeat; and instructions for controlling a communication of the encoded set of quantitative parameters through a wireless communication device, under control of at least one automated processor.

The system, method and computer readable medium may provide that the at least one automated processor, or a host device communicating with the wearable electrocardiogram sensor, determine a cardiac arrythmia dependent on the set of quantitative parameters.

The system may further comprise a receiver device, configured to receive the encoded set of quantitative parameters through the radio frequency communication, and classify the electrocardiogramal as a cardiac arrhythmia to identify, and/or distinguish between at least two, three, or more of Left Bundle Branch Block, Right Bundle Branch Block, Premature Ventricular Contraction, and Atrial Premature Beat and optionally Paced Beat or other types of arrythmias.

The at least one automated processor may be further configured to classify the encoded set of quantitative parameters indicative of a cardiac arrhythmia, and to distinguish between at least two of Left Bundle Branch Block, Right Bundle Branch Block, Premature Ventricular Contraction, Atrial Premature Beat, and paced beat.

The system may further comprise an amplifier configured to amplify the electrocardiogramal; and a digitizer configured to create a digital representation of the electrocardiogramal, wherein the at least one automated processor is configured to receive the digital representation as the representation of the electrocardiogramal.

The at least one automated processor may be further configured to determine time periods of artifact, and suppress a communication by the wireless communication device during the determined periods of artifact.

The at least one automated processor may be further configured to determine a heart rate represented in the electrocardiographic signal.

The at least one automated processor may be further configured to determine a heart rate variability represented in the electrocardiographic signal.

The at least one automated processor may be further configured to selectively transmit the heart rate variability represented in the electrocardiogramal as a cumulative value over a series of heartbeats.

The at least one automated processor may be further configured to selectively transmit the heart rate variability represented in the electrocardiographic signal at predetermined intervals.

The at least one automated processor may be further configured to process the representation of the electrocardiogramal to determine electrocardiographic features.

The at least one automated processor may be further configured to generate information packets having a plurality of different information types, and excluding a stream of digitized samples of the electrocardiographic signal.

The at least one automated processor may be further configured to generate information packets according to different power consumption modes, wherein the at least one automated processor is configured to selectively determine a respective power consumption modes dependent on analysis of the representation of the electrocardiogramal.

The at least one automated processor may be further configured to determine a validity of data comprising the encoded set of quantitative parameters, and to suppress transmission of invalid data.

The at least one automated processor may be configured to communicate according to an error correcting protocol.

The at least one automated processor may be configured to communicate according to a reliable transmission protocol.

The at least one automated processor may be configured to encode the set of quantitative parameters (See FIG. 2):
1 Sampled highest signal amplitude, used as the R-peak;
2 Nearest sample at 80% of used R-peak (1) along QR;
3 Nearest sample at 60% of used R-peak (1) along QR;
4 Sampled amplitude of Q-peak;
5 Sample amplitude of S-peak;
6 Nearest sample at 60% of used R-peak (1) along RS;
7 Nearest sample at 80% of used R-peak (1) along RS;
8 Distance between samples used for (1) and (2);
9 Distance between samples used for (1) and (3);
10 Distance between samples used for Q- and R-peaks;
11 Distance between samples used for R- and S-peaks;
12 Distance between samples for Parameter 6 and R-peak; and
13 Distance between samples for Parameter 7 and R-peak.

An additional (14^{th}) parameter is the pulse rate, though heart rate and heart rate variability may be calculated by the device receiving the packets.

The wearable electrocardiogram sensor may further comprise a self-contained power source, configured to power the at least one automated processor and the wireless communication device.

The wearable electrocardiogram sensor may further comprise a flexible substrate having at least two electrodes formed on the flexible substrate, configured to receive transdermal bioelectric signals representing the electrocardiographic signal. The at least two electrodes may comprise a gold surface.

The at least one automated processor may be further configured to determine a heart rate, and to include the heart rate with the encoded set of quantitative parameters in a packet for digital transmission.

The at least one automated processor may be configured to: receive a stream of digitized electrocardiographic data; maintain a data buffer storing the digitized electrocardiographic data; periodically encode the set of quantitative parameters, for inclusion in the packet; and overwrite the contents of the data buffer with subsequent digitized electrocardiographic data.

The wearable electrocardiogram sensor may be configured as a self-contained wearable smart ECG wearable device.

The wireless communication device may be compliant with IEEE-802.15, BLE, Bluetooth, WFi (IEEE-802.11 family of protocols), ANT, LoPAN transceiver, Zigbee, LTE, 5G, 6G, or the like.

The wireless communication device may be configured to communicate over an encrypted data communication channel, an error detecting protocol, an error detecting and correcting (EDC) protocol, a reliable protocol, or the like.

The at least one automated processor may be further configured to determine an electrocardiographic alarm state, and to transmit an alert selectively in dependent on the determined electrocardiographic alarm state.

The at least one automated processor may be further configured to receive control information which determines an energy consumption rate through the wireless communication device.

The at least one automated processor may be further configured to transmit information related to a state of a power source through the wireless communication device.

The wearable electrocardiogram sensor may further comprise: a substrate comprising a flexible substrate having at least two electrodes adapted for skin contact, configured to receive transdermal bioelectric signals representing the electrocardiographic signal; and an adhesive layer configured to adhere with flexible substrate to human skin, without interference with the at least two electrodes making skin contact. The substrate may comprise a flexible substrate configured to support packed integrated circuits. The substrate may comprise a flexible substrate configured to support bare die integrated circuits. The substrate may have an antenna printed thereon.

The periods of artifact may comprise periods of noise artifacts, motion artifacts, periods of electrode non-contact artifacts, external interference artifacts, or the like.

The at least one automated processor may be further configured to filter artifacts, to reduce occurrence of periods of artifact.

An analog filter may be provided to filter artifacts, to reduce occurrence of periods of artifact.

The at least one automated processor, digitizer, and wireless communication device may be integrated within a single integrated circuit.

A remote host may be provided, configured to: communicate with the wireless communication device; receive the information packets; and transmit control information to at least alter a power consumption of the wireless communication device.

The at least one automated processor may be configured to analyze the determined electrocardiographic features to determine at least heart rate by determining occurrence of R waves.

The at least one automated processor may be further configured to: process the digital representation to determine a baseline; determine a leads off condition of an electrocardiographic electrode; and redetermine the baseline after the leads off condition has abated.

The at least one automated processor may be further configured to determine occurrence of a cardiac arrhythmia.

The at least one automated processor may be configured to control the wireless communication device to generate an alarm upon the determined occurrence of the cardiac arrhythmia.

The at least one automated processor may be configured to receive a signal from a local host through the wireless communication device upon determination by the local host of a cardiac arrythmia, and to change an operating mode of the wearable electrocardiogram sensor in dependence on the determined cardiac arrythmia.

The at least one automated processor may be configured to operate in a plurality of power consumption modes comprising a sleep mode and an awake mode.

The at least one automated processor may be further configured to change between a sleep mode and an awake mode in a duty cycle.

It is an object to provide a wearable electrocardiogram sensor patch, comprising: an amplifier configured to amplify a signal comprising cardiac electrical activity; a digitizer configured to create a digital representation of the signal; a microprocessor, configured to: receive the digital representation of the signal; process the digital representation to determine at least one electrocardiographic features and periods of artifact during which cardiac electrical activity is unavailable; analyze the determined at least one electrocardiographic feature to determine at least heart rate; adaptively generate information packets having a plurality of different information types, the information packets having a respective information type dependent on at least the determined at least one electrocardiographic feature and the determined periods of artifact; and a wireless communication device, under control of the microprocessor, configured to remain in a non-transmitting low power state without transmitting information packets for the periods of artifact, and to enter a transmitting high power state for transmission of the adaptively generated information packets containing valid statistics for at least the heart rate, and subsequently revert to the low power state after transmission.

It is also an object to provide a method for operating wearable electrocardiogram sensor patch, comprising: amplifying a signal comprising cardiac electrical activity received from a set of electrodes with an amplifier; digitizing the amplified signal to create a digital representation of the signal; automatically processing the digital representation to determine at least one electrocardiographic feature and periods of artifact during which cardiac electrical activity is unavailable; analyzing the at least one determined electrocardiographic feature to determine at least heart rate; adaptively generating information packets having a plurality of different information types, the information packets having a respective information type dependent on at least the determined at least one electrocardiographic feature and the determined periods of artifact; and wirelessly communicating the information packets, under control of a microprocessor, to remain in a non-transmitting low power state without transmitting information packets for the periods of artifact, and to enter a transmitting high power state for transmission of the adaptively generated information packets containing valid statistics for at least the heart rate, and subsequently revert to the low power state after transmission.

The wirelessly communicating may comprise transmitting a radio frequency signal through an antenna formed on a flexible substrate supporting the microprocessor and at least two of the set of electrodes configured to acquire an electrocardiogramal through human skin; further comprising: receiving a command through the antenna to control the microprocessor; buffering electrocardiographic with the microprocessor in a memory, periodically forming an information packet from the buffered electrocardiographic data; and overwriting the buffered electrocardiographic data after mission. The wireless communication may be radio frequency (Bluetooth/BLE, WiFi, 3G, 4G, 5G, 6G), and may alternately be light (e.g., infrared, visible light, ultraviolet), acoustic (ultrasonic, audible), backscatter optical or radio frequency communications (e.g., RFID standards EPC UHF Gen2v2 or ISO/IEC 18000, ISO 14443, ISO 15693, NFC), or the like.

The method may further comprise extracting a plurality of electrocardiographic features from the cardiac electrical activity; and transmitting at least one of the plurality of electrocardiographic features and a series of samples from the digitizer in the adaptively generated information packets selectively in dependence on said automatic processing.

The method may further comprise determining an electrocardiographic alarm state based on said automatic processing selectively during periods without artifact, and communicating an alert selectively in dependence on the determined electrocardiographic alarm state.

It is a further object to provide a non-transitory computer readable medium for controlling at least one microprocessor operating a wearable electrocardiogram sensor patch, comprising: instructions for digitizing a signal from a set of electrodes to create a digital representation of the signal; instructions for processing the digital representation to determine at least one electrocardiographic feature and periods of artifact during which cardiac electrical activity is unavailable; instructions for analyzing the at least one determined electrocardiographic feature to determine at least heart rate; instructions for adaptively generating information packets having a plurality of different information types, the information packets having a respective information type dependent on at least the determined at least one electrocardiographic feature and the determined periods of artifact; and instructions for communicating the information packets, to remain in a non-transmitting state without transmitting information packets for the periods of artifact, and to enter a transmitting state for transmission of the adaptively generated information packets containing valid statistics for at least the heart rate, and subsequently revert to the non-transmitting state after transmission.

A substrate may be provided for supporting the microprocessor, a set of electrodes for receiving the signal comprising cardiac electrical activity, and a self-contained power source, configured to power the amplifier, the digitizer, the microprocessor, and the wireless communication device. The substrate may be a flexible substrate having at least two of the set of electrodes formed on the flexible substrate, configured to receive the transdermal bioelectric signals representing the cardiac electrical activity through human skin; and an adhesive layer configured to adhere with flexible substrate to human skin, without interference with the set electrodes.

The microprocessor may be further configured to determine heart rate variability, and the adaptively generated information packets further contain valid statistics for at least the heart rate variability. The microprocessor may be configured to: maintain a data buffer for electrocardiographic data in a memory, periodically form an information packet from the maintained electrocardiographic data; and overwrite the contents of the data buffer with subsequent electrocardiographic data. The microprocessor may be configured to extract a plurality of electrocardiographic features from the cardiac electrical activity, and transmit at least one of the plurality of electrocardiographic features and a series of samples from the digitizer representing the cardiac electrical activity in the adaptively generated information packets. The microprocessor may be further configured to determine an electrocardiogram state, and to transmit an alert selectively dependent on the determined electrocardiographic alarm state. The microprocessor may be further configured to receive control information through the wireless communication device which determines an energy consumption rate of the wearable electrocardiogram sensor patch.

The microprocessor may be configured to analyze the determined at least one electrocardiographic determine at least heart rate by determining occurrence of R waves.

The microprocessor may be further configured to automatically: process the digital representation to determine a baseline; determine a leads-off condition of an electrocardiographic electrode; and redetermine the baseline after the leads off condition has abated.

The microprocessor may be further configured to determine occurrence of a cardiac arrhythmia, and to selectively adaptively generate the information packets comprising information indicative of the cardiac arrythmia.

The microprocessor may be further configured to control the wireless communication device to communicate the digital representation subsequent to the determined occurrence of the cardiac arrhythmia, and to control the wireless communication device to communicate a set of parameters characterizing the cardiac electrical activity but not comprising the digital representation prior to the determined occurrence of the cardiac arrhythmia.

A microprocessor-implemented biological model of expected cardiac electrical activity may be maintained in the SEP, and wherein the periods of artifact represent periods during which the digital representation of the electrocardiographic signal includes unexpected values with respect to the microprocessor implemented biological model.

The substrate may comprise a flexible substrate having at least two electrodes adapted for skin contact, configured to receive the transdermal bioelectric signals representing the cardiac electrical activity, further comprising an adhesive layer configured to adhere with flexible substrate to human skin, without interference with the at least two electrodes making skin contact.

The periods of artifact may comprise periods of at least one of noise artifacts, motion artifacts, and electrode non-contact artifacts.

It is also an object to provide a method for operating a wearable electrocardiogram sensor patch, comprising: providing a patch having an amplifier configured to amplify an electrocardiogramal representing cardiac electrical activity, a digitizer configured to create a digital representation of the electrocardiographic signal, a microprocessor, a radio frequency transceiver having an antenna, and a self-contained power source, configured to power the amplifier, the digitizer, the microprocessor, and the radio frequency transceiver; receiving the digital representation of the electrocardiographic signal; determining periods of artifact; processing the digital representation to determine electrocardiographic features; analyzing the determined electrocardiographic features to determine at least heart rate; adaptively generating information packets having a plurality of different information types, the information packets having a respective information type dependent on at least the determined electrocardiographic features and the determined periods of artifact; and transmitting the adaptively generated information packets containing valid statistics for at least the heart rate, and remaining in a non-transmitting low power state without transmitting information packets for the determined periods of artifact.

It is also an object to provide a non-transitory computer readable medium for controlling at least one microprocessor operating a wearable electrocardiogram sensor patch, comprising: instructions for receiving a digital representation of the electrocardiogramal from a digitizer; instructions for processing the digital representation to determine a baseline; instructions for determining a need to redetermine the baseline based on artifacts within the digital representation of the electrocardiogramal; instructions for processing the digital representation to determine electrocardiographic features; instructions for analyzing the determined electrocardiographic features to determine at least heart rate; instructions for adaptively generating information packets having a plurality of different information types, the information packets having a respective information type dependent on at least the determined electrocardiographic features and a deviance of the digital representation of the electrocardiographic signal from the baseline; and instructions for controlling a transceiver to transmit the adaptively generated information packets containing valid statistics for at least the heart rate, and remaining in a non-transmitting low power state without transmitting information packets for periods of artifact.

It is a further object to provide a non-transitory computer readable medium for controlling at least one microprocessor operating a wearable electrocardiogram sensor patch, comprising: instructions for receiving a digital representation of the electrocardiogramal from a digitizer; instructions for processing the digital representation to determine digital representations representing artifact; instructions for processing the digital representation to determine electrocardiographic features; instructions for analyzing the determined electrocardiographic features to determine at least heart rate; instructions for adaptively generating information packets having a plurality of different information types, the information packets having a respective information type dependent on at least the determined electrocardiographic feature and the determined digital representations representing artifact; and instructions for controlling a transceiver to transmit the adaptively generated information packets containing valid statistics for at least the heart rate, and remaining in a non-transmitting low power state without transmitting information packets having information corrupted by the determine digital representations representing artifact.

A further object provides a wearable electrocardiogram sensor patch, having a substrate comprising: an amplifier configured to amplify an electrocardiogramal representing cardiac electrical activity; a digitizer configured to create a digital representation of the electrocardiographic signal; a microprocessor, configured to: receive the digital representation of the electrocardiogramal, determine periods of artifact, process the digital representation to determine electrocardiographic features, analyze the determined electrocardiographic features to determine at least heart rate, and adaptively generate information packets having a plurality of different information types, the information packets having a respective information type dependent on at least the determined electrocardiographic feature and the determined periods of artifact; a radio frequency transceiver having an antenna, under control of the microprocessor, configured to transmit the adaptively generated information packets containing valid statistics for at least the heart rate, and to remain in a non-transmitting low power state without transmitting information packets for the determined periods of artifact; and a self-contained power source, configured to power the amplifier, the digitizer, the microprocessor, and the radio frequency transceiver.

The wearable electrocardiogram sensor patch may be configured as a self-contained wearable smart ECG wearable device.

The substrate may be a flexible substrate having at least two electrodes formed on the flexible substrate, configured to receive the transdermal bioelectric signals representing the cardiac electrical activity. The at least two electrodes may comprise a gold surface. The substrate may comprise a flexible substrate having at least two electrodes adapted for skin contact, configured to receive the transdermal bioelectric signals representing the cardiac electrical activity, further comprising an adhesive layer configured to adhere with flexible substrate to human skin, without interference with the at least two electrodes making skin contact. The substrate may comprise a flexible substrate configured to support packed integrated circuits and/or bare die integrated circuits. A printed antenna may be provided on the flexible substrate.

The transceiver may be compliant with IEEE-802.15, or be a BLE, Bluetooth, ANT, or a LoPAN transceiver. While not preferred due to power consumption and generally excess capabilities, WiFi may be supported. The transceiver may be multi-protocol. The transceiver may communicate over an encrypted data communication channel.

The microprocessor may be further configured to determine heart rate variability, and the adaptively generated information packets may further contain valid statistics for at least the heart rate variability. The microprocessor may be configured to maintain a data buffer for maintaining electrocardiogram memory, to periodically form an information packet from the maintained electrocardiographic data, and to overwrite the contents of the data buffer with subsequent electrocardiographic data. The microprocessor may be further configured to determine an electrocardiographic alarm state, and to transmit an alert selectively in dependence on the determined electrocardiogram state. The microprocessor may be further configured to receive control information which determines an energy consumption rate through the transceiver. The microprocessor may be further configured to transmit information related to a state of the self-contained power source through the transceiver.

The microprocessor may be further configured to: process the digital representation to determine a baseline; determine a leads-off condition of an electrocardiographic electrode; and redetermine the baseline after the leads-off condition has abated.

The microprocessor may be configured to analyze the determined electrocardiographic features to determine at least heart rate by determining occurrence of R waves.

The microprocessor may be further configured to determine occurrence of a cardiac arrhythmia. The microprocessor may be configured to control the transceiver to generate an alarm upon the determined occurrence of the cardiac arrhythmia The microprocessor may be configured to control the transceiver to generate an alarm signal to a local host upon determined occurrence of the cardiac arrhythmia, and to control the host to transmit a corresponding alarm to a remote server upon receipt of the alarm signal.

The microprocessor may have a plurality of power consumption modes comprising a sleep mode and an awake mode, and wherein the microprocessor is programmed to alternate between sleep mode and awake mode in a duty cycle.

The periods of artifact comprise periods of noise artifacts and/or periods of motion artifacts. These artifacts may, in some cases, be suppressed by analog and/or digital filtering, and if suppressed, the electrocardiogramal processed to determine features during periods of suppressed artifacts. However, if the artifacts are not reliably suppressed, the microcontroller preferably does not calculate cardiac statistics perturbed by the artifacts. The electrocardiographic signal may be received through at least one electrode, and the periods of artifact comprise periods of electrode non-contact artifacts. The microprocessor may be further configured to filter artifacts, to reduce occurrence of periods of artifact.

The wearable electrocardiogram sensor patch may further comprise an analog filter configured to filter artifacts, to reduce occurrence of periods of artifact.

At least the microprocessor, digitizer, and transceiver (i.e., the digital circuitry) may be integrated within a single integrated circuit.

The wearable electrocardiogram sensor patch may communicate with a remote host configured to communicate with the transceiver, to receive the information packets, and transmit control information to at least alter the power consumption of the transceiver.

The technology may reduce power wastage due to transmission of incorrect human subject's data by preprocessing raw acquired data from the sensor, followed by the suppression of data transmission if the acquired data is not in compliance with what is expected, with the expectation being defined as signal parameters remaining within limits that are prespecified.

The technology may reduce power wastage due to transmission of incorrect human subject data by incorporating algorithms to preprocess the raw acquired data from the sensor followed by the suppression of data transmission if the acquired data is not in compliance with what is expected, with the expectation being defined as signal parameters remaining within limits that are derived from historical measurements during device operation. The failure to meet expectation(s) may be considered an artifact. The expectation may alternately be defined as signal parameters remaining within limits that are derived from clinical data pertaining to the specific subject. The expectations may be generated within the SEP, or communicated to it by the host. The limits may be determined adaptively, and for example, may be responsive to activity level, diurnal variation, past history, variability or other statistical properties of prior readings, etc.

Expected signal characteristics and artifacts (i.e., readings whose value does not accurately reflect the biological process being monitored) may be distinguished by signals being out of range, or by patterns which are either similar to known interference patterns, or dissimilar from biological process patterns, or both. Given the typically limited processing power of the locally-executed algorithm(s), upon initial presentation, the SEP may transmit the possibly artefactual data to the host, wherein the host analyzes the signal, and makes a determination, and thereafter communicates an updated profile or algorithm to the SEP to permit reliable filtering. In most cases, the types of artifacts and interference are established in a predetermined manner, and therefore the algorithm executing in the SEP need not be updated. However, in some cases, new types of interference or artifact may emerge.

The SEP may tokenize certain types of data or messages, and intermittently transmit only small messages indicative of its state. These may take the form of a heartbeat message, which advantageously includes a power source state. For example, in many cases, the ECG pattern may be regular, and only the statistics, e.g., heart rate and heart rate variability are transmitted, e.g., every 5 minutes. However, when an arrhythmia is detected, which may be determined by an aberrant ECG waveform and/or interbeat latency which is of an unexpected value, then the transmission may be converted to a real-time ECG stream. In this case, “real-time” means that all of the data after appropriate filtering is transmitted, which may be as intermitted packets through the digital packet radio, if the packet data transmission rate exceeds the data acquisition rate, or a stream of packets that may extend beyond the time of the reading if the packet data transmission rate is lower than the data acquisition rate. In any case, a duration of readings, e.g., 60 seconds or 300 seconds, is recorded and transmitted to the host, and the host may then analyze the readings and/or forward them to a remote server or processing center.

The communication between the SEP and host is typically encrypted. In some cases, the SEP may engage in a virtual private network communication with a remote server or center, permitting the local host to be untrusted. See, U.S. Pat. Nos. 10,305,695; 9,942,051; and 9,215,075, and references incorporated therein.

### BRIEF DESCRIPTION OF THE DRAWINGS

FIG. 1 shows an exposed SEP prototype, top view.

FIG. 2 shows 13 parameters extracted from the QRS complex of ECG for encoding ECG.

FIG. 3 shows the steps involved in the acquisition, analysis, ECG signal encoding and transmission on the SEP sensor and processing steps on the host-side device. These steps, forming a cycle, run continuously.

FIG. 4 shows bidirectional LSTM Model for ACES. Output layer tensor shapes are shown in parentheses.

FIG. 5A shows training and validation loss over number of epochs.

FIG. 5B shows training and validation AUC measure over number of epochs.

FIG. 5C shows a confusion plot of ACES arrhythmia multiclass classifier using Test dataset. Arrhythmia class 1-class 6 represents arrhythmias from Table II.

FIG. 6 shows a current drawn during Encoded ECG Transmission versus Current drawn during full ECG Transmission, when SEP is transmitting continuously (100% duty cycle).

FIG. 7A shows a high-level diagram of functions of SEP and a Bluetooth host.

FIG. 7B shows a high-level diagram of modules of the SEP and host.

FIG. 8 shows an abstract representation of ECG signal of one period.

FIG. 9A shows a leads-off detection scenario.

FIG. 9B shows noise/Motion Artifact detection scenario. Circles show the aberrations detected.

FIG. 10A shows an MIT-BIH ECG signal. R-peak detection, and Heart rate calculation are performed onboard the SEP, processed on MIT-100 ECG signal.

FIG. 10B shows a human subject ECG signal. R-peak detection, and Heart rate calculation are performed onboard the SEP, processed on the ECG signal from Human Subject. The y-axis on the left represents the digitized ECG signal amplitude. The y-axis to the right represents the instantaneous heart rate (HR) (dots). R-peaks are detected and represented by a triangular marker.

FIGS. 11A-11D show comparisons of four iterations of heart rate variability data computed by SEP (left bar) against data computed by Shimmer (middle bar), and BioSPPy (right bar).

FIG. 12A shows both ECG and heart rate transmissions and power consumed by SEP in a Simple Duty cycling seen on a Bluetooth host device during an active period. heart rate is marked with dots.

FIG. 12B shows the electrical current drawn by SEP operating in Simple Duty cycling mode at a 10% duty cycle.

FIG. 13A shows heart rate and ECG signals transmissions and power consumed as seen on Bluetooth host device during an active period in AD-CHR+TE power mode. Heart rate is marked with dots.

FIG. 13B shows the electrical current drawn by SEP operating in AD-CHR+TE power mode at a 10% duty cycle.

FIG. 14A shows ECG and heart rate transmissions and power consumed in AD-TR+TE power mode as seen on host device interface during an active period. Heart rate is marked with dots.

FIG. 14B shows the electrical current drawn by SEP in AD-TR+TE power mode at 10% duty cycle.

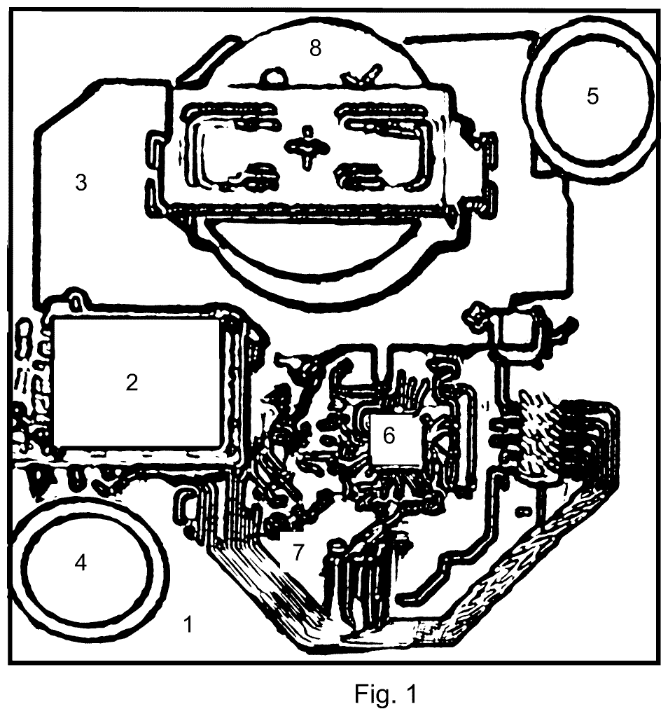

### DETAILED DESCRIPTION OF THE PREFERRED EMBODIMENTS

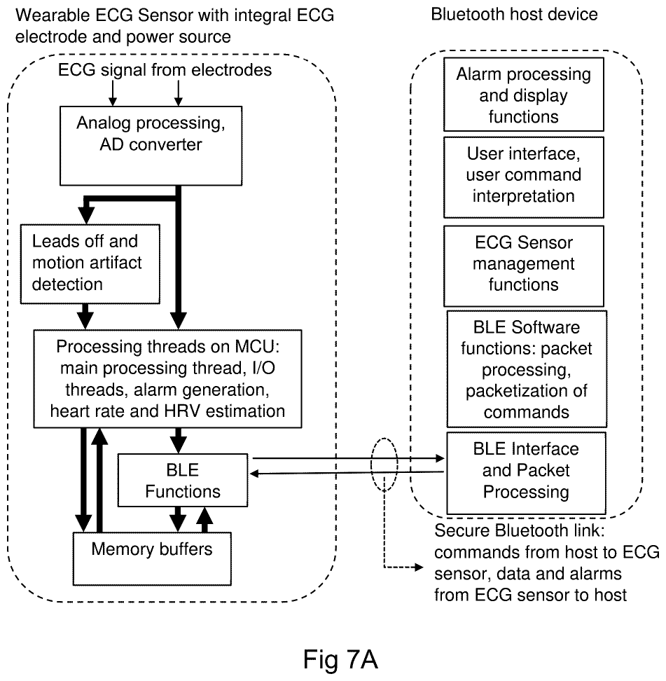

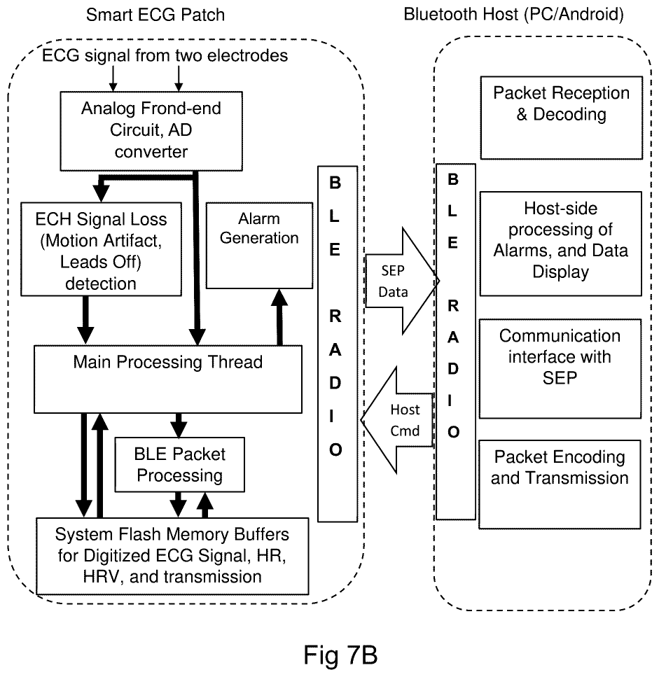

The overall architecture of the system is shown in FIG. 7A along with the main software modules of SEP and host shown in FIG. 7B. The ECG signal is acquired using a 2-lead configuration where the electrodes are located at the two diagonal ends of the patch on the body side to get the maximum possible spacing between the electrodes and to get the highest possible ECG signal amplitude under the dimensional constraints of the patch. The ECG signal is processed and cleaned up using analog front-end circuitry that filters out signal outside the frequency band of interest using a multi-pole filter. This front-end circuitry also limits current flow into the human subject going through the electrodes in the worst case, if circuit damage applies the full battery voltage across the electrodes.

The rest of the data acquisition module, Analog to Digital Converter (ADC), signal cleanup, buffering, transmission to a Bluetooth (en.wikipedia.org/wiki/Bluetooth) or BLE (en.wikipedia.org/wiki/Bluetooth_Low_Energy) host device is implemented by a micro controller unit (MCU) module with a radio frequency transceiver. A number of suitable processors are available, including TI CC2640, nRF52832, MSP430, TI CC2530 system CC2431, TMS320VC5509A TMS320F2812, TMDX5505cZDsp/VC5505eZdsp, MSP430F5515, MSP430F1232, MSP430FG439, MSP430F2418 MSP430F5529, MSP430F5419A, ATmega8, ATmega328, PIC18LF4620, ADuC842, ESP32, and ESP8266. These may be paired with, for example an analog front end (AFE) such as a TI AFE49130 (ECG with pulse oximeter) or ADS 129X (ECG only).

A Real-time Operating System (RTOS) and available libraries provide the main scheduling and support functions. For example, the CC2640 device is a wireless MCU targeting Bluetooth Low Energy applications. The CC2640 device contains a 32-bit ARM Cortex-M3 processor that runs at 48 MHz as the main processor and a variety of peripherals that include an ultralow power sensor controller. This sensor controller interfaces external sensors and for collecting analog and digital data autonomously while the rest of the system is in sleep mode. An ARM Cortex-MO is provided as an RF system controller.

Several software modules perform the functions of the SEP, with each module being executed in a thread. The thread for one process interacts with other thread(s) used in SEP's software via synchronization variables and buffers in the RAM. (i) The Data Acquisition Thread (DAT) controls all aspects of data acquisition, its digitization and the storage of the digitized signal within a buffer within the MCU's RAM. A 12-bit ADC (analog-to-digital converter), operated at a 400 Hz sampling rate, is used. (ii) The Main Processing Thread (MPT) examines the digitized data in the buffer, performs R-peak detection and calculates the heart rate and heart rate variability, perform noise and motion artifact removal by signaling the Artifact Detection Thread (ADT) (discussed below) and performs transmission checks as dictated by the operating mode. (iii) An analysis and diagnostics thread calculates the HR and HRV and for the MPT. (iv) A transmission thread is responsible for converting the cleaned-up, digitized signals into BLE packets and transmitting it to the host as needed. (v) A receive thread is responsible for receiving command packets from the Bluetooth/BLE host and for interpreting the commands, which are then passed on to the MPT. If data transmission to the host is warranted, the MPT deposits the HR, HRV and/or the digitized ECG signal into the transmission buffer for access by the transmission thread.

The MPT also performs transmission checks as dictated by the power saving modes selected from the host device to prolong battery life. An operating mode can also be changed dynamically, for example, based on alarm signals derived from the ECG waveform by the SEP's noise and motion Artifact Detection Thread (ADT), or based on low battery voltage, by the SEP or the host device. An operating mode can also be changed dynamically, for example, based on alarm signals derived from the ECG waveform by the sensor's MPT thread or based on the battery voltage or based on commands from the host. SEP's operating modes permit power conservation to increase the operating time of a limited energy source, such as a lithium coin cell battery. Data sent to the host from SEP can be analyzed on the host, or further transmitted to a remote server or the “cloud” for personalized diagnostics, as in the system of (De Capua et al. 2010).

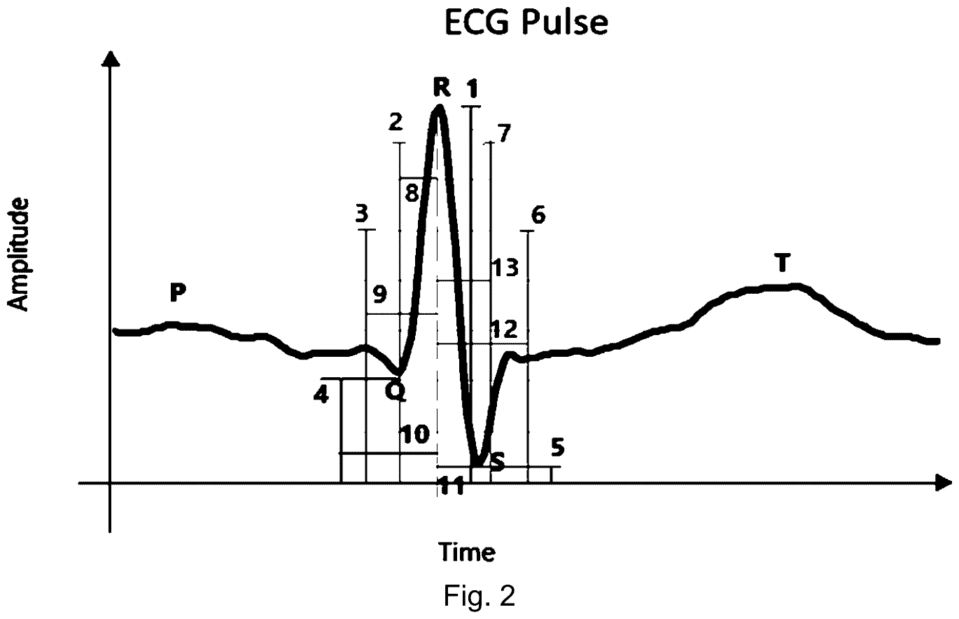

The SEP also may operate to extract parameters of the ECG signal, and transmit these in real time to the Bluetooth/BLE host. FIG. 2 shows the basis for 13 distinct parameters that may be quantified from each QRS waveform in the ECG signal. These may be transmitted for each heartbeat, or aggregated over a short period and communicated as a group, for example, four heartbeats. The transmission strategy may be adaptive, and therefore when the QRS complex has consistent intervals and values, the maximum transmission latency may be increased, while when the intervals between QRS are uneven or changing, and/or all or a portion of the 13 parameters change or are outside predetermine or adaptive limits, the maximum latency may decrease, and for example, the packet may be sent for each heartbeat immediately after calculation. In some cases, the SEP can revert to a mode in which the raw or minimally processed ECG signal is transmitted or streamed to the host device.

The SEP may also have a mode in which it operates in a stand-alone or hostless mode, and emits an alarm signal, such as a sound through a piezoelectric bender or speaker, transmits at alarm or message through Bluetooth, BLE, WiFi, LTE, 5G, etc., or other protocol. The SEP may be paired with a smartwatch (e.g., Samsung Galaxy Watch 5 under Android Wear OS 2.5) as host, which advantageously provides its own sensors, such as motion, photoplethysmography (PPG), etc., as well as processing and remote communications.

When data transmission to the host is warranted, such as per heartbeat, per group of heartbeats, on alarm condition, or other trigger, the MPT stores the ECG parameters, and e.g., heart rate, heart rate variability, and/or the digitized ECG signal itself into the transmission buffer for access by the transmission thread. The SEP connects to a Bluetooth Low Energy host using a Bluetooth Low Energy (BLE) protocol to transmit the information in the buffer, e.g., ECG signals, ECG encoded parameters, heart rate, heart rate variability, alarms, and other configuration/system data. A Transmit Thread (TT) encapsulates packet payloads in the buffer into BLE packets and transmits it to the host as needed. A Receive Thread (RT) on the SEP receives command packets from the host and interprets such commands or otherwise acts on them.

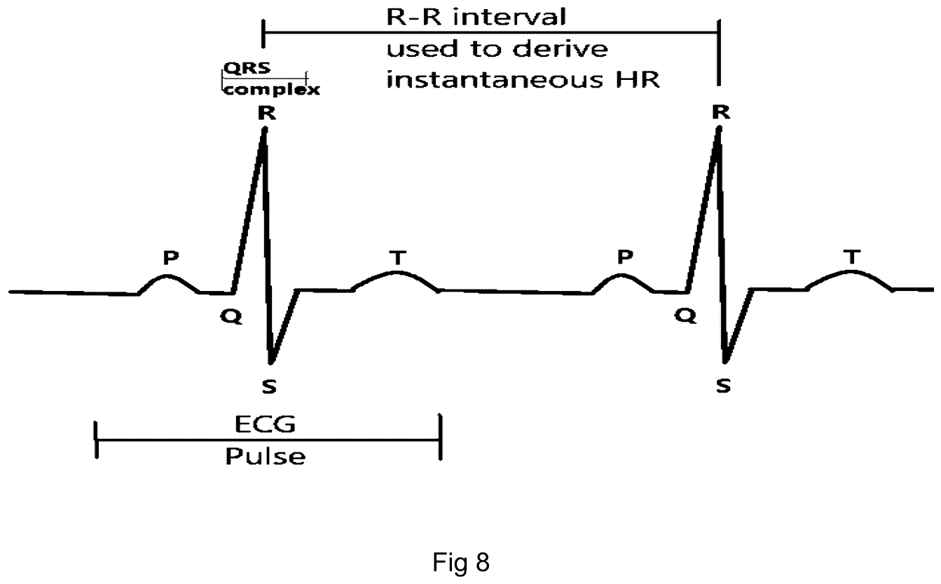

An ECG signal is a complex signal consisting of three temporally separated waveforms of interest, namely, P, QRS, and T, as shown in an abstract form FIG. 8. The SEP processes real-time ECG signals to calculate the heart rate (HR), and heart rate variability (heart rate variability) parameters by detecting the R-peaks in the QRS complex wave. The period between consecutive R-peaks in the acquired data determines the heart rate, which is the number of R peaks occurring within a period of time, e.g., one minute or one second. The onboard software processes the digitized signal and detects the R-Peaks by sensing the slope changes and the relative amplitude of the signal in relation to other peaks in the ECG waveform.

The R-peak amplitude, as sensed across the electrodes, is typically less than 5 mV. Analog circuitry, e.g., an instrumentation amplifier, amplifies this signal, removes higher frequency components and any 60-Hz pickup from the analog signal using multi-pole filters. The digitized output from the ADC is a 12-bit or 16-bit unsigned integer ranging between 0 and 4095 and is stored in the RAM. A value within this range represents the digitized value of each ECG signal sample.

As noted above, the MPT, or a subtask determines the QRS waveform complex, and extracts a number of parameters characterizing the complex. These parameters advantageously provide the key information for analyzing the QRS waveform complex, and therefore alleviate in most cases the need to transmit the raw data. Further, the amount of data required is significantly reduced. The allocation of bits in a packet between the various encoded parameters may be static or adaptive, with optional delta encoding, which can be particularly efficient if the SEP and the Host adapt based on the same information and algorithm, and therefore avoid the need for transmission of additional decoding information from the SEP to the host. In an adaptive or delta/predictive encoding data transmission system, it is preferred that periodically, full information is communicated to reset the receiver in case of corruption. An error detection and correction and/or reliable protocol may be implemented, though this may not be required in most instances, and would typically increase power consumption (unless the transmit power is generally reduced to a low level with an acceptable modest error rate, which is corrected based on the protocol, as compared to a non-feedback/error corrected protocol, in which greater transmit power may be required to reliably obtain a sufficiently low error rate under various circumstances).

When first turned on, the thread MPT determines the presence of any input ECG signal using the assistance of the thread ADT before performing any heart rate, heart rate variability calculation, or ECG QRS parameter extraction. The MPT determines the baseline reference of the signal in real-time. The initial baseline is the running average of the ECG signal, excluding the R and S peaks. To do this, the SEP is put on the subject and used to acquire a clean signal with the subject at rest. The initial baseline value is calculated from this signal. During this initialization phase, each incoming ECG sample is buffered into the memory for e.g., two seconds. Thereafter, the baseline is recalculated continuously to detect motion artifacts by rejecting any high frequency peaks below a specific amplitude range on either side of the current baseline, as described later. The rejected signals are considered as noise.

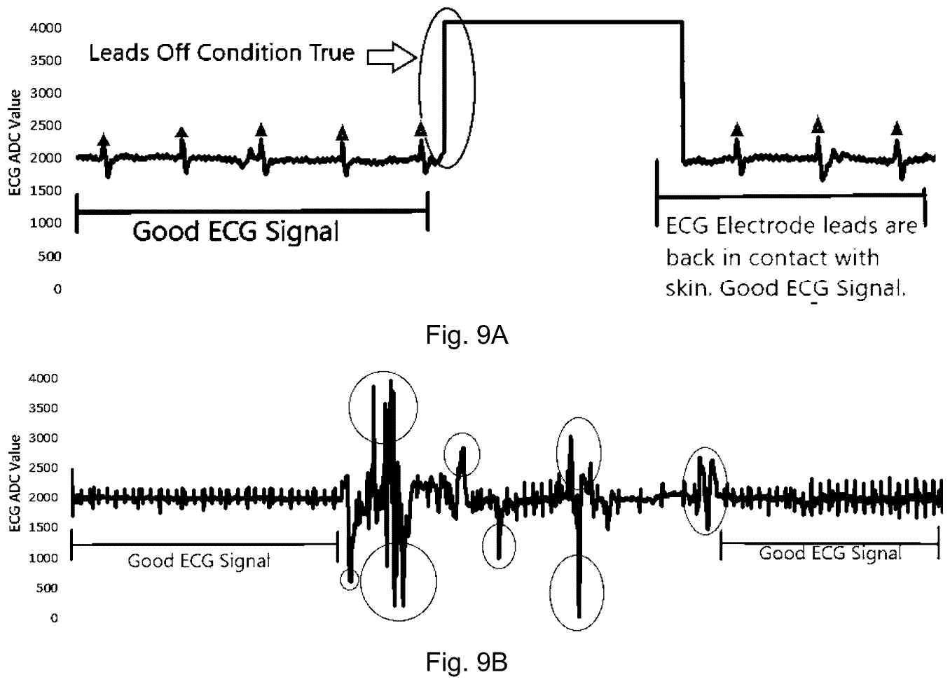

A wearable ECG sensor like SEP can flex during use as the wearer moves and this can lead to two types of signal artifacts: (a) signal aberrations caused when one or both electrodes momentarily lose connection with the body, a situation called “leads-off”, and, (b) when motion causes the captured signal contents to be distorted through the introduction of false peaks and crests, such as by contamination of the ECG signal with electromyographic signals from skeletal muscles. In either case, these signal artifacts need to be detected and rejected before heart rate and heart rate variability calculations are resumed. The SEP may also be subject to artifacts from various radio frequency devices, and even from operation of the host device. These may be detected and filtered or squelched/suppressed. For example, if the SEP is reliably interfered with by incoming calls to a smartphone host device, the smartphone host may communicate a signal to the SEP during receipt of such a call, to assist the SEP in interference processing. Appropriate error signals are sent by the SEP to the host device. The motion artifact detection technique implemented within the SEP (that is, on-board) is as follows. FIGS. 9A and 9B show the leads-off and motion/noise artifacts detected when SEP is on a human subject. As can be seen in FIG. 9A, the leads-off condition may lead to a saturation of the amplification circuitry and ADC. The signal may require a settling period after contact is reestablished, before the signal is valid again. As can be seen in FIG. 9B, the EMG or other artifact is superimposed on the ECG signal, and is detectable based on the signal morphology. The valid ECG signal returns shortly after the interference is mitigated, unless the input becomes saturated or overloaded, in which case a settling period would be required before data is again valid.

The ADT uses a technique called dynamic baseline matching which maintains the average value of the captured ECG signal within a moving window that advances with each ECG period. If the baseline calculated for the most recent period deviates significantly from the average calculated for the window, a leads-off condition is detected within the last period of measurement and the moving average is not updated with data from the current period and the window is simply moved forward by one period. If the leads-off condition is not detected, the moving average is updated, and the window is moved forward. By maintaining a dynamic window, natural variations in the ECG pattern caused by sweating and normal temporal changes caused by slow motion are allowed for.

When either motion/noise artifact or leads off is detected the MPT in software momentarily suspends data processing, and resets all thresholds, buffers, and baseline while it waits for a good ECG signal to resume processing. It recomputes the ECG baseline, and MPT goes through pre-processing again to reestablish the baseline. If noise, or leads-off signal lasts for over 2 seconds, it loops in pre-processing until there's a true R-peak detected. Extended periods of useful signal acquisition losses trigger the transmission of an error code to the host for notification/intervention.

The ADT monitors the baseline consistency, running at a frequency of 10 Hz in parallel with the MPT. After the pre-preprocessing phase, the MPT processes each sample in the ECG signal in two steps: First, it monitors for real-time slope change in the input ECG signal. For every transition of the signal's slope from positive to negative (implying a potential R-peak), followed by a transition from a negative slope to a positive slope (implying a potential S-peak) it records the peak as a potential R-peak in a Peak Buffer (PB) in memory. The PB, e.g., size of 4 bytes, can hold two values where one value is the true R-Peak, and the second value is the potential R-peak which is compared with the true R-peak to determine if it is a true R-peak. If the comparison returns “True” then the most recent true R-peak is stored in the buffer, and the R-peak preceding it is deleted from the buffer to make place for the next potential R-peak. The acquired signal for a single period is considered as a motion artifact and unacceptable if any one of the following conditions are true:
a. The detected R-peak's amplitude is not within ±ΔR of the last measured baseline from accepted signals. This threshold (ΔR) is also configurable. Any signal peak outside this range is considered as a motion artifact. For the implementation presented here, AR is 30% above/below the most recently estimated baseline average. It is to be noted that this threshold value is empirical and determined from an analysis of the archived signals in the Massachusetts Institute of Technology-Beth Israel Hospital Arrhythmia Database (MIT BIH Database, physionet.org/content/mitdb/1.0.0/) (as well as actual measurements on subjects).
b. The potential R-peak is a high-frequency signal peak, with inter-peak distances less than the highest expected heart rate (240 bpm), that is with inter-peak distances of less than 250 mS and peak amplitudes less than 10% of the running baseline average for accepted signals. These potential R-peaks are ignored as high frequency noise.
c. The potential R-peak implies a heartbeat rate outside an expected heartbeat range of 30 to 240 bpm (beats per minute), corresponding to R-to-R peak intervals of 2000 and 250 mS, respectively. The peaks meeting this criterion are considered as induced by motion artifacts. Again, this range of acceptable heartbeat rates is pre-configurable.

One implementation uses independent threads to detect the rejection conditions for R-peaks mentioned under (a) through (c) above. When acceptable signals are identified, the baseline is estimated by averaging consistent acceptable signals over the past two seconds without the accepted R-peak and the Q-peak that follows it. Leads-off conditions indicating signals that need to be also rejected are identified as follows: (i) The acquired signal's R-peak within a 2 second interval is over 30% of the value of the R-peak baseline of accepted signals or if the acquired signal has saturated at the highest possible acquired signal value, or (ii) The acquired signal's peak value over the 2-second interval is between the least possible signal value and less than 30% of the baseline. If the period of two potential peaks is greater than the period-threshold, then the MPT regards the input signal as an ECG R-peak and calculates the instantaneous heart rate. MPT maintains a global counter, which counts every sample processed. This counter helps to determine the period between consecutive R-peaks and helps to calculate the instantaneous heart rate. This technique has low complexity compared to other peak detection techniques such as the one described in (Pan et al. 1985) which are not practical to implement in an onboard processing system like SEP.

Once the initial data acquisition, ADT, and heartrate (e.g., R wave interval) dependent calculations are established, the determined valid QRS waves may then be analyzed to encode the relevant parameters, e.g., the 13 parameters identified with respect to FIG. 2 and Table I.

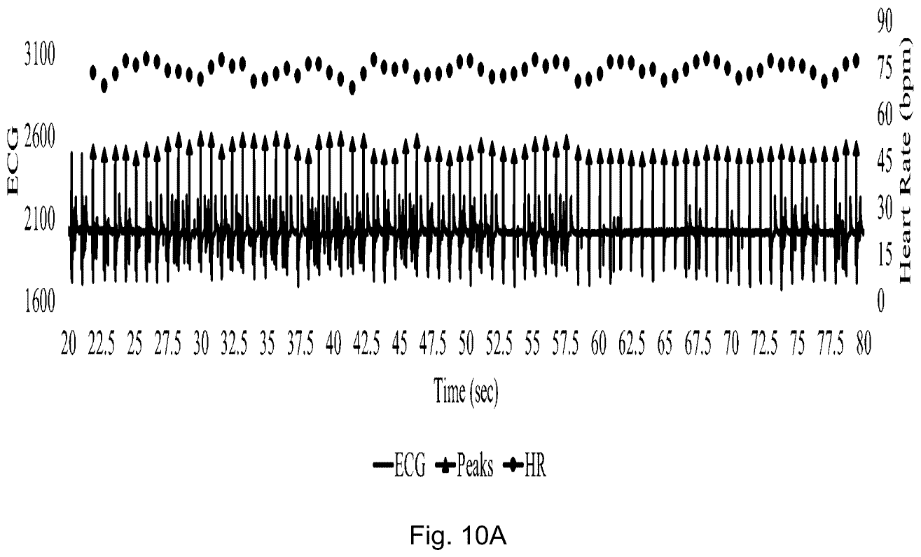

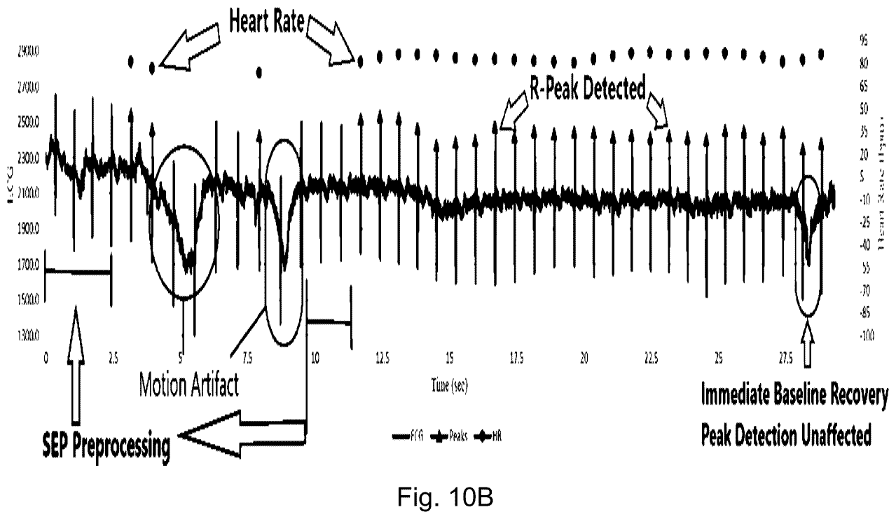

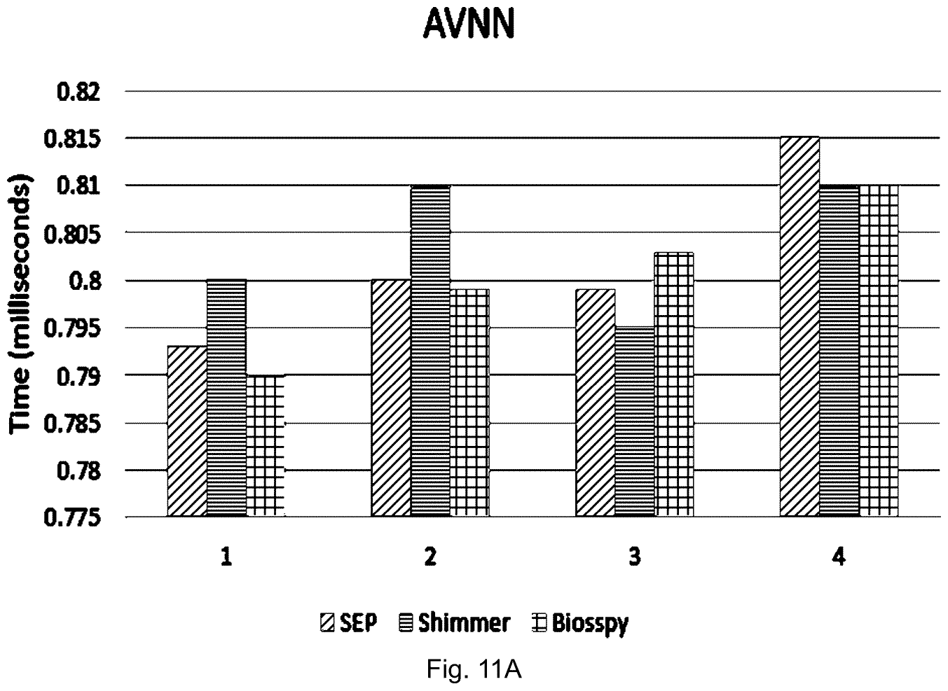

As shown in FIG. 10A, detected R-peaks, and instantaneous heart rate computed by SEP's MPT is shown as it was seen on the Bluetooth host. As shown in FIG. 10B, the MPT of the SEP detects all R-peaks, instantaneous heart rate, and the ADT detects motion artifacts in the ECG signal from a human subject. (Appropriate IRB protocols were in place.) For this experiment, the SEP continuously transmitted data to the host in real-time at a 100% duty cycle.

Another scenario where the transmissions are limited/restricted is when the ADT detects cither a motion/noise artifact, or a leads-off condition. In these cases, an alarm signal is sent to the connected Bluetooth host device so that the host/user is made aware of the situation. Also, all transmissions are paused until a good ECG signal is seen at the input of the SEP electrodes.

SEP uses wireless Bluetooth Low Energy (BLE) protocol (Gomez et al. 2012) to communicate over Bluetooth radio. The onboard software (TT and RT) uses BLE protocol stack API (Application Program Interface) for its communication purposes. The SEP's software application runs on top of a Generic Access Protocol (GAP) layer, and a Generic Attribute Profile (GATT) layer which are part of the Bluetooth Low Energy (BLE) protocol stack.

The SEP transmits, e.g., six different types of data over BLE to a connected Bluetooth host. To transmit ECG data sample (at a sampling rate of e.g., 400 samples per second), whose value ranges from 0-4095, is of the size 2 Bytes (16 bits), communicated over BLE in stream of 20 Byte (Payload size) packets. The transmission frequency depends on the duty cycle that the SEP is set to operate. Next, the instantaneous heart rate data is computed by the SEP in terms of beats-per-minute (bpm), ranging between 0-240 (or 255), of size 1 Byte, and a packet payload of 1 Byte. The transmission frequency depends on the SEP's duty cycle. An alarm signal is sent to the Bluetooth host by the SEP upon successful detection of noise/motion artifact in the incoming signal. This is a Boolean value and is transmitted only when its value is “True”. The alarm signal(s) may also be combined with the heart rate byte, for example as a 4-bit code, allowing a heart rate range of 240 and an alarm code range of 16. The heart rate variability parameters data computed by the SEP is a one-time transmission at the end of each 5-minute time interval. Three heart rate variability parameters are expressed in milliseconds and only pNN50 is expressed as a percentage. The packet payload size for heart rate variability parameters is 8 Bytes. The SEP also communicates its battery level to the Bluetooth host when there is a drop in its value by 1%. This message may also be piggybacked on another message. The 13 encoded parameters may be transmitted for each heartbeat, a group of 4-16 heartbeats, or other period, as a multibyte message, e.g., 26 bytes (2 bytes per each of the 13 parameters, plus heart rate data). The information may be packed more efficiently into a smaller number of bytes by sensitivity weighting the bits in each parameter, with bits thus allocated based on diagnostic or analytic significance range.

The technology reduces the total power consumption, thereby significantly improving endurance on a single coin battery. The SEP is powered, for example, by a CR2032 coin battery (3V, LiMnO₂, 235 mAH), which is a balanced tradeoff between the physical dimensions of the SEP and operation time on the battery. The most dominant source of energy consumption is in the Bluetooth communication with the host. SEP uses a variety of techniques to reduce its power consumption, ranging from transmission power adjustment to actually curbing transmission adaptively based on real-time acquisition and processing of ECG signals on-board.

One way to reduce this power is to reduce the transmission power (Wang et al. 2015) from the SEP from +5 dBm to −21 dBm. The required transmission power is a function of the proximity of the host to the SEP. At the lowest transmission power level (−21 dBm), the host proximity is limited to 1 to 3 meters, due to ambient conditions. SEP determines the desired transmit power at connection setup time by reading the value of Received Signal Strength Indicator (RSSI) obtained from the Bluetooth radio interface. The RSSI depends on the ambient conditions, distance between the SEP and the host and ambient signal interference.

Beyond the modulation of transmission power, additional power operating modes were developed onboard the SEP to operate at a duty cycle less than 100% to progressively reduce the operating power, extending the longevity of the battery life through adaptive transmission. The host device sends a command packet to the SEP to specify these operation modes. The duty cycle is expressed as a percentage of the time duration for which the SEP is active (sum of the duration of all active periods in which data acquisition, its on-board processing/analysis and continuous transmission takes place) divided by the total running time of the SEP (sum of all active and inactive periods). For the results related to power consumption, an 8 mV peak-to-peak synthetic ECG signal derived from the MIT-BIH database traces was used to drive SEP. This was done to produce consistent results in a controlled manner.

The Smart ECG Patch (SEP) is a self-contained ECG sensor that successfully performed all critical processing on-board. Specifically, it detected and corrected for signal artifacts, including motion artifacts, that enabled the accurate estimation of the heart rate, heart rate variability, and encoding of QRS waveform parameters of the wearer. The SEP also implements low power operating modes that prolong its operating time significantly by trading off between optimized functionality and overall power consumption.

The ability to perform onboard signal analysis is important for generating alarms reliably based on continuously-monitored wearer conditions and for realizing a very long battery life using adaptive transmissions of heart rate and heart rate variability signals.

The SEP may have dimensions of 1.25 inches by 2 inches (with the longer dimension related to the required electrode separation for ECG signal acquisition), and may use a small rechargeable battery, e.g., a flexible battery, instead of the primary lithium battery, and a 16-bit ADC and may employ more sophisticated onboard processing, and analysis of ECG signal, while realizing a long battery life. The rechargeable battery may be associated with an energy harvesting system. For example, the battery may be recharged by a photovoltaic cell, an inductive coil for RF energy harvesting, a magnet moving with respect to a coil to harvest mechanical energy, or other known types of energy harvesting systems.

Machine learning techniques may be used estimate the various rejection thresholds and rejection intervals to make them specific to subjects and specific to types of movements.

### QRS Complex Based ECG Signal Encoding

The QRS waveform parameter encoding is employed as follows. Using the encoded ECG signal received via a Bluetooth link, a deep learning neural network model executes on the host device to detect and classify arrhythmia in real time. The resulting system, called ACES (Arrhythmia Classification using Encoded ECG Signals), can be used for critical cardiac health monitoring and advanced real-time diagnostics. ACES employs a Bidirectional Long-Short Term Memory (BILSTM) based neural network model to detect and identify six distinct classes of arrhythmia with a high degree of accuracy. Each of these six classes corresponds to a morphologically unique arrhythmia. A separate class is used for normal ECG signals (“normal sinus rhythm”). Data from the MIT-BIH Arrhythmia dataset and human subjects were used in the evaluation of a prototype system, following appropriate IRB protocols for human subjects testing. The ECG signal encoding also saves considerable energy in transmitting data to the host device as only a small amount of encoded data is transmitted per ECG cycle instead of a full set of ECG signal samples.

After the QRS complex is detected, 13 parameters are extracted from it; these are as shown in FIG. 2, and as described in Table I. The parameter selection considers the fact that because of sampling by the analog-to-digital converter of SEP, the acquired ECG signal is not available as a continuous function of time and its peak locations and values are approximated, as time is quantized due to sampling, whereby a peak can occur between two consecutive sampling points. Consequently, from Table I, Parameters 2, 3, 6 and 7 are generally not an exact absolute value, but instead is a nearest digitized ECG sample to the absolute value, which is extracted to be part of the encoded ECG signal. The percentage figures for Parameters 6 and 7 were chosen empirically and nearby percentage values did not show any appreciable impact on the overall classification accuracy. For Parameters 8 through 13 in Table I, time is measured as the sample index (offset) from start of ECG cycle. These 13 parameters capture various nuances of the QRS complex and account for time quantization that some of the peaks sampled correspond to, or are close to, the actual peaks and their amplitudes. This enables the multiclass arrhythmia classifier to classify specific types of arrhythmias which have discernible changes in the morphological characteristics of QRS complex. The final and 14-th parameter used is the instantaneous heart rate (HR) as discussed above. By using 13 parameters derived from the QRS complex along with the instantaneous HR, ACES provides a dramatic edge over other techniques that detect and classify specific arrhythmias that differ in terms of intrinsic morphological characteristics of QRS complex.

The instantaneous HR is derived from the temporal distance between two consecutively detected R-peaks. This is an important parameter chosen for encoding as it provides average HR over time. For instance, when HR is 60 beats per minute (bpm) there is exactly one ECG pulse in every 400 digitized ECG samples, given that the ECG signal sampling period used for digitization used is 400 Hertz (Mittal et al., 2020). After detecting the QRS complex and encoding, 13 samples are stored in SEP's onboard memory for transmission to the host device. If SEP were to transmit a full ECG signal (without encoding), then for a 60-bpm normal sinus rhythm, and with 2 Bytes per sample, 800 Bytes of data has to be transmitted for one ECG waveform per second. In contrast, with encoding, SEP needs to transmit only 28 Bytes (14×2 Bytes), representing the QRS complex and the instantaneous HR, reducing the size of data transmitted per second by 96.5%. The reduction in the size of data to be transmitted continuously for real-time arrhythmia detection translates to power savings, as transmission power dominates the energy consumption total for SEP (Mittal et al., 2020). The duration needed to transmit 14 encoded parameters per ECG pulse's duration for a 60-bpm ECG signal is less than the time needed to acquire 400 samples of ECG. Consequently, SEP idles transmission in a low power mode by not transmitting any data during the remaining 386 (=400-14) sampling intervals, saving significant battery energy. During this idling period data acquisition and QRS complex detection continues to run on SEP, but these require substantially lower energy compared to data transmission.

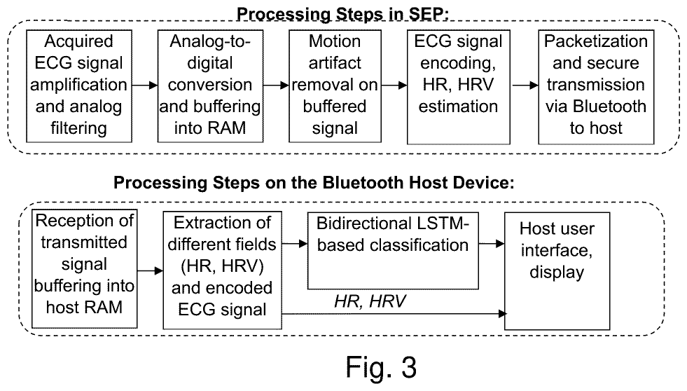

The top row of FIG. 3 depicts the main steps involved in acquiring signals on SEP (Mittal et al., 2020), the on-board analytics for reducing artifacts (Mittal et al., 2020), the buffering and proposed ECG signal encoding (above), followed by the transmission step. It is worth noting that complete, artifact-free ECG waveforms are acquired on board for encoding and preferably only the encoded signals are transmitted. The bottom row of FIG. 3 shows the steps performed on the Bluetooth host side device—typically a smartphone, smartwatch, tablet, or laptop/PC. On both SEP and the host device, the steps run continuously in cycles. For SEP, as shown later, the transmission step consumes the highest power, overshadowing the power consumption of all other steps.

### Arrhythmia Classification From Encoded ECG

ACES, the multi-class arrhythmia classifier presented here, classifies an incoming stream of encoded ECG signal from SEP into seven different classes. Classes 1 through 5 (Table 2) uniquely identify the dysfunction in the conduction system of the heart by detecting morphological changes in ECG patterns. All other types of arrhythmias are detected and classified as other/generic arrhythmia (Class 6 in Table 2) purely based on abnormalities in HR. The variations in morphological characteristics of arrhythmias that are classified into Class 6 are not limited to QRS complex. They span from variations in P-wave, QRS complex and ST variations, thus classifications of such arrhythmias are purely done by detecting variations in the instantaneous HR. The 7th class is a normal sinus rhythm (normal ECG), which represents normal sinus rhythm without any arrhythmia.

### A. Data Preparation and Feature Selection

ACES is trained using the MIT-BIH Arrhythmia database (Goldberger et al. 2000, Moody et al, 2001). This is an open-source database which contains ECG signal recordings from 47 subjects and it contains a mix of inpatient and outpatient ECG signals. ECG signal recordings are digitized at 360 Hz using a 11-bit resolution. This dataset is annotated to label true arrhythmia rhythms in ECG. The MIT-BIH dataset is first scanned to identify different annotations quantitatively, as shown in Table 2. The ECG signals corresponding to roughly 42 subjects are chosen for training ACES. To ensure a fair and balanced split between the training and validation sets during training, 20% of the samples from each type of arrhythmia class are part of the validation dataset. The remaining 80% samples of each class of arrhythmia are part of the training dataset. The remaining ECG data in the MIT database from 5 subjects are chosen to be part of the test dataset used for evaluating the final system (and are not used in training or validation). To complete the test data set, archived data from human subject tests acquired using SEP are added. Similarly, the total number of annotated normal sinus rhythms, or normal heartbeat is 75,052. These are also divided into training, validation, and test datasets similarly.

Arrhythmia, as defined previously, is an irregular heart rhythm. By this it means that arrhythmia primarily depends on the rate of change of instantaneous heart rate. However, monitoring just the heart rhythm is only a binary identification of arrhythmia in an ECG signal which sets it apart from the normal sinus rhythm. If we delve deeper into classification of different types of specific arrhythmias then we need to look closely into the intrinsic morphological characteristics of ECG waveform. In ACES' arrhythmia classifier, the six arrhythmias are uniquely identified by determining the changes in the characteristics of extracted 14 parameters from the QRS complex per ECG pulse of the ECG signal, which are a part of the encoded ECG transmission data from SEP. One hundred samples for each of the 14 features is the input to the ACES arrhythmia classifier.

### B. Multi-Class Classifier Model Architecture of ACES

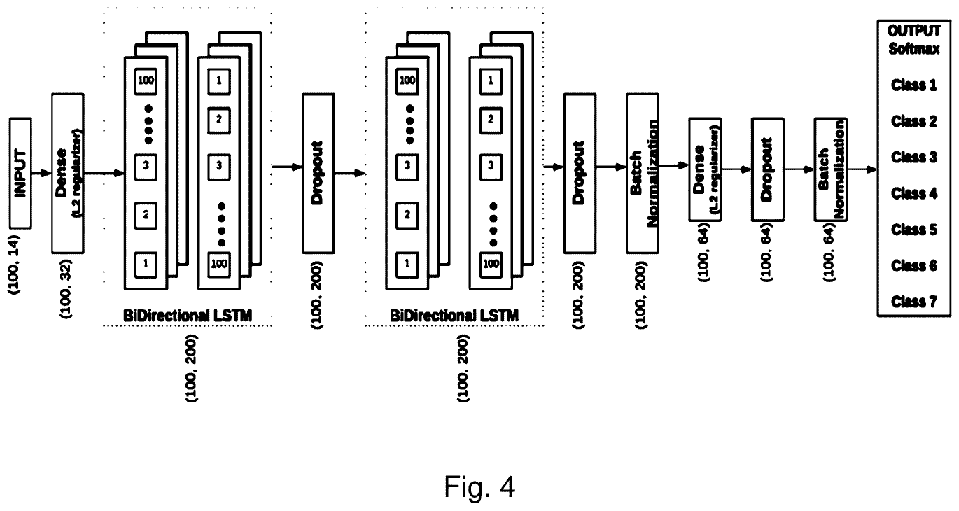

The ECG signal is a sequence of data which varies as a function of time wherein each sample has interdependence on the sample before it and a sample after itself. BILSTM (Shuster et al. 1997), a variant of recurrent neural networks Zihlmann et al. 2017), is efficient in deriving knowledge from time series data with respect to forward and backward directions in time. ACES uses BILSTM layers to learn a weighted relationship between samples in encoded ECG. A set of 14 parameters represent the encoded ECG pulse. ACES' classifier model is trained to learn the long-term time dependency across consecutive sets of these 14 encoded parameters with respect to time. ACES has an edge over a deep CNN based implementations because ECG features required for arrhythmia classification are already extracted. Therefore, a deep CNN is not required to extract features from input ECG data. FIG. 4 represents the model architecture of ACES. A 100-ECG-sample timestep in each batch is used as input to the model, which enables the model to handle both low HR (30 bpm) and high HR (220 bpm), based on the sampling frequency of SEP (400 Hz).

A SoftMax activation is used in the final output layer (FIG. 4), which always returns a probability distribution over the seven classes in this multiclass classification model. The SoftMax function ensures that negative inputs will be converted into nonnegative values because it uses an exponential function, which always returns a probability distribution over the seven classes in the proposed multiclass arrhythmia classification model. Thus, values will be in range of 0 to 1. As the denominator in each SoftMax computation is the same, the output values are proportional to each other. Overfitting is avoided by adding regularizing dropout layers. In each layer of the BILSTM, there are 100 LSTM cells in the forward direction LSTM layer and 100 LSTM cells in each backward direction LSTM layer. A cross-entropy loss function is used to adjust the weights during backpropagation in training. The Adam optimizer is used with default values for its beta1 and beta2 parameters. However, its epsilon parameter is set to 1e-08. The learning rate is set to 0.001. A total of 362,055 parameters are used in the model, of which 361,527 parameters are trainable. The BiLSTM is implemented using the Keras library with a TensorFlow backend and trained on a NVIDIA Geforce RTX 2070 GPU.

### C. Results

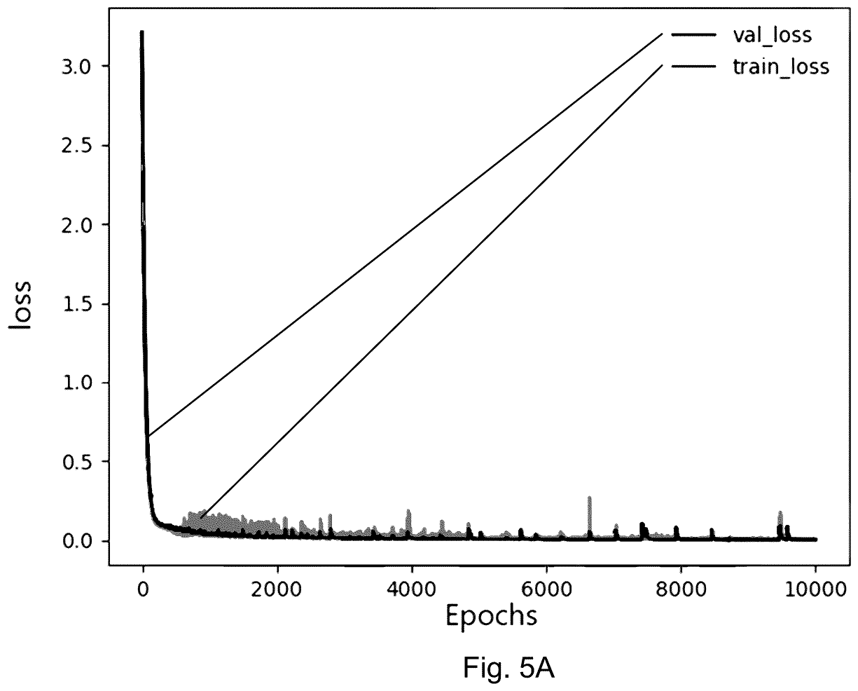

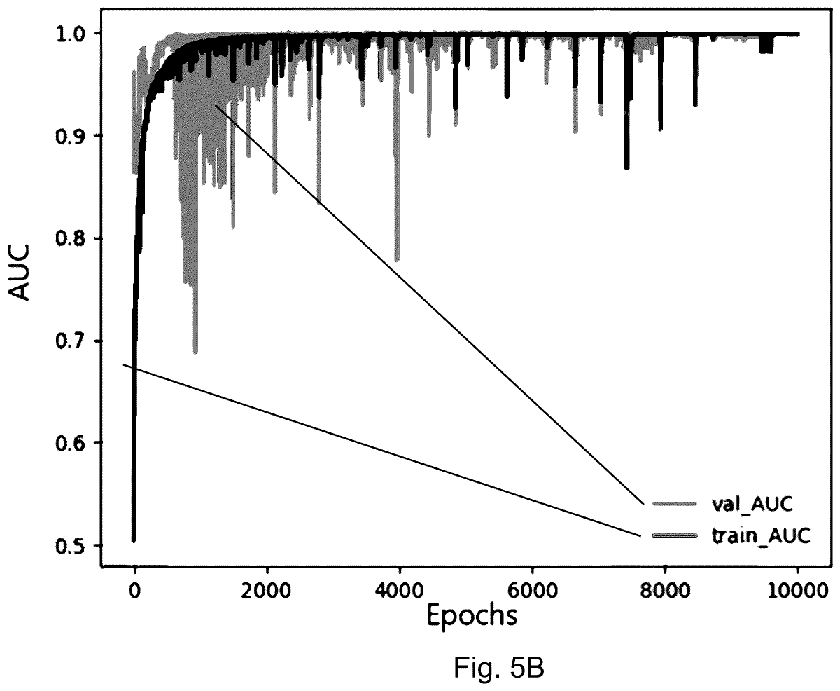

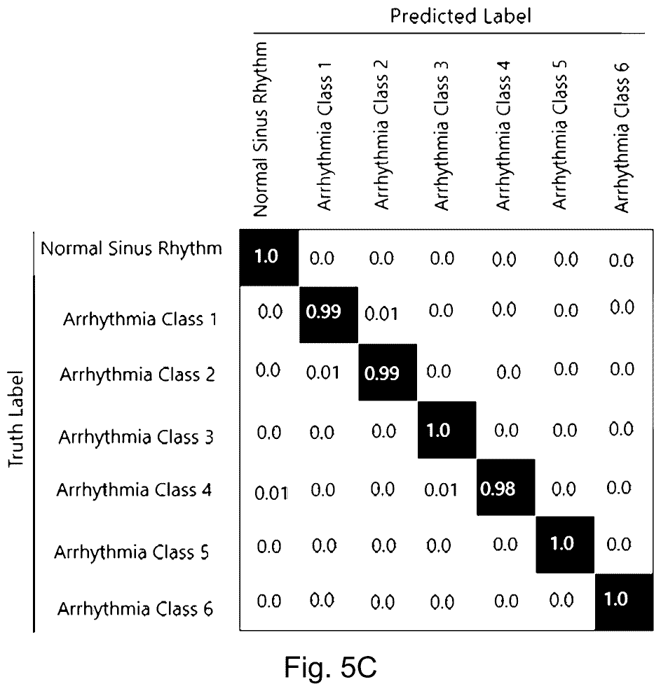

A 10-fold cross-validation is used to evaluate ACES' model performance on training, validation, and test datasets. After training, the model converges between 5,000 epochs and 10,000 epochs. So, 10,000 epochs proved to show promising results. The training loss of the model converges to 0.0152 and the validation loss converges to 0.0342, as shown in FIG. 5A. AUC (Area Under receiver operating characteristic Curve) is chosen as a metric to determine model's performance, as it provides a broader view of performance of the classifier by giving a rank correlation between predicted class and target class. Therefore, it gives a probability that a randomly chosen positive instance is ranked higher than randomly chosen negative instance, which gives the confidence measure of the model to classify arrhythmia into seven classes. This means that a higher value of AUC directly corresponds to a higher confidence of the model in classifying arrhythmia into seven classes. The AUC plot is shown in FIG. 5B, where the training AUC is found to be 0.9956 and the validation AUC reaches 0.9898 after 10,000 epochs. In addition, a portion of the test dataset is built using similar principles as the validation set, containing ECG data which is unseen during training and validation (and distinct from those used for training) containing data from all classes of arrhythmia. The test data set is completed by adding archived signals from human subjects acquired with SEP. ACES' model performance is evaluated over the test dataset where the predicted label is compared to the truth label for each class of the classifier.

A confusion matrix is used to evaluate the quality of the classifier, as shown in FIG. 5C. To clearly show which class is being misclassified and to handle unequal number of elements in each class of test dataset, the confusion matrix elements, ranging from 0 to 1, are normalized across all seven arrhythmia classes. The diagonal elements in the confusion matrix represents the rate of true positives detected, whereas the off-diagonal elements represent the rate of false positives detected in the test dataset. In FIG. 5C, all diagonal elements have a high value (greater than or equal to 0.98) that signifies a high true positive detection performance of ACES. Also, the off-diagonal elements in FIG. 5C have a value very close to, or equal to 0. This indicates that the rate of false positive detection in ACES is very low. Hence, the AUC of evaluation on test dataset is 0.9864, which is as good as the AUC observed during training of ACES.

### Battery Power Evaluation

The goal of the proposed ECG encoding technique is to save battery power on SEP while preserving the integrity of ECG signal, performing real time arrhythmia classification and ECG waveform reconstruction. Battery power can be evaluated by measuring the continuous current drawn by the sensor. This is achieved by placing a 1.5 Ohm resistor in series with the SEP's CR2032 battery. By continuously measuring the voltage drop across this resistor (starting from powering on to powering off SEP) the current drawn by SEP can be derived using Ohm's law. An Arduino Due (store.arduino.cc/products/arduino-due) board is used to continuously record the voltage drop across the resistor over time. The current drawn by the SEP during encoded ECG transmission is compared with the current drawn during a full ECG transmission.

ACES can classify arrhythmia continuously in real time. Real time operation corresponds to continuous (100% duty cycle) transmission of the encoded ECG signal between SEP and the connected Bluetooth host device. In (Mittal et al., 2020), significant power saving has been achieved for SEP without the use of any encoding by altering the transmission duty cycle based on various parameters and decisions made by SEP. However, no power saving was achieved during continuous transmission of full ECG signal by SEP in (Mittal et al., 2020). Using the ECG encoding technique of ACES, SEP can transmit fewer amount of data over a shorter interval per ECG pulse. (Recall that the encoded ECG has only 14 parameters per ECG pulse, which is significantly lower than 400 samples of a full ECG pulse)

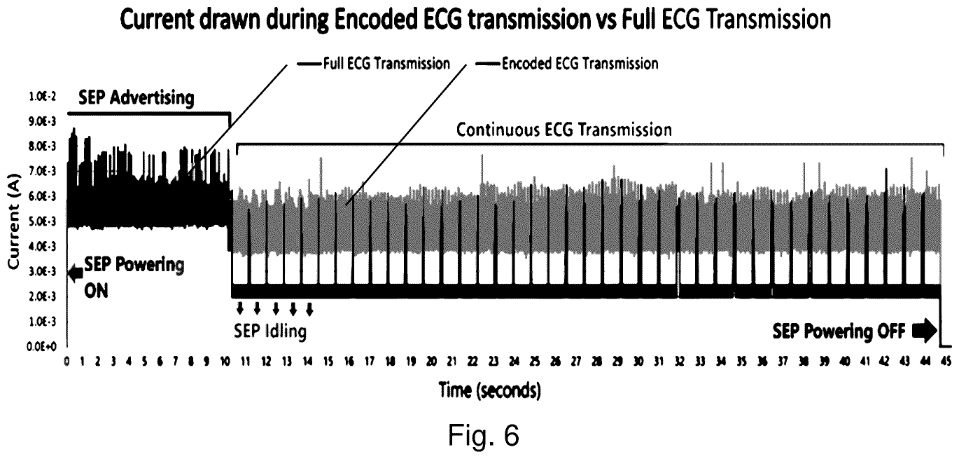

FIG. 6 shows the current drawn by SEP at different times during its operation when using ACES. Initially, when SEP is turned on, it starts advertising until it is connected to a Bluetooth host device. Advertising consumes most battery power, as seen in FIG. 6. After establishing a successful Bluetooth connection, continuous transmission of the encoded ECG signal is initiated by the SEP sensor with a lower current draw, shown in lower trace. The SEP transmits the encoded signals for a single ECG period and idles at a lower power thereafter. While idling, SEP is only acquiring ECG, running onboard algorithms to process and encode ECG, and maintaining an active Bluetooth connection with the host device. In contrast, continuous transmission of the ECG samples as they are acquired, consumes a higher power (shown in upper trace), leaving no opportunity for SEP to idle. The average peak current drawn by SEP during any transmission over its Bluetooth radio is about 6 mA. The total capacity of the CR2032 battery powering SEP is 210 mAh. Thus, if SEP were transmitting a full ECG signal continuously, in theory, the battery will last about 35 hours. On the other hand, it is observed that when SEP transmits an encoded ECG signal it uses only about 15% of the ECG cycle and idles for the rest 85% of the time. The average peak current drawn, estimated from the data collected for the plot of FIG. 6 is about 2.5 mA throughout the idling part of the ECG period and averages at 3.1 mA during the entire period. The lower average power consumption with encoded ECG transmission results in an operation time of 68 hours on a single CR2032 battery. Table 3 compares the SEP with and without the ECG signal encoding. The encoding technique almost doubles the operation time of SEP on a CR2032 coin battery from 35 hours to almost 68 hours for continuous operations (100% duty cycle), even though the arrhythmia classification feature is added. With duty cycling, that is interleaved periods of continuous operations and quiescent times, the operation time is prolonged even more significantly, as seen in Table 3.

ACES is a technique that encodes each ECG pulse with 13 features derived from the QRS complex together with the instantaneous heart rate (14^{th} parameter) to permit the detection and classification of arrhythmia based solely on the encoded representation. The encoding is implemented in real time on a small wearable ECG patch and the encoded representation is sent to a Bluetooth-connected host device for arrhythmia classification and detection on the host device in real time. Demonstrations using clinical data shows that ACES performs the arrhythmia classification with a high degree of accuracy. The encoding technique of ACES dramatically reduces the amount of data that need to be transmitted from the wearable sensor and prolongs the battery life for the sensor quite significantly, as data transmission needs are cut down commensurately. From a practical standpoint, ECG signal encoding, as presented, gives a very quick and reasonably accurate way of detecting arrhythmia—enough to trigger the use of the complete ECG signal transmission from the sensor for more accurate clinical diagnosis using the complete, unencoded ECG waveforms when needed. At present ACES is being improved further to classify arrhythmia at a finer granularity and even higher accuracy using encoding for the entire ECG pulse.

### Heart Rate Variability Calculation

The definition and classification of heart rate variability parameters is given in (Shaffer et al. 2017). Onboard the SEP, four short-term time-domain heart rate variability parameters are estimated, and buffered on a limited flash memory of 128 KB. Average of all NN intervals (AVNN), Standard Deviation of all NN intervals (SDNN), Square root of the mean of squares of differences between adjacent NN intervals (RMSSD), Percentage of differences between adjacent NN intervals that are greater than 50 mS (pNN50) are the four heart rate variability parameters estimated onboard SEP. Instantaneous heart rate is buffered in memory every 5 minutes. Maximum size of this buffer is 1200 Bytes, and this buffer can thus hold 1200 instantaneous heart rate values. The buffer is reset and cleared at the end of 5-minute interval after the heart rate variability parameters are estimated.

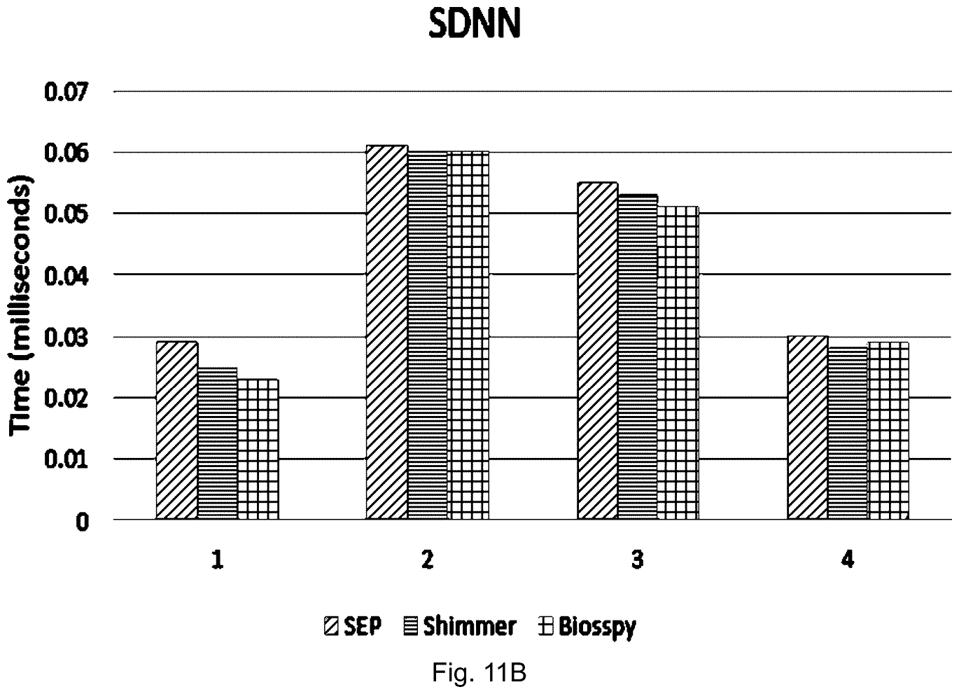

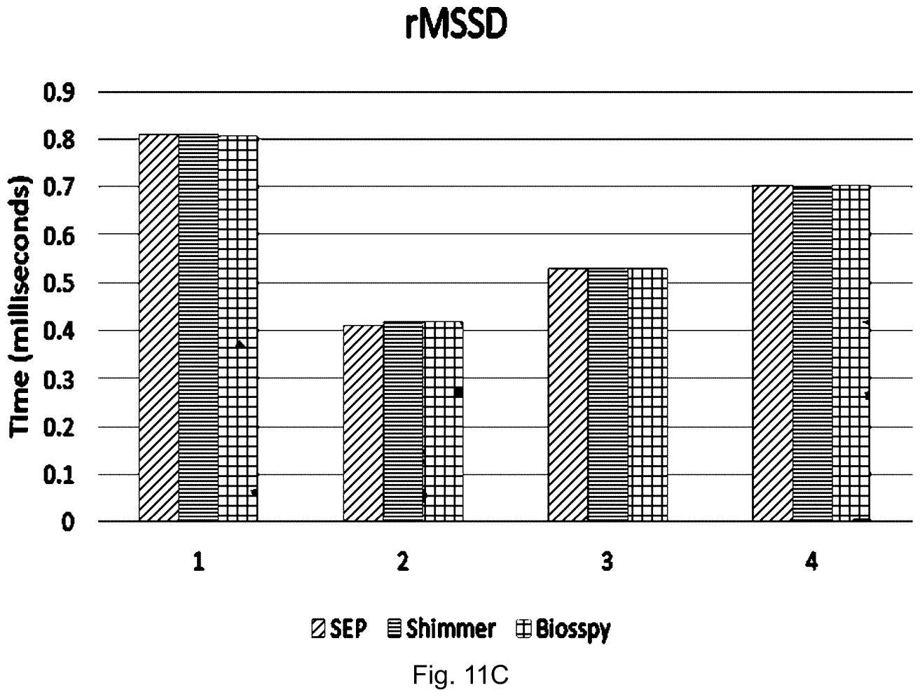

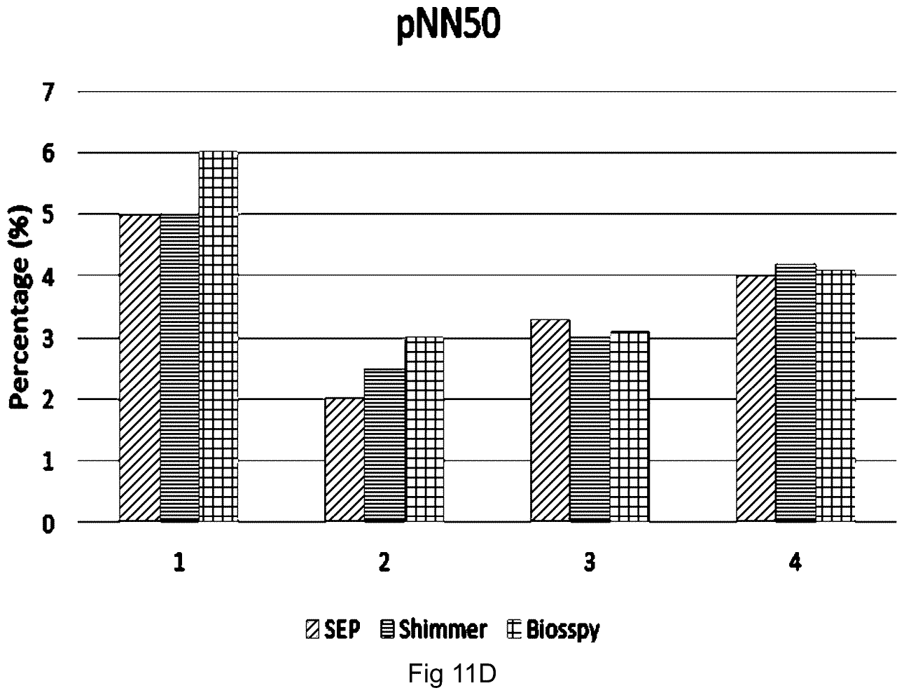

The heart rate variability parameters estimated by the SEP are compared with the heart rate variability parameters estimated by Shimmer3, and BioSPPy (Carreiras et al. 2018) on the same ECG signal. To do this, the SEP transmits the instantaneous heart rate at 100% duty cycle to the connected Bluetooth Host. All the incoming instantaneous heart rate is buffered on the host, it runs two threads, one runs the Shimmer heart rate variability computation, and other runs BioSPPy heart rate variability computation. The ECG signals used to estimate the heart rate variability parameters are sourced from the MIT-BIH database, and from human subjects. The comparison of heart rate variability parameters from all three is shown in a FIGS. 11A-11D, for Average of all NN intervals (FIG. 11A AVNN), Standard Deviation of all NN intervals (FIG. 11B SDNN), Square root of the mean of squares of differences between adjacent NN intervals (FIG. 11C RMSSD), and Percentage of differences between adjacent NN intervals that are greater than 50 mS (FIG. 11D pNN50). It shows that the onboard SEP's computation of heart rate variability parameters is as accurate as heart rate variability computation done on a Bluetooth host using Shimmer and BioSPPy.

To ensure a secure connection and mitigate eavesdroppers, brute force key attacks, and other security breaches, the SEP's MCU implements Elliptic Curve Cryptographic (ECC) algorithm stored in its Read-Only Memory (ROM). (Rajashrec 2021, Kumar et al. 2019, Banerjee et al. 2018, Gyami et al. 2019, Sowjanya 2020). The use of ECC combined with a hardware-based true random number generator ensures that the key generated is resistant against the security attacks. In addition, the SEP uses a randomly generated Bluetooth device address in its advertising mode. The random address is derived from a cryptographic function and it changes periodically, typically every 15 minutes. Only after a secure connection is setup with a trusted host device, is the SEP's real Bluetooth device address disclosed along with an Identity Resolving Key (IRK). This way, untrusted devices, who do not have an IRK, will have no way of tracking advertisements from the SEP, thereby the SEP's real Bluetooth device address cannot be resolved for malicious purposes. Further, all packets in-between the SEP and its hosts are encrypted using AES-128 using 16 Byte (128 bit) keys to protect the sensitive data and wearer's privacy.

Four short-term time domain HRV parameters (Shaffer et al. 2017) are computed on board of SEP at 5-minute time intervals. The SEP has a limited in-System flash memory of 128 KB, thus, short term HRV is estimated onboard. Long term HRV (over a 24-hour period) are computed on Bluetooth Host side. The SEP acquired and processed ECG signal from the MIT-100 certified ECG signal database and human subjects. BioSPPy (Carreiras et al. 2015), an ECG toolbox developed in python, was used to do a comparative study of SEP's HRV computation accuracy. In FIGS. 11A-11D, results from the comparative study show that SEP's onboard processing of HRV is as accurate as the BioSPPy HRV computation done on a PC.

### Power Consumption Modes

In decreasing order of operating power, the SEP power (and operation) modes are as follows:

### A. Continuous Transmission Mode (CONT)

The SEP captures, processes and sends the ECG signal, and optionally the heart rate and heart rate variability, and encoded QRS parameters to the host continuously. The heart rate and heart rate variability data are e.g., interspersed with ECG data every minute. The QRS parameters may be sent individually or in groups, or with the heart rate data. The heart rate variability data represents the results collected in the past 5 minutes, while heart rate is instantaneous heart rate computed whenever a R-peak is detected. The battery life is minimum and is determined by the specified transmission power level.

### B. Simple Duty Cycling (SD) Power Mode

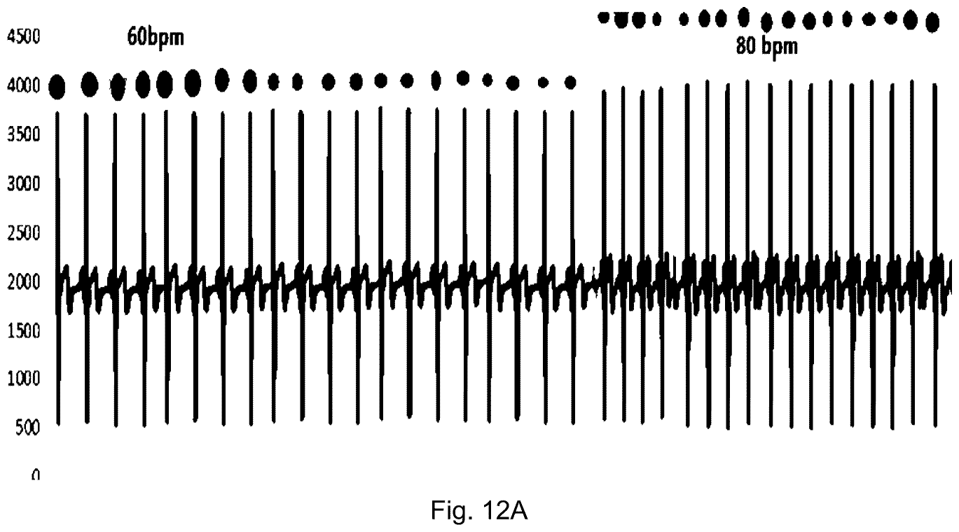

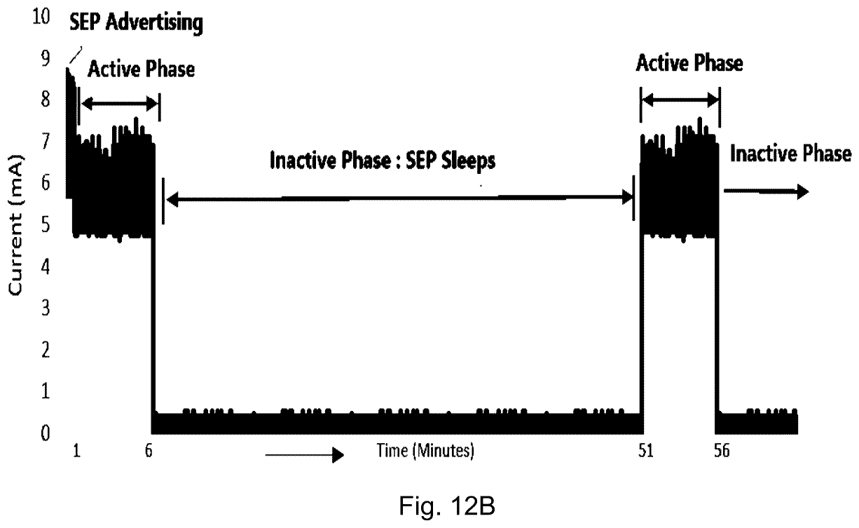

The SEP uses host-dictated duty cycle management technique to ensure that overall average current draw is kept lower than in the CONT transmission mode. In this mode, the SEP operates continuously for 5 minutes (independent of the duty cycle specified) in the CONT mode and then goes to sleep in the lowest power mode possible for time specified by the duty cycle. For instance, when the duty cycle specified is 40% (that is operate for 40% of a cycle and sleep for 60% of the cycle time), the SEP operates in the CONT mode for 5 minutes and then goes to sleep for 7.5 minutes. During the sleep phase, the processing of signals from ECG electrodes, motion artifact/noise removal, peak detection, heart rate/heart rate variability estimation, QRS signal encoding, and transmission to the host are all suspended. On wakeup via a timer interrupt from the sleep phase, SEP notifies its presence to the host and reconnects to it and then resumes operation in the CONT mode for the next active phase for 5 minutes. ECG, heart rate transmission, and the Current drawn by SEP (without QRS encoding) is shown in FIG. 12B. The power savings in this mode comes from keeping the SEP inactive during the quiescent period. However, nothing is done within the active phase to save power in this operation mode.

### C. Adaptive Duty Cycling with Continuous Heart Rate Transmission and Triggered ECG Transmission (AD-CHR+TE) Power Mode

In a mode triggered by HR variation (A-ECG), the transmission of the ECG data is done adaptively during the active phase of the duty cycle. The instantaneous heart rate payload is only one Byte long and needs to be transmitted only when a new R-peak is detected by the SEP's MPT. The QRS waveform encoding requires further data transmission capacity, e.g., 26 bytes per waveform plus the heartrate information. The instantaneous heart rate and QRS waveform encoded data transmission, compared to the ECG data transmission, saves transmission energy because of its small payload size and lower transmission rate. This mode is similar to the DC mode, but: (a) only the instantaneous heart rate and optionally the encoded QRS waveform is transmitted during the active phase, followed by the heart rate variability at the end of the active phase if the heart rate stays within a threshold; (b) heart rate, encoded QRS waveform parameters, heart rate variability, and the ECG waveforms are sent to the host during the active part only when the heart rate falls outside the threshold. The threshold is 10% of the estimated instantaneous heart rate on either side of the running average heart rate.

In the A-ECG mode, the heart rate variability parameters are transmitted at the end of the 5-minute active period just as in the previous power mode. Note that in this mode, the heart rate variability parameters are computed onboard the SEP from all ECG data collected during the past 5-minute active period even when it's not transmitted to the host. Compared to the SD mode, if the heart rate stays within the specified range, the power savings are realized by not transmitting the ECG waveform during the active phase.

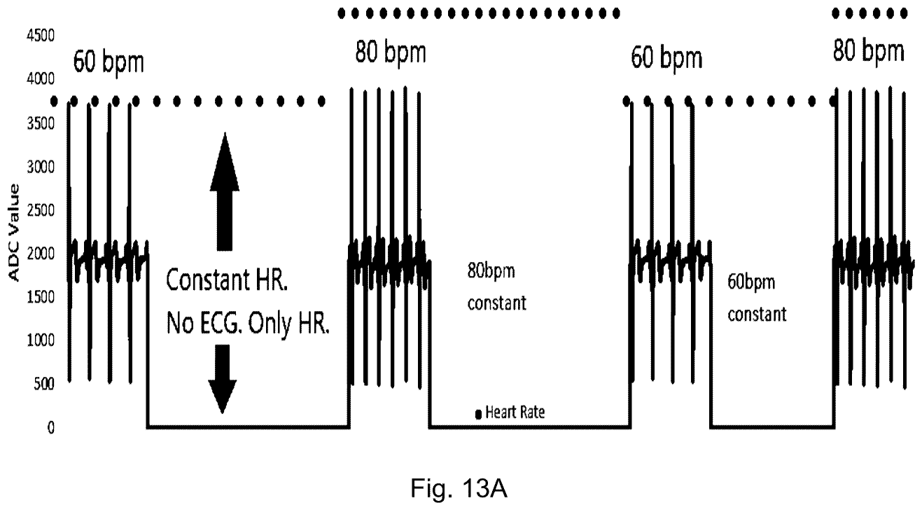

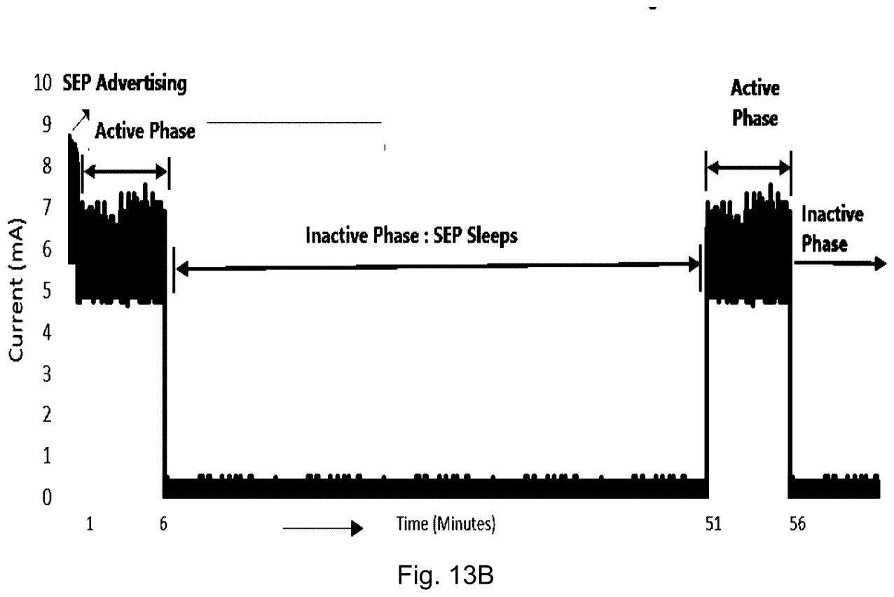

As shown in FIG. 13A, ECG and heart rate transmission are shown as displayed on a Bluetooth host, and current drawn by SEP (without QRS parameter encoding) in this phase is plotted in FIG. 13B. The power savings are exploited in cases when the human subject is at rest, or performing a physical activity at almost constant rate, for example, walking at almost constant pace.

### D. Adaptive Duty Cycling with Triggered Heart Rate and ECG Transmission (AD-TR+TE) Power Mode

This mode extends power savings further beyond the previous mode (AD-CHR+TE) by not sending any heart rate, encoded QRS parameters, heart rate variability or ECG signal to the host during the active phase if the heart rate stays within the heart rate threshold (and optionally of the QRS parameters are within a normal range). In lieu of these signals, a keep-alive signal is sent to the host to inform that the SEP is operating properly even though it has not sent the heart rate, heart rate variability and ECG signals. The heart rate threshold is the same as described for the previous mode. This power mode of operation is the most aggressive, power-efficient, and conservative mode of the SEP software. It realizes the highest power savings among the operation modes described. All true detections of either a motion/noise artifact or leads off condition are handled as described previously. Characteristics of inactive period in this operating mode are the same as the one described in previous power mode.

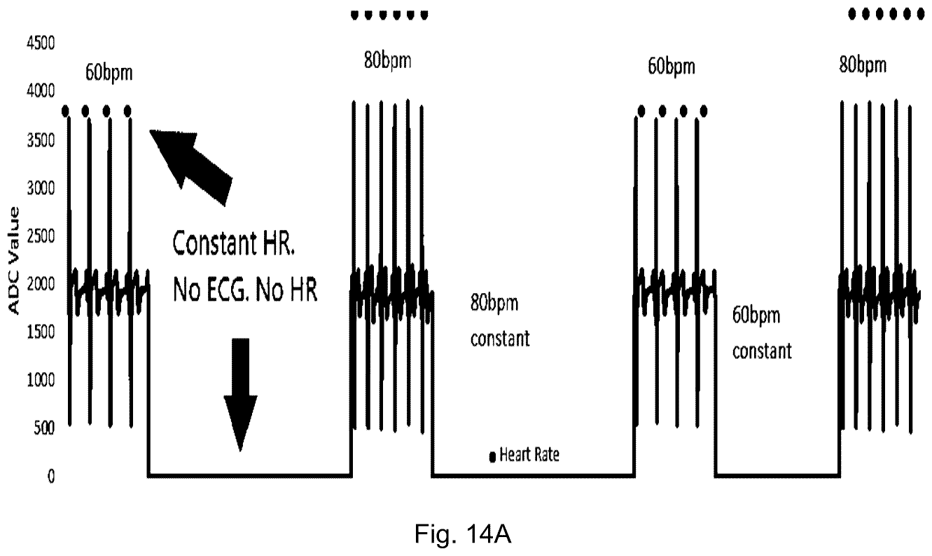

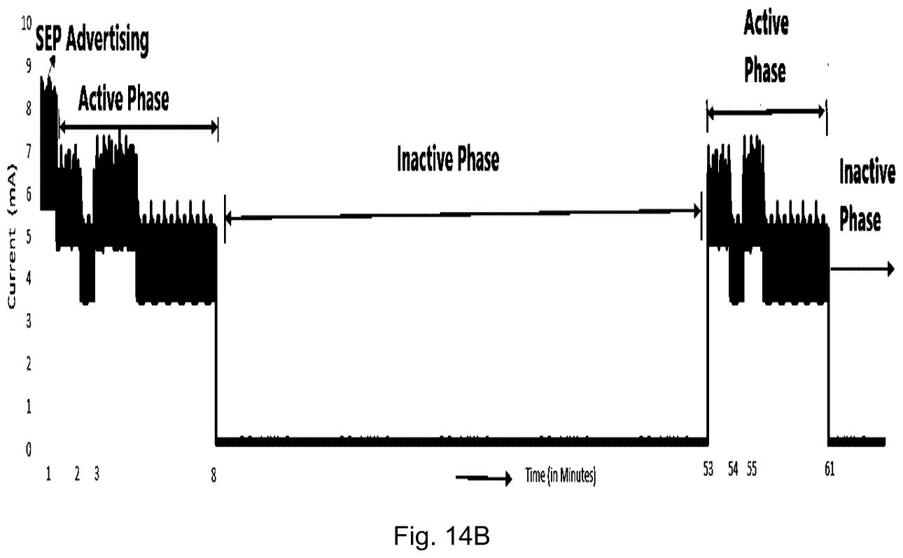

In FIG. 14A, ECG and heart rate transmission received by the Bluetooth host is shown.

In FIG. 14B, current drawn by SEP in this power mode (without QRS waveform parameter encoding) is plotted.

The SEP's power consumption (without encoding of QRS waveform parameters) in the normal mode and the various power-conserving modes, was measured using a National Instruments (NI) USB-6259 Data Acquisition Module with NI LabVIEW Software Application based on the voltage drop across a small resistor (1.5 Ohms) in series with the SEP's battery. The battery life was measured as the time from the moment a SEP was turned on (with a fresh battery) to the time the battery voltage dropped below 1.8 V (the operating range for TI CC2640 is 1.8 V-3.8 V). The battery life is determined at three operating duty cycles: 10%, 50%, and 100%.

Table 4 shows the average battery current during active phase when the SEP (without encoding of QRS waveform parameters) transmits at a specific duty cycle. It also shows the average current consumption during inactive mode of the duty cycle in each of the three duty cycle modes. The right-most column in this table shows the battery life, in hours, noted at three duty cycles. A set of three SEPs were used for the battery life measurements; All had one CR2032 coin cell battery each. Each set of SEP was operated at the three duty cycles, with each run using a new coin battery. The battery life for these three duty cycle modes was determined as the average battery life seen with each of the three SEPs by allowing the SEP to drain the battery to its fullest until SEP disconnects the BLE connection to the host (at a battery voltage of 1.8 Volts) and turns off. The number of operating hours were noted for these duty cycle operation modes. With the SEP software's duty cycle management for power saving, peak detection and BLE radio on the battery lasts on the average at least 27.8 hours at 100% duty cycle, 54.9 hours at 50% duty cycle, and 249.6 hours at 10% duty cycle.

The claims of this document define certain combinations and subcombinations of elements, features and steps or operations, which are regarded as novel and non-obvious. Additional claims for other such combinations and subcombinations may be presented in this or a related document. These claims are intended to encompass within their scope all changes and modifications that are within the true spirit and scope of the subject matter described herein.

TABLE 1
PARAMETERS EXTRACTED FROM QRS COMPLEX

| | Parameter Description (Temporal) distance is |
|---|---|
| Parameter from | difference in indices of samples used within |
| FIG. 2 | an ECG period, along the time axis (FIG. 2) |
| 1 | Sampled highest signal amplitude, used as the R-peak |
| 2 | Nearest sample at 80% of used R-peak (1) along QR |
| 3 | Nearest sample at 60% of used R-peak (1) along QR |
| 4 | Sampled amplitude of Q-peak |
| 5 | Sample amplitude of S-peak |
| 6 | Nearest sample at 60% of used R-peak (1) along RS |
| 7 | Nearest sample at 80% of used R-peak (1) along RS |
| 8 | Distance between samples used for (1) and (2) |
| 9 | Distance between samples used for (1) and (3) |
| 10 | Distance between samples used for Q- and R-peaks |
| 11 | Distance between samples used for R-and S-peaks |
| 12 | Distance between samples for Parameter 6 and R-peak |
| 13 | Distance between samples for Parameter 7 and R-peak |

TABLE 2
QUANTITATIVE CLASSIFICATION OF ANNOTATIONS
IN ARRHYTHMIA CLASSIFIER

| Class | Type of Arrhythmia | Number of Annotations |
|---|---|---|
| 1 | Left Bundle Branch Block | 8075 |
| 2 | Right Bundle Branch Block | 7259 |
| 3 | Premature Ventricular Contraction | 7130 |
| 4 | Paced Beat | 7028 |
| 5 | Atrial Premature Beat | 2546 |
| 6 | Other/Generic | 5557 |

TABLE 3
SEP BATTEY LIFE

| | Full ECG Transmission | | Encoded ECG Transmission | |
|---|---|---|---|---|
| Transmission | Average | Battery | Average | Battery |
| Duty Cycle | Current | Life | Current | Life |
| (%) | (mA) | (Hours) | (mA) | (Hours) |
| 100 | 6.0 | 35.0 | 3.1 | 67.74 |
| 50 | 3.07 | 68.4 | 1.62 | 129.63 |
| 10 | 0.67 | 313.43 | 0.31 | 552.15 |

### 100% Duty Cycle=Continuous Real Time Transmission/Operation

TABLE 4
BATTERY LIFE ESTIMATION FOR
SEP AT DIFFERENT DUTY CYCLES

| | Active Period | | Inactive Period | | Total | Battery CR2032 | |
|---|---|---|---|---|---|---|---|
| Duty | Avg | | Avg | | Average | | Battery |
| Cycle | Current | Time | Current | Time | Current | Capacity | Life |
| (%) | (mA) | (s) | (mA) | (s) | (mA) | (mAh) | (hours) |
| 100 | 5.54 | 3600 | 0.07 | 0 | 5.54 | 220 | 27.8 |
| 50 | 5.54 | 1800 | 0.07 | 1800 | 2.80 | 220 | 55.0 |
| 10 | 5.54 | 360 | 0.07 | 3240 | 0.62 | 220 | 249.6 |

References (Each reference cited herein is expressly incorporated herein by reference for all purposes)

“CALM-M Class I Medical Device for Hospitals and Home Care.” CALM. www.calm-health.com/calm-healthcare/(CALM-M).

“Heart Arrhythmia.” Mayo Clinic, Mayo Foundation for Medical Education and Research, 9 Aug. 2020, www.mayoclinic.org/diseases-conditions/heart-arrhythmia/symptoms-causes/syc-20350668 (Mayo, 2020).

Agyeman, Michael Opoku, Andres Felipe Guerrero, and Quoc-Tuan Vien. “A review of classification techniques for arrhythmia patterns using convolutional neural networks and Internet of Things (IOT) devices.” IEEE Access (2022).

Akrivopoulos, Orestis, et al. “Design and Evaluation of a Person-Centric Heart Monitoring System over Fog Computing Infrastructure.” Proceedings of the First International Workshop on Human-centered Sensing, Networking, and Systems. ACM, 2017.

Alfaras, Miquel, Miguel C. Soriano, and Silvia Ortín. “A fast machine learning model for ECG-based heartbeat classification and arrhythmia detection.” Frontiers in Physics 7 (2019): 103.

Ali, Hassan, Ben Ernest Villaneouva, and Raziq Yaqub. “Design and Implementation of a Low Cost Wireless Ambulatory ECG Monitoring System for Deployment in Rural Communities.” International Journal of Online and Biomedical Engineering (iJOE) 15, no. 15 (2019): 57-79.

Anliker, Urs, et al. “AMON: a wearable multiparameter medical monitoring and alert system.” IEEE Transactions on information technology in Biomedicine 8.4 (2004): 415-427.

Baba, Elhoussaine, Abdelillah Jilbab, and Ahmed Hammouch. “A health remote monitoring application based on wireless body area networks.” Intelligent Systems and Computer Vision (ISCV), 2018 International Conference on. IEEE, 2018.

Baheti, Ashutosh, Anshul Jain, Yamini Goyal, and Amit Neogi. “Bluetooth Controlled Life Savior System.”

Bai, Baodan, Yufang Zhao, Xinrong Chen, Yingmin Chen, and Zhangyuan Luo. “A smart portable ECG monitoring system with high precision and low power consumption.” Journal of Intelligent & Fuzzy Systems Preprint: 1-11.

Baig, Mirza Mansoor, Hamid Gholamhosseini, and Martin J. Connolly. “A comprehensive survey of wearable and wireless ECG monitoring systems for older adults.” Medical & biological engineering & computing 51.5 (2013): 485-495.

Banerjee, Soumi, and Anita Patil. “ECC based encryption algorithm for lightweight cryptography.” In International conference on intelligent systems design and applications, pp. 600-609. Springer, Cham, 2018.

Benade, S. A., and U. L. Bombale. “FINGER TOUCH BASED ECG MONITORING.” IJRET: International Journal of Research in Engineering and Technology eISSN: 2319-1163| pISSN: 2321-7308, Volume: 05 Issue: 07| July-2016, ijret.esatjournals.org

Bhamra, Hansraj Singh. “Micro-power circuits and systems for wireless sensor nodes and implantable medical devices.” (2016).:Purdue University, Ph.D. Dissertation.

Carreiras, C., et al. “BioSPPy: Biosignal processing in Python.” Accessed on 3.28 (2015): 2018.

Chang, Robert Chen-Hao, Hung-Lieh Chen, Chih-Hung Lin, and Kuang-Hao Lin. “Design of a low-complexity real-time arrhythmia detection system.” Journal of Signal Processing Systems 90, no. 1 (2018): 145-156.

Chatzigiannakis, Ioannis, and Christos Tselios. “Internet of Everything.” In Intelligent Computing for Interactive System Design: Statistics, Digital Signal Processing, and Machine Learning in Practice, pp. 21-56. 2021.

Chen, Shuwen, Jiaming Qi, Shicheng Fan, Zheng Qiao, Joo Chuan Yeo, and Chwee Teck Lim. “Flexible Wearable Sensors for Cardiovascular Health Monitoring.” Advanced Healthcare Materials (2021): 2100116.

Chiang, Cheng-Yi, Hong-Hui Chen, Tung-Chien Chen, Chien-Sheng Liu, Yu-Jie Huang, Shey-Shi Lu, Chii-Wann Lin, and Liang-Gee Chen. “Analysis and design of on-sensor ECG processors for realtime detection of VF, VT, and PVC.” In 2010 IEEE Workshop On Signal Processing Systems, pp. 42-45. IEEE, 2010.

Chlaihawi, Amer Abdulmahdi, et al. “Development of printed and flexible dry ECG electrodes.” Sensing and Bio-Sensing Research (2018).

Chowdhury, Muhammad E H, et al. “Wearable RealTime Heart Attack Detection and Warning System to Reduce Car Accidents in Qatar.” Qatar Foundation Annual Research Conference Proceedings. Vol. 2018. No. 2. Qatar: HBKU Press, 2018.

Christoe, Michael J., Jialuo Han, and Kourosh Kalantar-Zadeh. “Telecommunications and data processing in flexible electronic systems.” Advanced Materials Technologies 5, no. 1 (2020): 1900733.

Chuo, Yindar, Marcin Marzencki, Benny Hung, Camille Jaggernauth, Kouhyar Tavakolian, Philip Lin, and Bozena Kaminska. “Mechanically flexible wireless multisensor platform for human physical activity and vitals monitoring.” IEEE transactions on biomedical circuits and systems 4, no. 5 (2010): 281-294.

Clark, Nicholar, Edward Sandor, Calvin Walden, In Soo Ahn, and Yufeng Lu. “A wearable ECG monitoring system for real-time arrhythmia detection.” In 2018 IEEE 61st International Midwest Symposium on Circuits and Systems (MWSCAS), pp. 787-790. IEEE, 2018.

Cosoli, Gloria, Susanna Spinsante, Francesco Scardulla, Leonardo D'Acquisto, and Lorenzo Scalise. “Wireless ECG and cardiac monitoring systems: state of the art, available commercial devices and useful electronic components.” Measurement (2021): 109243.

Coulter, Simon, et al. “Low power IoT platform for vital signs monitoring.” Signals and Systems Conference (ISSC), 2017 28th Irish. IEEE, 2017.

Dai, Ming, Xueliang Xiao, Xin Chen, Haoming Lin, Wanqing Wu, and Siping Chen. “A low-power and miniaturized electrocardiograph data collection system with smart textile electrodes for monitoring of cardiac function.” Australasian physical & engineering sciences in medicine 39, no. 4 (2016): 1029-1040.

De Capua, Claudio, Antonella Meduri, and Rosario Morello. “A smart ECG measurement system based on web-service-oriented architecture for telemedicine applications.” IEEE Transactions on Instrumentation and Measurement 59.10 (2010): 2530-2538.

Dey, P. S., and N. Kayalvizhi, “ECG System as Smartphone Peripheral,” 2019 9th International Conference on Advances in Computing and Communication (ICACC), Kochi, India, 2019, pp. 27-30, doi: 10.1109/ICACC48162.2019.8986187.

Farahabadi, Amin, et al. “Detection of QRS complex in electrocardiogram signal based on a combination of hilbert transform, wavelet transform and adaptive thresholding.” Biomedical and Health Informatics (BHI), 2012 IEEE-EMBS Int'l Conf. on. IEEE, 2012.

Faust, Oliver, Edward J. Ciaccio, and U. Rajendra Acharya. “A review of atrial fibrillation detection methods as a service.” International journal of environmental research and public health 17, no. 9 (2020): 3093.

Fensli, Rune, Einar Gunnarson, and Torstein Gundersen. “A wearable ECG-recording system for continuous arrhythmia monitoring in a wireless tele-home-care situation.” Computer-Based Medical Systems, 2005. Proceedings. 18th IEEE Symposium on. IEEE, 2005.

Fulford-Jones, Thaddeus R F, Gu-Yeon Wei, and Matt Welsh. “A portable, low-power, wireless two-lead EKG system.” In The 26th Annual International Conference of the IEEE Engineering in Medicine and Biology Society, vol. 1, pp. 2141-2144. IEEE, 2004.

Goldberger, Ary L., et al. “PhysioBank, PhysioToolkit, and PhysioNet: components of a new research resource for complex physiologic signals.” circulation 101.23 (2000): e215-e220.

Gomez, C., Oller, J., & Paradells, J. (2012). Overview and evaluation of bluetooth low energy: An emerging low-power wireless technology. Sensors, 12(9), 11734-11753.

Gong, Zhun, and Yaru Ding. “Design and Implementation of Wearable Dynamic Electrocardiograph Real-time Monitoring Terminal.” IEEE Access (2019).

Gotlibovych, Igor, Stuart Crawford, Dileep Goyal, Jiaqi Liu, Yaniv Kerem, David Benaron, Defne Yilmaz, Gregory Marcus, and Yihan Li. “End-to-end deep learning from raw sensor data: Atrial fibrillation detection using wearables.” arXiv preprint arXiv: 1807.10707 (2018).

Gupta, Varun, and Monika Mittal. “Arrhythmia detection in ECG signal using fractional wavelet transform with principal component analysis.” Journal of The Institution of Engineers (India): Series B 101, no. 5 (2020a): 451-461.

Gupta, Varun, Monika Mittal, and Vikas Mittal. “Chaos theory: an emerging tool for arrhythmia detection.” Sensing and Imaging 21, no. 1 (2020b): 1-22.

Gusev, Marjan, and Ana Guseva. “State-of-the-art of cloud solutions based on ECG sensors.” Smart Technologies, IEEE EUROCON 2017-17th International Conference on. IEEE, 2017.

Gyamfi, Eric, James Adu Ansere, and Lina Xu. “ECC based lightweight cybersecurity solution for IoT networks utilising multi-access mobile edge computing.” In 2019 Fourth International Conference on Fog and Mobile Edge Computing (FMEC), pp. 149-154. IEEE, 2019.

Hernandez-Silveira, M., S. S. Ang, T. Mehta, and B. Wangand A. Burdett. “Implementation And Evaluation Of A Physical Activity And Energy Expenditure Algorithm In A Sensium™-Based Body-Worn Device.” (2012). DOI: 10.5220/0003786902190223, In Proceedings oftheInternationalConferenceonBiomedicalElectronicsandDevices (BIODEVICES-2012), pages219-223, ISBN: 978-989-8425-91-1

Hirsch, Gerald, Søren H. Jensen, Erik S. Poulsen, and Sadasivan Puthusserypady. “Atrial fibrillation detection using heart rate variability and atrial activity: A hybrid approach.” Expert Systems with Applications 169 (2021): 114452.

Hou, Zhongjie, et al. “A Real-Time QRS Detection Method Based on Phase Portraits and Box-Scoring Calculation.” IEEE Sensors Journal 18.9 (2018): 3694-3702.

Hsiao, Chun-Chieh, Ren-Guey Lee, Sheng-Chung Tien, Yen-Yi Feng, and Shih-Feng Huang. “Early clinical prognosis for high-risk chest pain patients using smart textiles.” Biomedical Engineering: Applications, Basis and Communications 27, no. 06 (2015): 1550057.

Huang, Hui, Shiyan Hu, and Ye Sun. “Energy-efficient ECG compression in wearable body sensor network by leveraging empirical mode decomposition.” Biomedical & Health Informatics (BHI), 2018 IEEE EMBS International Conference on. IEEE, 2018.

Huanhuan, Meng, and Zhang Yue. “Classification of electrocardiogram signals with deep belief networks.” 2014 IEEE 17th International Conference on Computational Science and Engineering. IEEE, 2014.

Hui, Yi, Zhendong Yin, Mingyang Wu, and Dasen Li. “Wearable devices acquired ECG signals detection method using 1D convolutional neural network.” In 2021 15th International Symposium on Medical Information and Communication Technology (ISMICT), pp. 81-85. IEEE, 2021.

Ingolfsson, Thorir Mar, Xiaying Wang, Michael Hersche, Alessio Burrello, Lukas Cavigelli, and Luca Benini. “Ecg-tcn: Wearable cardiac arrhythmia detection with a temporal convolutional network.” In 2021 IEEE 3rd International Conference on Artificial Intelligence Circuits and Systems (AICAS), pp. 1-4. IEEE, 2021.

Islam, SM Riazul, et al. “The internet of things for health care: a comprehensive survey.” IEEE Access 3 (2015): 678-708.

Izumi, Shintaro, Hiroshi Kawaguchi, Masahiko Yoshimoto, and Yoshikazu Fujimori. “Normally-off technologies for healthcare appliance.” In 2014 19th Asia and South Pacific Design Automation Conference (ASP-DAC), pp. 17-20. IEEE, 2014.

Izumi, Shintaro, Ken Yamashita, Masanao Nakano, Hiroshi Kawaguchi, Hiromitsu Kimura, Kyoji Marumoto, Takaaki Fuchikami et al. “A Wearable Healthcare System With a 13.7 μA Noise Tolerant ECG Processor.” IEEE transactions on biomedical circuits and systems 9, no. 5 (2014): 733-742.

Jain, Anshul, Yamini Goyal, and Ajit Patel. “ECG Analysis System with Event Detection based on Daubechies Wavelets.” International Journal of Advanced Computer Research (ISSN (print): 2249-7277 ISSN (online): 2277-7970) 3 (2012).

Jang, Do-Hun, and SeongHwan Cho. “A 43.4 μW photoplethysmogram-based heart-rate sensor using heart-beat-locked loop.” Solid-State Circuits Conference-(ISSCC), 2018 IEEE International. IEEE, 2018.

Jani, Abhishek B., Ravi Bagree, and Anil K. Roy. “Design of a low-power, low-cost ECG & EMG sensor for wearable biometric and medical application.” SENSORS, 2017 IEEE, 2017.

Jha, Pankaj Kumar, and Asudeb Dutta. “Process Aware Analog-Centric Single Lead Ecg Acquisition And Classification Cmos Frontend.” PhD diss., Indian institute of technology Hyderabad, 2018.

Jin, Yanrui, Chengjin Qin, Jinlei Liu, Ke Lin, Haotian Shi, Yixiang Huang, and Chengliang Liu. “A novel domain adaptive residual network for automatic atrial fibrillation detection.” Knowledge-Based Systems 203 (2020): 106122.

Jin-ling, Zhang, et al. “An ECG 7-lead monitoring system designing based on lower-power.” Complex Medical Engineering (CME), 2013 ICME International Conference on. IEEE, 2013.

Jovin, I., Maslakovic, M., Maslakovic, M., Johnson, D., Jovin, I., Maslakovic, M., . . . Jovin, I. (2019, April 28). Keep tabs on your heart: Wearables that come with an ECG sensor. Retrieved from gadgetsandwearables.com/2018/09/22/ecg-sensor/

Kalaskar Radha B., and Bharati Harsoor. “An End-to-End Point of Cardiovascular Body Sensor Network with Cloud Service.” Bharati, An End-to-End Point of Cardiovascular Body Sensor Network with Cloud Service (May 17, 2019) (2019).

Kallas, Maya, et al. “Multi-class SVM classification combined with kernel PCA feature extraction of ECG signals.” 2012 19th International Conference on Telecommunications (ICT). IEEE, 2012.

Kartsch, V., G. Tagliavini, M. Guermandi, S. Benatti, D. Rossi and L. Benini, “BioWolf: A Sub-10-mW 8-Channel Advanced Brain-Computer Interface Platform With a Nine-Core Processor and BLE Connectivity,” in IEEE Transactions on Biomedical Circuits and Systems, vol. 13, no. 5, pp. 893-906, October 2019, doi: 10.1109/TBCAS.2019.2927551.

Kartsch, Victor, Fiorenzo Artoni, Simone Benatti, Silvestro Micera, and Luca Benini. “Using Low-Power, Low-Cost IoT Processors in Clinical Biosignal Research: an In-depth Feasibility Check.” In 2020 42nd Annual International Conference of the IEEE Engineering in Medicine & Biology Society (EMBC), pp. 4008-4011. IEEE, 2020.

Khalaf, Abdelbaset, and Rishaad Abdoola. “Wireless body sensor network and ECG Android application for eHealth.” Advances in Biomedical Engineering (ICABME), 2017 Fourth International Conference on. IEEE, 2017.

Khan, Yasser, et al. “Flexible Hybrid electronics: direct interfacing of soft and hard electronics for wearable health monitoring.” Advanced Functional Materials 26.47 (2016): 8764-8775.

Khandoker, Ahsan H., and Brian A. Walker. “Designing a Low-Cost ECG Sensor and Monitor: Practical Considerations and Measures.” Healthcare Sensor Networks: Challenges Toward Practical Implementation (2016): 339.

Kim, Hyejung, et al. “ECG signal compression and classification algorithm with quad level vector for ECG holter system.” IEEE Transactions on Information Technology in Biomedicine 14.1 (2009): 93-100.

Kim, Junho, Hycok Kim, Dongwook Kim, Hun-Jun Park, Kiwon Ban, Seungyoung Ahn, and Sung-Min Park. “A wireless power transfer based implantable ECG monitoring device.” Energies 13, no. 4 (2020): 905.

Kim, Sangkyu, Sangil Chon, Jin-Kook Kim, Joomin Kim, Yeongjoon Gil, and Sunghoon Jung. “Lightweight Convolutional Neural Network for Real-Time Arrhythmia Classification on Low-Power Wearable Electrocardiograph.” Annual International Conference of the IEEE Engineering in Medicine & Biology Society (EMBC), pp. 1915-1918. IEEE, 2022.

Kiranyaz, Serkan, et al. “Convolutional Neural Networks for patient-specific ECG classification.” 2015 37th Annual International Conference of the IEEE Engineering in Medicine and Biology Society (EMBC). IEEE, 2015 (Kiranyaz 2015b).

Kiranyaz, Serkan, Turker Ince, and Moncef Gabbouj. “Real-time patient-specific ECG classification by 1-D convolutional neural networks.” IEEE Transactions on Biomedical Engineering 63.3 (2015): 664-675. (Kirbanyaz 2015a)

Krachunov, Sammy, et al. “Energy efficient heart rate sensing using a painted electrode ECG wearable.” Global Internet of Things Summit (GloTS), 2017. IEEE, 2017.

Kumar, Devender, and Harmanpreet Singh Grover. “A secure authentication protocol for wearable devices environment using ECC.” Journal of Information Security and Applications 47 (2019): 8-15.

Kurl, Sudhir, et al. “Duration of QRS complex in resting electrocardiogram is a predictor of sudden cardiac death in men.” Circulation 125.21 (2012): 2588-2594.

Labati, Ruggero Donida, et al. “Deep-ECG: convolutional neural networks for ECG biometric recognition.” Pattern Recognition Letters 126 (2019): 78-85.

Lec, Kyong Ho, and Naveen Verma. “A low-power processor with configurable embedded machine-learning accelerators for high-order and adaptive analysis of medical-sensor signals.” IEEE Journal of Solid-State Circuits 48, no. 7 (2013): 1625-1637.

Lec, Seulki, Long Yan, Tachwan Roh, Sunjoo Hong, and Hoi-Jun Yoo. “A 75 μW real-time scalable network controller and a 25 μW E×G sensor IC for compact sleep-monitoring applications.” In 2011 IEEE International Solid-State Circuits Conference, pp. 36-38. IEEE, 2011.

Liang, Jifu, Shixiong Li, Ali Nikoofard, and Soumyajit Mandal. “A low-power receiver for simultaneous electrocardiogram and respiration rate detection.” In 2016 IEEE international symposium on circuits and systems (ISCAS), pp. 2455-2458. IEEE, 2016.

Lin, Qi, Weitao Xu, Guohao Lan, Yesheng Cui, Hong Jia, Wen Hu, Mahbub Hassan, and Aruna Seneviratne. “KEHKey: Kinetic Energy Harvester-based Authentication and Key Generation for Body Area Network.” Proceedings of the ACM on Interactive, Mobile, Wearable and Ubiquitous Technologies 4, no. 1 (2020): 1-26.

Lin, Qiuyang, Shuang Song, Iván D. Castro, Hui Jiang, Mario Konijnenburg, Roland van Wegberg, Dwaipayan Biswas et al. “Wearable Multiple Modality Bio-Signal Recording and Processing on Chip: A Review.” IEEE Sensors Journal 21, no. 2 (2020): 1108-1123.

Liu, Zengding, Bin Zhou, Zhiming Jiang, Xi Chen, Ye Li, Min Tang, and Fen Miao. “Multiclass Arrhythmia Detection and Classification From Photoplethysmography Signals Using a Deep Convolutional Neural Network.” Journal of the American Heart Association 11, no. 7 (2022): e023555.

Lobodzinski, S. Suave, and Michael M. Laks. “New devices for very long-term ECG monitoring.” Cardiology journal 19.2 (2012): 210-214.

Long, Yan, Yongli Chen, Deyong Xiao, Zheng Li, Tianpeng Hou, and Zhiwei Zhang. “Research on a Bluetooth Low Energy Warning Method.” In Journal of Physics: Conference Series, vol. 1631, no. 1, p. 012162. IOP Publishing, 2020.

Lou, Dongdong, Xianxiang Chen, Zhan Zhao, Yundong Xuan, Zhihong Xu, Huan Jin, Xingzu Guo, and Zhen Fang. “A wireless health monitoring system based on android operating system.” Ieri Procedia 4 (2013): 208-215.

Luz, Eduardo José da S., et al. “ECG-based heartbeat classification for arrhythmia detection: A survey.” Computer methods and programs in biomedicine 127 (2016): 144-164.

Majumder, S., L. Chen, O. Marinov, C. Chen, T. Mondal and M. J. Deen, “Noncontact Wearable Wireless ECG Systems for Long-Term Monitoring,” in IEEE Reviews in Biomedical Engineering, vol. 11, pp. 306-321, 2018, doi: 10.1109/RBME.2018.2840336.

Marsili, Italo Agustin, Luca Biasiolli, Michela Masè, Alberto Adami, Alberto Oliver Andrighetti, Flavia Ravelli, and Giandomenico Nollo. “Implementation and validation of real-time algorithms for atrial fibrillation detection on a wearable ECG device.” Computers in biology and medicine 116 (2020): 103540.

Meng, Lu, Kang Ge, Yang Song, Dongming Yang, and Zihuai Lin. “Long-term wearable electrocardiogram signal monitoring and analysis based on convolutional neural network.” IEEE Transactions on Instrumentation and Measurement 70 (2021): 1-11.

Meziane, N., J. G. Webster, Mokhtar Attari, and A. J. Nimunkar. “Dry electrodes for electrocardiography.” Physiological measurement 34, no. 9 (2013): R47.

Miao, Fen, et al. “A wearable context-aware ECG monitoring system integrated with built-in kinematic sensors of the smartphone.” Sensors 15.5 (2015): 11465-11484.

Mittal, Sandeep S., et al. “Low-Power Discreetly-Wearable Smart ECG Patch with On-Board Analytics.” 2020 IEEE International Symposium on Medical Measurements and Applications (MeMeA), pp. 1-6.

Moody G B, Mark R G. The impact of the MIT-BIH Arrhythmia Database. IEEE Eng in Med and Biol 20(3):45-50 (May-June 2001). (PMID: 11446209)

Mukhopadhyay, S. K., S. Mitra, and M. Mitra. “An ECG signal compression technique using ASCII character encoding.” Measurement 45.6 (2012): 1651-1660.

Naranjo-Hernández, David, Laura M. Roa, Javier Reina-Tosina, Gerardo Barbarov-Rostan, and Omar Galdamez-Cruz. “Smart device for the determination of heart rate variability in real time.” Journal of Sensors 2017 (2017).

Nemati, Ebrahim, M. Jamal Deen, and Tapas Mondal. “A wireless wearable ECG sensor for long-term applications.” IEEE Communications Magazine 50.1 (2012).

Noor, Safwat Mostafa. “Low energy computation methods for implantable cardiac pacemaker workloads.” PhD diss., The University of Texas at San Antonio, 2016.

Oweis, R. J., & A. Barhoum (2007) PIC microcontroller-based RF wireless ECG monitoring system, Journal of Medical Engineering & Technology, 31:6, 410-418, DOI: 10.1080/03091900600703560

Ozkan, Haydar, Orhan Ozhan, Yasemin Karadana, Muhammed Gulcu, Samet Macit, and Fasahath Husain. “A Portable Wearable Tele-ECG Monitoring System.” IEEE Transactions on Instrumentation and Measurement 69, no. 1 (2019): 173-182.

Pan, Jiapu, and Willis J. Tompkins. “A real-time QRS detection algorithm.” IEEE transactions on biomedical engineering 3 (1985): 230-236.

Park, Chulsung, et al. “An ultra-wearable, wireless, low power ECG monitoring system.” Biomedical Circuits and Systems Conference, 2006. BioCAS 2006. IEEE, 2006.

Pereira, Tania, Nate Tran, Kais Gadhoumi, Michele M. Pelter, Duc H. Do, Randall J. Lec, Rene Colorado, Karl Meisel, and Xiao Hu. “Photoplethysmography based atrial fibrillation detection: a review.” NPJ digital medicine 3, no. 1 (2020): 1-12.

Petmezas, Georgios, Kostas Haris, Leandros Stefanopoulos, Vassilis Kilintzis, Andreas Tzavelis, John A. Rogers, Aggelos K. Katsaggelos, and Nicos Maglaveras. “Automated atrial fibrillation detection using a hybrid CNN-LSTM network on imbalanced ECG datasets.” Biomedical Signal Processing and Control 63 (2021): 102194.

Pholpoke, Bhirawich, Techapon Songthawornpong, and Woradorn Wattanapanitch. “A Micropower Motion Artifact Estimator for Input Dynamic Range Reduction in Wearable ECG Acquisition Systems.” IEEE transactions on biomedical circuits and systems 13, no. 5 (2019): 1021-1035.

Poliks, Mark, et al. “A wearable flexible hybrid electronics ECG monitor.” Electronic Components and Technology Conference (ECTC), 2016 IEEE 66th. IEEE, 2016.

Ra, Ho-Kyeong, et al. “I am a Smart watch, Smart Enough to Know the Accuracy of My Own Heart Rate Sensor.” Proceedings of the 18th International Workshop on Mobile Computing Systems and Applications. ACM, 2017.

Rajashree, R. “A Comprehensive Survey on Lightweight Asymmetric Key Cryptographic algorithm for Resource Constrained Devices.” SPAST Abstracts 1, no. 01 (2021).

Rajashree, Radha BK. “Real-Time Ambulatory Monitoring System.” (2017). International Research Journal of Engineering and Technology (IRJET) e-ISSN: 2395-0056, Volume: 04 Issue: 05| May-2017

Ramesh, Jayroop, Zahra Solatidehkordi, Raafat Aburukba, and Assim Sagahyroon. “Atrial fibrillation classification with smart wearables using short-term heart rate variability and deep convolutional neural networks.” Sensors 21, no. 21 (2021): 7233.

Randazzo, V., J. Ferretti and E. Pasero, “ECG WATCH: a real time wireless wearable ECG,” 2019 IEEE International Symposium on Medical Measurements and Applications (MeMeA), Istanbul, Turkey, 2019, pp. 1-6. doi: 10.1109/MeMeA.2019.8802210

Randazzo, Vincenzo, Jacopo Ferretti, and Eros Pasero. “A wearable smart device to monitor multiple vital parameters-VITAL ECG.” Electronics 9, no. 2 (2020): 300.

Sankman, Robert L., Ian A. Young, Johanna M. Swan, and Marko Radosavljevic. “Electronic bio monitoring patch.” U.S. patent application Ser. No. 13/719,360, filed Jun. 19, 2014.

Schuster, Mike, and Kuldip K. Paliwal. “Bidirectional recurrent neural networks.” IEEE Transactions on Signal Processing 45.11 (1997): 2673-2681.

Sen-Gupta, Ellora, Donald E. Wright, James W. Caccese, John A. Wright Jr, Elise Jortberg, Viprali Bhatkar, Melissa Ceruolo et al. “A Pivotal Study to Validate the Performance of a Novel Wearable Sensor and System for Biometric Monitoring in Clinical and Remote Environments.” Digital Biomarkers 3, no. 1 (2019): 1-13.

Shaffer, Fred, and J. P. Ginsberg. “An overview of heart rate variability metrics and norms.” Frontiers in public health 5 (2017): 258.

Shao, Minggang, Zhuhuang Zhou, Guangyu Bin, Yanping Bai, and Shuicai Wu. “A wearable electrocardiogram telemonitoring system for atrial fibrillation detection.” Sensors 20, no. 3 (2020): 606.

Shimmer3 ECG Unit. (n.d.). Retrieved from www.shimmersensing.com/products/shimmer3-ecg-sensor (Shimmer3)

Shin, Seung-chul, et al. “Two electrode based healthcare device for continuously monitoring ECG and BIA signals.” Biomedical & Health Informatics (BHI), 2018 IEEE EMBS International Conference on. IEEE, 2018.

Singh, Mandeep, Gurmohan Singh, Jaspal Singh, and Yadwinder Kumar. “Design and Validation of Wearable Smartphone Based Wireless Cardiac Activity Monitoring Sensor.” Wireless Personal Communications (2021): 1-17.

Soh, Ping Jack, et al. “Wearable wireless health monitoring: Current developments, challenges, and future trends.” IEEE Microwave Magazine 16.4 (2015): 55-70.

Sowjanya, K., Mou Dasgupta, and Sangram Ray. “An elliptic curve cryptography based enhanced anonymous authentication protocol for wearable health monitoring systems.” International Journal of Information Security 19, no. 1 (2020): 129-146.

Spanò, Elisa, Stefano Di Pascoli, and Giuseppe Iannaccone. “Low-power wearable ECG monitoring system for multiple-patient remote monitoring.” IEEE Sensors Journal 16.13 (2016): 5452-5462.

Spinsante, Susanna, Sara Porfiri and Lorenzo Scalise. “Accuracy of Heart Rate Measurements by a Smartwatch in Low Intensity Activities.” 2019 IEEE International Symposium on Medical Measurements and Applications (MeMeA) (2019): 1-6.

Sufi, Fahim, and Ibrahim Khalil. “Diagnosis of cardiovascular abnormalities from compressed ECG: a data mining-based approach.” IEEE transactions on information technology in biomedicine 15.1 (2010): 33-39.

Sylvester, S. S., et al. “Miniaturized and Wearable Electrocardiogram (ECG) Device with Wireless Transmission.” Journal of Telecommunication, Electronic and Computer Engineering (JTEC) 9.3-9 (2017): 15-19.

Tan, Puchuan, Yang Zou, Yubo Fan, and Zhou Li. “Self-powered wearable electronics.” Wearable Technologies 1 (2020).

Tantawi, Manal M., et al. “A wavelet feature extraction method for electrocardiogram (ECG)-based biometric recognition.” Signal, Image and Video Processing 9.6 (2015): 1271-1280.

Toral, Víctor, Antonio García, Francisco J. Romero, Diego P. Morales, Encarnación Castillo, Luis Parrilla, Francisco M. Gómez-Campos, Antonio Morillas, and Alejandro Sánchez. “Wearable system for biosignal acquisition and monitoring based on reconfigurable technologies.” Sensors 19, no. 7 (2019): 1590.

Uchiyama, Akira, Shunsuke Saruwatari, Takuya Mackawa, Kazuya Ohara, and Teruo Higashino. “Context Recognition by Wireless Sensing: A Comprehensive Survey.” Journal of Information Processing 29 (2021): 46-57.

Valchinov, Emil, Athanasios Antoniou, Konstantinos Rotas, and Nicolas Pallikarakis. “Wearable ECG system for health and sports monitoring.” In 2014 4th International Conference on Wireless Mobile Communication and Healthcare-Transforming Healthcare Through Innovations in Mobile and Wireless Technologies (MOBIHEALTH), pp. 63-66. IEEE, 2014.

Vizitiu, Anamaria, Cosmin-loan Nita, Radu Miron Toev, Tudor Suditu, Constantin Suciu, and Lucian Mihai Itu. “Framework for Privacy-Preserving Wearable Health Data Analysis: Proof-of-Concept Study for Atrial Fibrillation Detection.” Applied Sciences 11, no. 19 (2021): 9049.

Wang, Jeen-Shing, et al. “ECG arrhythmia classification using a probabilistic neural network with a feature reduction method.” Neurocomputing 116 (2013): 38-45.

Wang, Jianqing, et al. “Wearable ECG Based on Impulse-Radio-Type Human Body Communication.” IEEE Trans. Biomed. Engineering 63.9 (2016): 1887-1894.

Wang, Robert, et al. “Accuracy of wrist-worn heart rate monitors.” Jama cardiology 2.1 (2017): 104-106.

Wang, Yishan, et al. “A wearable wireless ECG monitoring system with dynamic transmission power control for long-term homecare.” Journal of medical systems 39.3 (2015): 35.

Wannenburg, Johan, Reza Malekian, and Gerhard P. Hancke. “Wireless capacitive-based ECG sensing for feature extraction and mobile health monitoring.” IEEE Sensors Journal 18, no. 14 (2018): 6023-6032.

Welch Allyn Inc., TAGecg brochure, October 2018 (TAGecg).

Wong, A. C. W., D. McDonagh, O. Omeni, C. Nunn, M. Hernandez-Silveira, and A. J. Burdett. “Sensium: An ultra-low-power wireless body sensor network platform: Design & application challenges.” In 2009 Annual International Conference of the IEEE Engineering in Medicine and Biology Society, pp. 6576-6579. IEEE, 2009.

Wu, C-C., W-C. Kuo, H-J. Wang, Y-C. Huang, Y-H. Chen, Y-Y. Chou, S-A. Yu, and S-S. Lu. “A pliable and batteryless real-time ECG monitoring system-in-a-patch.” In VLSI Design, Automation and Test (VLSI-DAT), pp. 1-4. IEEE, 2015.

Xiong, Zhaohan, Martin K. Stiles, Anne M. Gillis, and Jichao Zhao. “Enhancing the detection of atrial fibrillation from wearable sensors with neural style transfer and convolutional recurrent networks.” Computers in Biology and Medicine 146 (2022): 105551.

Xu, Zhihong, Zhen Fang, Lidong Du, Zhan Zhao, Xianxiang Chen, Diliang Chen, Fangmin Sun, Yangming Qian, Huaiyong Li, and Lili Tian. “A Wearable Multi-parameter Physiological System.” In Ubiquitous Information Technologies and Applications, pp. 643-648. Springer, Berlin, Heidelberg, 2014.

Yan, Long, and Hoi-Jun Yoo. “A low-power portable ECG touch sensor with two dry metal contact electrodes.” Journal of semiconductor technology and science 10, no. 4 (2010): 300-308.

Yan, Long, Joonsung Bae, Seulki Lee, Binhee Kim, Taehwan Roh, Kiseok Song, and Hoi-Jun Yoo. “A 3.9 mW 25-electrode reconfigured thoracic impedance/ECG SoC with body-channel transponder.” In 2010 IEEE International Solid-State Circuits Conference-(ISSCC), pp. 490-491. IEEE, 2010.

Yan, Long, Joonsung Bac, Seulki Lec, Tachwan Roh, Kiscok Song, and Hoi-Jun Yoo. “A 3.9 mW 25-electrode reconfigured sensor for wearable cardiac monitoring system.” IEEE Journal of Solid-State Circuits 46, no. 1 (2010): 353-364.

Yang, Geng, et al. “A novel wearable ECG monitoring system based on active-cable and intelligent electrodes.” e-health Networking, Applications and Services, 2008. HealthCom 2008. 10th International Conference on. IEEE, 2008.

Yang, Zhe, et al. “An IoT-cloud based wearable ECG monitoring system for smart healthcare.” Journal of medical systems 40.12 (2016): 286.

Yao, Qihang, Ruxin Wang, Xiaomao Fan, Jikui Liu, and Ye Li. “Multi-class arrhythmia detection from 12-lead varied-length ECG using attention-based time-incremental convolutional neural network.” Information Fusion 53 (2020): 174-182.

Yao, Qihang, Xiaomao Fan, Yunpeng Cai, Ruxin Wang, Liyan Yin, and Ye Li. “Time-incremental convolutional neural network for arrhythmia detection in varied-length electrocardiogram.” In 2018 IEEE 16th Intl Conf on Dependable, Autonomic and Secure Computing, 16th Intl Conf on Pervasive Intelligence and Computing, 4th Intl Conf on Big Data Intelligence and Computing and Cyber Science and Technology Congress (DASC/PiCom/DataCom/CyberSciTech), pp. 754-761. IEEE, 2018.

Yeh, Yun-Chi, Che Wun Chiou, and Hong-Jhih Lin. “Analyzing ECG for cardiac arrhythmia using cluster analysis.” Expert Systems with Applications 39.1 (2012): 1000-1010.

Yildirim, Özal, Paweł Pławiak, Ru-San Tan, and U. Rajendra Acharya. “Arrhythmia detection using deep convolutional neural network with long duration ECG signals.” Computers in biology and medicine 102 (2018): 411-420.

Yoo, Jerald, and Hoi-Jun Yoo. “Emerging low energy wearable body sensor networks using patch sensors for continuous healthcare applications.” In 2010 Annual International Conference of the IEEE Engineering in Medicine and Biology, pp. 6381-6384. IEEE, 2010.

Yoo, Jerald, Long Yan, Seulki Lec, Hyejung Kim, and Hoi-Jun Yoo. “A wearable ECG acquisition system with compact planar-fashionable circuit board-based shirt.” IEEE Transactions on Information Technology in Biomedicine 13, no. 6 (2009): 897-902.

Yoo, Jerald, Long Yan, Seulki Lee, Yongsang Kim, and Hoi-Jun Yoo. “A 5.2 mW Self-Configured Wearable Body Sensor Network Controller and a 12ρW Wirelessly Powered Sensor for a Continuous Health Monitoring System.” IEEE journal of solid-state circuits 45, no. 1 (2009): 178-188.

Yoshimoto, Masahiko, and Shintaro Izumi. “Recent progress of biomedical processor SoC for wearable healthcare application: A review.” IEICE Transactions on Electronics 102, no. 4 (2019): 245-259.

Zadeh, Ataollah Ebrahim, Ali Khazace, and Vahid Ranace. “Classification of the electrocardiogram signals using supervised classifiers and efficient features.” computer methods and programs in biomedicine 99.2 (2010): 179-194.

Zhao, Luming, Hu Li, Jianping Meng, and Zhou Li. “The recent advances in self-powered medical information sensors.” InfoMat 2, no. 1 (2020): 212-234.

Zihlmann, M., D. Perekrestenko, M. Tschannen, Convolutional recurrent neural networks for electrocardiogram classification, arXiv preprint arXiv: 1710.06122 (2017).

See U.S. and foreign Pat. Nos. and Pat. Nos. 5,582,574; 5,658,277; 5,916,157; 6,327,795; 6,615,074; 6,842,999; 7,010,352; 7,036,245; 7,168,186; 7,337,559; 7,524,490; 7,805,849; 7,824,436; 7,837,722; 7,848,799; 7,877,900; 7,921,580; 7,950,971; 7539533; 7558622; 7733224; 8,075,605; 8,241,229; 8,290,577; 8,301,232; 8,313,520; 8,428,683; 8,430,805; 8,435,166; 8,441,356; 8,480,723; 8,494,507; 8,509,882; 8,571,622; 8,611,980; 8,630,633; 8,636,748; 8,669,864; 8,688,189; 8,700,137; 8,721,699; 8,738,112; 8,761,858; 8,805,475; 8,948,854; 8103333; 8323188; 8461988; 8525673; 8525687; 8531291; 8680991; 8684900; 8747336; 9,002,477; 9,005,102; 9,014,778; 9,022,949; 9,026,202; 9,040,101; 9,089,254; 9,173,670; 9,202,360; 9,220,430; 9,247,911; 9,254,092; 9,254,095; 9,307,921; 9,339,641; 9,351,654; 9,375,179; 9,387,338; 9,403,000; 9,420,956; 9,451,975; 9,463,169; 9,524,253; 9,572,499; 9,579,062; 9,610,459; 9,630,004; 9,649,042; 9,675,512; 9,681,814; 9,717,435; 9,737,225; 9,757,584; 9,775,741; 9,782,097; 9,804,635; 9,814,423; 9,833,158; 9,839,363; 9,867,990; 9,888,337; 9,894,471; 9,907,478; 9,911,290; 9028405; 9204796; 9351640; 9775520; 9901252; 9907473; 10,038,992; 10,049,182; 10,106,776; 10,111,643; 10,124,172; 10,140,820; 10,142,822; 10,159,415; 10,159,421; 10,165,355; 10,178,974; 10,187,773; 10,219,714; 10,285,608; 10,285,617; 10,355,730; 10,405,767; 10,413,733; 10,423,193; 10,441,602; 10,478,084; 10,478,623; 10,485,980; 10,510,219; 10,531,813; 10,537,250; 10,537,403; 10,548,500; 10,561,842; 10,573,134; 10,582,358; 10,586,623; 10307060; 10362940; 10517479; 10702179; 10716469; 10952794; 11033750; 11075009; 11083371; 11195616; 11246524; 11253186; 11260238; 11275595; 11278727; 11334826; 11337615; 11357439; 11375941; 11382555; 11389171; 11426089; 11443852; 11478184; 11490809; 11497432; 11504051; 20020023374; 20020157280; 20030032993; 20030199778; 20030212319; 20040006891; 20040123493; 20040134097; 20040138584; 20050283998; 20060041241; 20060099194; 20060156580; 20060224072; 20060264767; 20060276552; 20070009542; 20070123813; 20070144037; 20070149887; 20070190651; 20070273504; 20070276270; 20080001735; 20080004613; 20080234594; 20080249188; 20080263895; 20080281180; 20080306325; 20090037611; 20090130623; 20090210956; 20090227829; 20090227831; 20090227876; 20090234179; 20090234325; 20090318793; 20090318796; 20100005685; 20100036231; 20100056871; 20100115791; 20100160712; 20100168501; 20100204538; 20100211140; 20100222629; 20100298687; 20110021863; 20110028938; 20110040237; 20110046507; 20110046626; 20110115624; 20110160601; 20110181422; 20110265345; 20120053432; 20120083764; 20120092156; 20120092157; 20120095352; 20120109258; 20120165644; 20120302821; 20130009783; 20130053674; 20130072746; 20130096448; 20130150698; 20130172691; 20130184599; 20130211291; 20130231546; 20130237864; 20140046188; 20140046423; 20140068116; 20140077946; 20140094808; 20140104059; 20140143064; 20140148714; 20140148870; 20140163304; 20140163425; 20140213879; 20140276262; 20140303647; 20140328806; 20140343641; 20140358193; 20150068069; 20150073230; 20150094557; 20150105640; 20150141791; 20150141792; 20150202454; 20150234986; 20150335288; 20150351690; 20150351799; 20150359964; 20160029906; 20160067515; 20160067517; 20160074671; 20160140834; 20160183829; 20160193466; 20160206876; 20160246940; 20160249817; 20160262691; 20160287122; 20160287166; 20160302725; 20160331974; 20160359150; 20160360965; 20170034618; 20170055900; 20170135595; 20170215752; 20170215754; 20170225005; 20170231520; 20170265838; 20170266443; 20170333712; 20170340233; 20170344736; 20170347886; 20170354831; 20180050216; 20180055373; 20180085593; 20180146875; 20180168459; 20180184907; 20180199842; 20180233234; 20180247029; 20180279879; 20180316781; 20180317797; 20180368495; 20190008396; 20190038133; 20190040360; 20190076039; 20190147721; 20190151604; 20190167095; 20190182357; 20190213862; 20190214153; 20190223749; 20190239724; 20190246966; 20190247050; 20190254523; 20190259268; 20190261153; 20190261912; 20190290137; 20190307328; 20190320974; 20190336038; 20190341954; 20190350457; 20190363746; 20190366045; 20190387989; 20200000355; 20200000441; 20200069953; 20200077892; 20200107775; 20200108260; 20200118400; 20200118401; 20200118456; 20200121249; 20200126370; 20200139120; 20200160670; 20200161001; 20200205739; 20200215246; 20200273566; 20200281479; 20200281480; 20200305708; 20200323459; 20200352466; 20200372528; 20200405393; 20210022621; 20210093215; 20210100457; 20210121133; 20210145445; 20210146146; 20210186329; 20210212582; 20210244279; 20210244339; 20210252277; 20210259610; 20210275043; 20210313066; 20210315521; 20210330263; 20210338138; 20210343416; 20210345935; 20210345949; 20210350931; 20210353203; 20210361218; 20210369173; 20210369178; 20210379390; 20210402164; 20220000410; 20220001191; 20220008126; 20220023626; 20220039730; 20220061733; 20220068154; 20220068479; 20220068483; 20220079454; 20220088401; 20220105289; 20220107815; 20220107880; 20220117556; 20220133212; 20220143416; 20220160285; 20220165380; 20220167905; 20220183607; 20220225919; 20220225937; 20220249021; 20220296906; 20220296909; 20220304612; 20220304626; 20220313989; 20220323772; 20220330874; 20220330875; 20220338939; 20220355122; 20220355123; 20220361755; 20220382226; 20220386961; 20220386963; 20220387819; AU-2019372125; AU-2019379084; AU-2019385473; AU-2020269178; AU-2020367753; AU-2020385399; AU-2021206121; AU-2021218704; BR-1120170175112; CA-3070346; CA-3120522; CA-3124751; CA-3127747; CA-3145977; CA-3157536; CA-3159332; CA-3163990; CA-3165746; CA-3167546; CA-3171482; CA-3173752; D852,965; D854,167; EP-3256204; EP-3655055; EP-3713638; EP-38607102; EP-3873594; EP-3880105; EP-3884493; EP-3902459; EP-3906841; EP-3906843; EP-3906854; EP-39287022; EP-3933852; EP-3937181; EP-3937182; EP-3951788; EP-3960083; EP-3960085; EP-3965115; EP-3965643; EP-40215662; EP-4041070; EP-4061205; EP-4082027; EP-4087469; IN-201717029899; IN-202021015820; IN-202027001750; IN-202211029888; JP-2022040059; JP-2022042510; WO-2015092747; WO-2016128985; WO-2017027232; WO-2019016802; WO-2019104174; WO-2020072989; WO-2020092653; WO-2020101864; WO-2020106804; WO-2020136570; WO-2020172580; WO-2020226887; WO-2020243463; WO-2021007518; WO-2021038471; WO-2021076501; WO-2021094860; WO-2021102467; WO-2021127566; WO-2021130709; WO-2021140421; WO-2021163331; WO-2021167855; WO-2021174366; WO-2021181381; WO-2021252183; WO-2022020612; WO-2022079623; WO-2022082004; WO-2022130152; WO-2022140766; WO-2022153321; WO-2022159948; WO-2022191970; WO-2022194817; WO-2022241201; and WO-2022251145.

What is claimed is:

1. An electrocardiogram sensor, comprising:
an input port configured to receive an electrocardiogramal;
at least one automated processor, configured to:
process a representation of the electrocardiographic signal to determine an electrocardiographic waveform for a heartbeat cycle; and
encode a set of quantitative parameters from the electrocardiographic waveform, dependent on a plurality of relationships within the electrocardiographic waveform comprising both amplitude-dependent features and time-dependent features of the electrocardiographic waveform; and
a communication device, under control of the at least one automated processor, configured to communicate the encoded set of quantitative parameters through a communication channel.

2. The electrocardiogram sensor according to claim 1, wherein the set of quantitative parameters consists essentially of relative amplitude parameters, time duration parameters, and time difference parameters.

3. The electrocardiogram sensor according to claim 1, further comprising a memory configured to store a plurality of encoded sets of quantitative parameters, wherein the at least one automated processor is further configured to store the encoded set of quantitative parameters in the memory, and determine a type of an arrythmia from the encoded set of quantitative parameters for at least one heartbeat cycle, substantially without requiring the use of a stream of digitized values comprising the complete electrocardiographic signal.

4. The electrocardiogram sensor according to claim 1, further comprising a receiver device, configured to receive the encoded set of quantitative parameters through the communication channel, and distinguish a type of an arrythmia represented in the electrocardiographic signal with respect to a plurality of arrythmia types, selected from the group consisting of at least two of Left Bundle Branch Block, Right Bundle Branch Block, Premature Ventricular Contraction, Atrial Premature Beat, and Paced Beat.

5. The electrocardiogram sensor according to claim 1, further comprising:
an amplifier configured to amplify the electrocardiogramal; and
a digitizer configured to create a digital representation of the electrocardiographic signal,
wherein the at least one automated processor is configured to:
receive the digital representation as the representation of the electrocardiogramal,
determine a heart rate represented in the electrocardiographic waveform for the heartbeat cycle.

6. The electrocardiogram sensor according to claim 1, wherein the at least one automated processor is further configured to process the representation of the electrocardiographic signal to determine standardized electrocardiographic features, wherein the plurality of relationships within the electrocardiographic waveform comprising both amplitude-dependent features and time-dependent features of the electrocardiographic waveform are referenced to the determined standardized electrocardiographic features.

7. The electrocardiogram sensor according to claim 1, wherein the at least one automated processor is further configured to:
determine a respective power consumption mode dependent on analysis of the representation of the electrocardiographic signal; and
communicate different types of information packets through the communication channel containing respectively different information at different times selectively dependent on the determined respective power consumption mode.

8. The electrocardiogram sensor according to claim 1, wherein the at least one automated processor is further configured to:
predict an ability of a communication receiver to receive and process the encoded set of quantitative parameters through the communication channel, and
generate different types of information packets containing different information within the encoded set of quantitative parameters selectively dependent on the predicted ability of a communication receiver to receive and process the encoded set of quantitative parameters.

9. The electrocardiogram sensor according to claim 1, wherein the at least one automated processor is further configured to operate the electrocardiogram sensor in a plurality of different power consumption modes comprising respectively different duty cycles of operation of the communication device.

10. The electrocardiogram sensor according to claim 1, wherein the at least one automated processor is further configured to:
receive a stream of digitized electrocardiographic data;
maintain a data buffer storing the digitized electrocardiographic data;
periodically produce the encoded set of quantitative parameters, for inclusion in a data packet for communication through the communication channel; and
overwrite portions of the data buffer with subsequent digitized electrocardiographic data.

11. The electrocardiogram sensor according to claim 1, having a form factor comprising an adhesive flexible patch comprising electrocardiographic electrodes, the at least one automated processor, the communication device comprising a Bluetooth or Bluetooth low energy wireless transceiver, and a battery configured to power the electrocardiogram sensor.

12. The electrocardiogram sensor according to claim 1, wherein the at least one automated processor is further configured identify an R peak, QRS complex, P wave, and T wave within the electrocardiographic signal, and to encode the set of quantitative parameters based on at least the identified R peak, QRS complex, P wave, and T wave.

13. The electrocardiogram sensor according to claim 1, wherein the at least one automated processor is configured to encode the set of quantitative parameters consisting essentially of a plurality of relative amplitude features representing differences in amplitude of a digitized electrocardiographic waveform and a plurality of temporal features representing latency or relative latency of features of the electrocardiographic waveform within a period of the heartbeat cycle.

14. A method of operating an electrocardiogram sensor, comprising:
receiving an electrocardiogramal;
processing a representation of the electrocardiographic signal to determine an electrocardiographic waveform for a heartbeat cycle;
automatically encoding a set of quantitative parameters from the electrocardiographic waveform, dependent on time and amplitude features within a graphical representation of the respective electrocardiographic waveform; and
automatically controlling a communication of the encoded set of quantitative parameters through a wireless communication device.

15. The method according to claim 14, further comprising determining an existence and type of a cardiac arrythmia represented in the representation of the electrocardiographic signal dependent on the set of quantitative parameters.

16. The method according to claim 14, wherein the set of quantitative parameters consists essentially of relative amplitude parameters, time duration parameters, and time difference parameters.

17. The method according to claim 14, further comprising:
determining a respective power consumption mode dependent on at least one of:
an analysis of the representation of the electrocardiographic signal; and
a predicted ability of a communication receiver to receive and process the encoded set of quantitative parameters through the communication channel; and
communicating at least one of different types of information packets containing respectively different information and different rates of information packets, through the communication channel at different times selectively dependent on the determined respective power consumption mode.

18. The method according to claim 14, further comprising:
storing digitized electrocardiographic data in a buffer memory;
storing the encoded set of quantitative parameters for at least one heartbeat in the buffer memory;
periodically communicating the encoded set of quantitative parameters for at least one heartbeat through the wireless communication device;
communicating the stored digitized electrocardiographic data from the buffer memory through the wireless communication device based on a trigger; and
overwriting portions of the buffer memory storing the communicated encoded set of quantitative parameters while selectively preserving portions of the buffer memory storing the encoded set of quantitative parameters which have not yet been communicated.

19. The method according to claim 14, further comprising identifying at least an R peak, QRS complex, P wave, and T wave within the electrocardiographic signal, and encoding the set of quantitative parameters based on at least the identified R peak, QRS complex, P wave, and T wave.

20. A non-transitory computer-readable medium storing instructions for controlling an electrocardiogram sensor, comprising:
instructions for processing a representation of an electrocardiographic signal to determine features of an electrocardiographic waveform for a heartbeat cycle;
instructions for encoding a set of quantitative parameters from the electrocardiographic waveform, dependent on both feature amplitude relationships and feature time relationships of the electrocardiographic waveform for the heartbeat cycle; and
instructions for controlling a communication of the encoded set of quantitative parameters through a wireless communication device.
